(19) United States
(12) Patent Application Publication
(10) Pub. No.: US 2024/0309365 A1
(43) Pub. Date: Sep. 19, 2024

(54) MODULATING TRANSCRIPTIONAL CONDENSATES

(71) Applicant: Whitehead Institute for Biomedical Research, Cambridge, MA (US)

(72) Inventors: Richard A. Young, Boston, MA (US); Jonathan E. Henninger, Somerville, MA (US); Ozgur Oksuz, Worcester, MA (US)

(21) Appl. No.: 18/038,943

(22) PCT Filed: Nov. 24, 2021
(86) PCT No.: PCT/US2021/060735

§ 371 Date: May 25, 2023

Related U.S. Application Data
(60) Provisional application No. 63/118,586, filed on Nov. 25, 2020.

(51) Int. Cl. C12N 15/113; C12Q 1/6876

(52) U.S. Cl. CPC C12N 15/113; C12Q 1/6876; C12N 2310/11; C12Q 2600/158

(57) ABSTRACT

Disclosed herein are methods of modulating condensate dependent transcription of a gene by modulating condensate associated nucleic acids, including enhancer RNA and other regulatory RNA. Also disclosed are methods of screening for agents that modulate condensate dependent transcription of a gene by modulating condensate associated nucleic acids.

### RELATED APPLICATIONS

This application claims the benefit of U.S. Provisional Application No. 63/118,586, filed on Nov. 25, 2020. The entire teachings of the above application are incorporated herein by reference.

### GOVERNMENT SUPPORT

This invention was made with government support under Grant Nos. GM123511, CA155258 and 1F32CA254216-01 awarded by the National Institutes of Health, and Grant No. PHY-1743900 awarded by the National Science Foundation. The government has certain rights in the invention.

### BACKGROUND OF THE INVENTION

Mammalian transcription produces diverse RNA species from regulatory elements and genes and transcription of genes occurs in bursts of RNA synthesis. Transcription factors and coactivators recruit RNA polymerase II (Pol II) to enhancer and promoter elements, where short (20-400 bp) RNAs are bidirectionally transcribed before Pol II pauses. These RNA species are short-lived and are reported to have various regulatory roles, although there isn't yet a consensus on their functions. Pol II pause release leads to processive elongation, which occurs in periodic bursts (˜1-10 minutes in duration), where multiple molecules of Pol II can be released from promoters within a short timeframe and produce multiple molecules of mRNA (˜1-100 molecules per burst). How and whether the diverse RNA species produced during transcription-which differ in length, half-life, and number-impact or regulate transcription is currently unclear.

### SUMMARY OF THE INVENTION

Regulation of transcription is a fundamental cellular process that often goes awry in diseases. Transcription is regulated in part through the concentration and compartmentalization of large numbers of transcription factors, cofactors, and RNA polymerase II in liquid-like condensates. The ability to control the formation and dissolution of these condensates would thereby provide a method to control native and dysregulated transcription. The inventors demonstrate herein that the low levels of RNA present at transcription initiation promote transcriptional condensate formation, while the high levels of RNA produced during elongation promote condensate dissolution. RNA modulates transcriptional condensates principally via regulation of electrostatic interactions in transcriptional condensates. These results provide a simple, general, and powerful mechanism for both positive and negative regulation of transcription by polynucleotides and provide methods for discovery and drug development in the areas of oncogenic noncoding RNAs and RNA therapeutics.

Some aspects of the present disclosure are directed to a method of modulating condensate dependent transcription of a gene comprising modulating an amount, effective charge, structure, or behavior of nucleic acid incorporated in the condensate. In some embodiments, transcription of the gene is increased by less than 2-fold. In some embodiments, transcription of the gene is decreased by less than 50%. In some embodiments, condensate dependent transcription occurs in the presence of one or more species of regulatory RNA (regRNA) in the condensate. In some embodiments, the regRNA is tissue specific regRNA. In some embodiments, the regRNA is a variant associated with a disease or condition. In some embodiments, transcription is modulated by modulating the amount, effective charge, structure, or behavior of the one or more species of regRNA. In some embodiments, the amount of one or more species of regRNA is modulated by modulating transcription of the regRNA.

In some embodiments, transcription is modulated by contacting the one or more species of regRNA with an agent capable of specifically binding to the one or more species of regRNA. In some embodiments, the agent comprises a nucleic acid having a sequence complementary to a sequence of the one or more species of regRNA. In some embodiments, the nucleic acid is an antisense oligonucleotide or antisense RNA. In some embodiments, the one or more species of regRNA comprises enhancer RNA (eRNA). In some embodiments, the eRNA is a sequence variant associated with a disease or condition.

In some embodiments, the amount, effective charge, structure, or behavior of nucleic acid incorporated into the condensate is modulated by contact with an agent specifically binding to a nucleic acid associated with the condensate. In some embodiments, the agent is an oligonucleotide specifically binding a messenger RNA or portion thereof. In some embodiments, the condensate dependent transcription of a gene occurs in a cell. In some embodiments, the condensate dependent transcription of a gene occurs in vivo in a subject.

In some embodiments, the modulating of condensate dependent transcription of a gene treats, prevents or reduces the likelihood of a disease or condition in the subject. In some embodiments, the disease or condition is associated with a haploinsufficiency. In some embodiments, the disease or condition is associated with a gene duplication. In some embodiments, the disease or condition is associated with an eRNA variant.

Some aspects of the present disclosure are directed to a method of treating, preventing or reducing the likelihood of a disease or condition associated with aberrant condensate dependent transcription of a gene in a subject comprising administering to the subject an agent that modulates an amount, effective charge, structure, or behavior of nucleic acid in the condensate and thereby modulates transcription of the gene.

In some embodiments, the condensate dependent transcription occurs in the presence of one or more species of regulatory RNA (regRNA) in the condensate. In some embodiments, the agent comprises a nucleic acid having a sequence complementary to a sequence of the one or more species of regRNA. In some embodiments, the regRNA is an enhancer RNA (eRNA). In some embodiments, the disease or condition is associated with a haploinsufficiency. In some embodiments, the disease or condition is associated with a gene duplication. In some embodiments, the disease or condition is associated with an eRNA variant.

Some aspects of the present disclosure are directed to a method of treating, preventing or reducing the likelihood of a disease or condition in a subject comprising administering to the subject an agent that modulates an amount, effective charge, structure, or behavior of nucleic acid in a transcriptional condensate in a cell of the subject and thereby modulates transcription of a gene and treats, prevents, or reduces the likelihood of the disease or condition. In some embodiments, the transcription of the gene is increased. In some embodiments, a gene product of the gene is increased in the subject. In some embodiments, the agent comprises an oligonucleotide that specifically binds to a nucleic acid associated with the condensate. In some embodiments, transcription of the gene is decreased.

Some aspects of the present disclosure are directed to a method of identifying an agent that modulates a condensate, comprising providing a condensate comprising a regulatory RNA (regRNA), contacting the condensate with a test agent, and assessing whether the test agent dissolves or modulates the size of the condensate. In some embodiments, the regRNA is an enhancer RNA (eRNA). In some embodiments, the eRNA is an eRNA variant associated with a disease or condition. In some embodiments, the test agent specifically binds the regRNA. In some embodiments, the test agent comprises an antisense oligonucleotide or an antisense RNA. In some embodiments, the condensate comprises a detectable label. In some embodiments, the condensate is an in vitro condensate (e.g., a synthetic condensate or a condensate isolated from a cell) or is contained in a cell.

Some aspects of the present disclosure are directed to a method of identifying an agent that increases condensate formation, comprising providing a composition comprising a regulatory RNA (regRNA) and a condensate component under conditions wherein the concentration of the regRNA or condensate component does not form a condensate, contacting the composition with a test agent, and assessing whether contact with the test agent causes formation of a condensate. In some embodiments, the regRNA is an enhancer RNA (eRNA). In some embodiments, the eRNA is an eRNA variant associated with a disease or condition. In some embodiments, the test agent specifically binds the regRNA. In some embodiments, the test agent comprises an antisense oligonucleotide or an antisense RNA. In some embodiments, the regRNA or the condensate component comprises a detectable label. In some embodiments, the condensate is an in vitro condensate or is contained in a cell.

Some aspects of the present disclosure are directed to a method of identifying an agent that modulates condensate dependent transcription of a gene, comprising providing an in vitro transcription assay with condensate dependent expression of a reporter gene, contacting the in vitro transcription assay with a test agent, and assessing expression of the reporter gene, wherein condensate dependent expression requires incorporation of a regulatory RNA (regRNA) in the condensate. In some embodiments, the regRNA is an enhancer RNA (eRNA). In some embodiments, the eRNA is an eRNA variant associated with a disease or condition. In some embodiments, the test agent specifically binds the regRNA. In some embodiments, the test agent comprises an antisense oligonucleotide or an antisense RNA. In some embodiments, the condensate comprises a detectable label.

Some aspects of the present disclosure are directed to a method of identifying an agent that modulates condensate dependent transcription of a gene, comprising providing a cell with condensate dependent expression of a heterologous reporter gene, contacting the cell with a test agent, and assessing expression of the heterologous reporter gene, wherein condensate dependent expression requires incorporation of a regulatory RNA (regRNA) in the condensate. In some embodiments, the regRNA is an enhancer RNA (eRNA). In some embodiments, the eRNA is an eRNA variant associated with a disease or condition. In some embodiments, the test agent specifically binds the regRNA. In some embodiments, the test agent comprises an antisense oligonucleotide or an antisense RNA. In some embodiments, the condensate comprises a detectable label.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1H show that low levels of RNA enhance and high levels dissolve Mediator condensates. FIG. 1A—Diagram of reentrant phase transition in response to increasing concentration of RNA over constant protein concentration. The condensed fraction of protein peaks at the RNA concentration at which the charges between protein and RNA are balanced, while alteration of this charge balance in either direction decreases the condensed fraction. FIG. 1B—Experimental design for in vitro droplet formation assay. Whole Mediator complex is mixed with increasing concentrations of RNA under physiologically-relevant buffer conditions and droplets are imaged under confocal microscopy. FIG. 1C—Representative images of droplets formed by the unlabeled whole Mediator complex (200 nM) and Cy5-labeled Pou5f1 enhancer RNA at increasing concentrations (0-400 nM). Brightfield images of the Mediator complex were divided by a median-filtered image (px=15). FIG. 1D—Quantification of droplet sizes in (FIG. 1C). FIG. 1E—Quantification of partition ratios of Cy5-labeled RNA within the droplets in (FIG. 1C). Partition ratio is calculated as the mean intensity inside the droplets divided by the mean intensity outside the droplets. FIG. 1F—Representative images of droplets formed by the unlabeled whole Mediator complex (200 nM) and Cy5-labeled Trim28 enhancer RNA at increasing concentrations (0-400 nM). Brightfield images of the Mediator complex were divided by a median-filtered image (px=15). FIG. 1G—Quantification of droplet sizes in (FIG. 1F). FIG. 1H—Quantification of partition ratios of Cy5-labeled RNA within the droplets in (FIG. 1F).

FIGS. 2A-2D show RNA-mediated regulation of MED1-IDR condensates fits a charge balance model. FIG. 2A—Experimental design for in vitro droplet formation assay. Soluble MED1-IDR-GFP is mixed with increasing concentrations of RNA under physiologically relevant buffer conditions and droplets are imaged with confocal microscopy. FIG. 2B—Scheme of charge balance ratio between constant protein concentration and increasing RNA concentration. FIG. 2C—Representative images of droplets formed by increasing concentrations (0-400 nM) of the indicated RNAs mixed with 1 μM of MED1-IDR-GFP. FIG. 2D—Quantification of partition ratios of MED1-IDR-GFP within the droplets in (C) (left y-axis). Charge balance ratios between MED1-IDR-GFP and increasing concentrations of the indicated RNAs are shown in blue lines (right y-axis). Correlation between partition ratio and charge balance is determined by Pearson correlation (r).

FIGS. 3A-3I show RNA-mediated effects on condensates in reconstituted in vitro transcription assays. FIG. 3A—Cartoon representation of the reconstituted in vitro mammalian transcription assay with purified components (left) and the design of the assay (right). General transcription factors (TFIIA-B-D-E-F-H)), Mediator, GAL4 (Gal4 DNA binding domain fused to activation domain of VP16) and RNA Pol II are assembled on a template DNA containing a promoter with TATA-box and Gal4 binding sites. The transcription reaction is initiated by addition of NTPs. The transcription reaction is then subjected to indicated analyses. FIG. 3B—Brightfield images of droplets formed within the in vitro transcription reaction. Droplets are stained with DNA dye (Hoechst). Brightfield images were white tophat filtered and smoothed (STAR Methods). FIG. 3C—Brightfield images of droplets formed within the in vitro transcription reaction performed in the presence of indicated spermine concentrations. Template DNA is labeled with Cy3. Brightfield images were white tophat filtered and smoothed (STAR Methods). FIG. 3D—Quantification of droplet sizes in (FIG. 3C) (p=0.0011, Student's t-test). FIG. 3E—Quantification of partition ratio Cy3-labeled template DNA into the droplets in (FIG. 3C) (p<0.0001, Student's t-test). FIG. 3F—The effect of spermine on transcriptional output. Transcriptional output is measured by qRT-PCR. The relative levels of transcriptional output normalized to no spermine condition are indicated. The mean of 2 replicates are shown and error bars depict S.D. (p=0.0477, Student's t-test). FIG. 3G—The effect of increasing exogenous RNA levels on transcriptional condensates. Representative images of droplets in the in vitro transcription reaction in the presence of indicated amounts of exogenous RNA. Brightfield images were white tophat filtered and smoothed (STAR Methods). FIG. 3H—Quantification of droplet sizes in (FIG. 3G) (p=0.9309 0 vs. 10; p<0.001 for 0 vs. 50, 250, and 500, one-way ANOVA). FIG. 3I—The effect of increasing exogenous RNA levels on transcriptional output. Transcriptional output is measured by qRT-PCR. The relative levels of transcriptional output normalized to no RNA condition are indicated. The mean of 2 replicates are shown and error bars depict S.D. (p=0.0001 GTP only vs. 0; p=0.0111 0 vs. 10; p=0.0013 0 vs. 50; p=0.0008 0 vs. 250; p=0.008 0 vs. 500, one-way ANOVA).

FIGS. 4A-4H show a model for RNA-mediated non-equilibrium feedback control of transcriptional condensates. FIG. 4A—Schematic of coarse-grained free-energy (f, green-surface) which depends on the transcriptional protein (ϕₚ) and RNA (ϕᵣ) concentrations. This free-energy recapitulates in vitro observations of an equilibrium reentrant transition. FIG. 4B—Schematic of the non-equilibrium model coupling transcriptional activity with transcriptional condensate dynamics. In the model framework, we focus on a local micro-environment near a single transcriptional condensate (blue). RNA (magenta) is synthesized, degraded, and can diffuse. FIG. 4C—Equations underlying construction of the free-energy function (Equation 1) and dynamics of protein and RNA (Equation 2). The governing equations follow the outline in FIGS. 5A-B, and are described in the text and STAR Methods. FIG. 4D—Simulation predictions of transcriptional condensate lifetime (ordinate) with varying total protein concentrations (abcissa) (2D simulation grid). The dashed-line represents the lifetime of condensates that don't dissolve at steady state, and the ordinate is presented in units of simulation time. FIG. 4E and FIG. 4F—Simulation predictions of transcriptional condensate radius (FIG. 4E) and lifetime (FIG. 4F) at varying effective rates of RNA synthesis (abcissa) (2D simulation grid). The radius values are normalized to r=6.0 mesh units. he dashed line in F represents lifetime of stable condensates, which are presented in units of simulation time (STAR Methods). FIG. 4G—Variation of normalized condensate radius (y-axis, normalized to r=6.0 mesh units) with changing relative time-scales of reaction and diffusion (abcissa, t_{d}/tᵣ) (2D simulation grid). In these simulations, the total effective concentration of RNA produced is held constant (see text). The inset figure graphs the distribution of RNA concentrations at early simulation times (tₛₜₑₚ=100) for two different values of t_{d}/tᵣ (highlighted in the main panel with corresponding colors). FIG. 4H—Visualization of protein (blue) and RNA (magenta) concentration fields over simulation time for 3D simulations. The condensate is initialized (first panel) and then grows under low transcriptional activity (second panel). After a finite-time (tₛᵢₘ=1000), the effective rate of RNA synthesis (kₚ) is increased by 2.5-fold, which in turn, drives condensate shrinkage (third panel) and ultimately, dissolution (fourth panel) (STAR Methods).

FIGS. 5A-5I show inhibition of RNA elongation leads to enhanced condensate size and lifetime in cells. FIG. 5A—Scheme for preventing condensate dissolution upon transcriptional burst by treatment with small molecules that inhibit transcriptional elongation. FIG. 5B—Simulation predictions show variation of normalized condensate radius with total protein amount (abscissa) in absence (black, kₚ=0.1) and presence (red, kₚ=0.05) of RNA synthesis inhibition (2D simulation grid). The radius is normalized by the radius at kₚ=0.05, <P₀>=0.115. FIG. 5C—Experimental design to test the effect of transcriptional inhibition on the size of Mediator condensates. MED1-GFP mESCs are imaged by 3D super-resolution microscopy after treatment with small molecules. FIG. 5D—Max intensity projection images of single nuclei tagged with endogenous Med1-GFP in the presence of indicated transcriptional inhibitors or DMSO control. FIG. 5E—Quantification of the volume of Med1-GFP condensates in (D). p-value for DMSO vs. ActD<0.0001 and p-value for DMSO vs. DRB<0.0001, one-way ANOVA. FIG. 5F—Simulation predictions show variation of condensate lifetime with total protein amount (abscissa) in absence (black, kₚ=0.1) and presence (red, kₚ=0.05) of RNA synthesis inhibition (2D simulation grid). The lifetime is presented in units of simulation time. FIG. 5G—Experimental design to test the effect of DRB on the lifetime of Mediator condensates in Med19-tagged mESCs. Lifetimes are quantified by time-correlated PALM. FIG. 5H—Representative heatmap of Med19-Halo localizations in single nucleus upon addition of transcriptional inhibitor DRB, DRB wash or DMSO control. FIG. 5I—Cumulative distribution frequency plot of condensate lifetime in response to indicated treatments are shown (p<0.0001, one-way ANOVA).

FIGS. 6A-6G show increasing the levels of local RNA synthesis reduces condensate formation and transcription in cells. FIG. 6A—Scheme depicting the effect of increasing local RNA levels on transcriptional condensates and transcriptional output in the indicated reporter system (left). In this system, local RNA expression near a luciferase reporter gene can be induced by doxycycline. FIG. 6B—The experimental design to test the effect of increasing local RNA levels on condensate formation and on reporter gene expression. FIG. 6C—Live-cell imaging showing localization of Mediator condensates and MS2-tagged RNA expressed near the reporter gene at indicated dox stimulations. Med1-GFP mESCs have an integrated reporter system and 2×MCP-mCherry to visualize MS2-tagged RNA (2456 nt). Representative images are maximum projections that have been subtracted by a median filter and smoothed (STAR Methods). FIG. 6D—Average density of MED1 signal centered at RNA signal at indicated dox stimulations (p=0.066 10 ng/mL vs 100 ng/ml Dox; p=0.013 10 ng/mL vs 1000 ng/ml Dox, p=0.315 100 ng/mL vs 1000 ng/mL, 2-way Kolmongorov-Smirnoff test). FIG. 6E—Simulations predict the variation of condensate size with increasing effective rates of RNA synthesis (abscissa) (2D simulation grid). The condensate radius is normalized by value at rate=1 and RNA synthesis rates are normalized to kₚ=0.02 (STAR Methods). FIG. 6F—Quantification of RNA levels near luciferase reporter by qRT-PCR with increasing dox concentrations with various RNA species. Markers show the mean of at least 3 replicates and error bars depict the S.D. FIG. 6G—Quantification of luciferase luminescence with increasing dox concentrations to stimulate expression of various feedback RNA species. Markers show the mean of at least 3 replicates and error bars depict the S.D.

FIG. 7 shows a model for RNA-mediated feedback control of transcriptional condensates. Cartoon depicting a model whereby low levels of RNA present at transcription initiation promote condensate formation while high levels of RNA present during a transcriptional burst promote condensate dissolution.

FIGS. 8A-8F show transcription machinery and RNA at active genes in murine embryonic stem cells. FIG. 8A—A scheme of transcription states and the number of molecules and their corresponding effective charge in a typical transcriptional condensate during initiation and bursts of transcription (STAR Methods). FIGS. 8B-8D show enrichment of transcription machinery and RNA at Trim28 (FIG. 8B), Pou5f1 (FIG. 8C) and Nanog (FIG. 8D) super-enhancers in mESCs. Gene tracks of ChIP-seq and nascent RNA-seq data at the indicated super-enhancers are shown. The enhancer- and promoter-derived (sense) RNAs that are used in this study are annotated in the gene tracks. FIG. 8E—Nascent (left) or steady-state (right) levels of indicated RNAs at super and typical enhancers (eRNA=enhancer RNAs, uaRNA=upstream antisense promoter-associated RNAs, mRNA=messenger RNA). FIG. 8F-Quantification of the number of enhancer RNA and pre-mRNA molecules in cells. Calculations are based on two biological replicates (STAR Methods).

FIGS. 9A-9F characterize the effect of RNA on droplet formation and dissolution. FIG. 9A—Turbidity measurements of droplets formed with MED1-IDR-GFP and indicated RNAs. Correlation between partition ratio and charge balance is determined by Pearson correlation (r). FIG. 9B—Experimental design to test the effect of RNA on pre-formed MED1-IDR droplets (top). Representative images of MED1-IDR droplets and quantification of partition ratio of protein and RNA (bottom). Indicated concentrations of RNA were added after formation of droplets with 1 μM of MED1-IDR. FIG. 9C—Representative images of BRD4-IDR droplets at various RNA concentrations. FIG. 9D—Quantification of BRD4-IDR partition ratio from (FIG. 9C) and correlation with charge balance (blue lines). Correlation between partition ratio and charge balance is determined by Pearson correlation (r). FIG. 9E—Purified GFP (top) or OCT4-GFP (bottom) was incubated with an enhancer RNA from the Pou5f1 locus. Whereas this RNA could stimulate MED1-IDR-GFP condensate formation, it was unable to form droplets with GFP alone or OCT4-GFP. Images were adjusted to show signal and lack of droplet formation. FIG. 9F—FRAP analysis of droplets formed with MED1-IDR and RNA (top) or BRD4-IDR and RNA (bottom).

FIGS. 10A-10D show modulation of charge balance between MED1-IDR and RNA contributes to stimulation and dissolution of condensates. FIG. 10A—Experimental design for testing diverse sense and antisense RNAs of different lengths on formation of MED1-IDR-GFP droplets. FIG. 10B—Quantification of the partition ratios of MED1-IDR-GFP within the droplets when incubated with RNAs of different lengths and sequences. Correlation between partition ratio and charge balance is determined by Pearson correlation (r). FIG. 10C—Quantification of the partition ratios of MED1-IDR-GFP within the droplets when incubated with antisense versions of the RNAs in (B). Correlation between partition ratio and charge balance is determined by Pearson correlation (r). FIG. 10D—Representative images of MED1-IDR droplets (left), which are formed with or without RNA and are subjected to increasing concentration of monovalent salt (NaCl). Quantification of partition ratios of MED1-IDR-GFP within the droplets are indicated (right).

FIGS. 11A-11H show formation and dissolution of MED1-IDR droplets through electrostatic interactions. FIG. 11A—Representative images for MED1-IDR-GFP in a two-component phase diagram for MED1-IDR and Pou5f1 eRNA. FIG. 11B—Quantification of MED1-IDR partition ratio for (FIG. 11A). FIG. 11C—Representative images for RNA-Cy5 in a two-component phase diagram for MED1-IDR and Pou5f1 eRNA in (FIG. 11A). FIG. 11D—Quantification of RNA-Cy5 partition ratio and charge balance ratio for (FIG. 11C). FIG. 11E—Representative images of droplets formed with single-stranded DNA (top) or heparin (bottom). FIG. 11F—Quantification of MED1-IDR partition ratio for images in (FIG. 11E). Correlation between partition ratio and charge balance is determined by Pearson correlation (r). FIG. 11G—Representative images of droplets formed with MED1-IDR RHK>A and Pou5f1 enhancer RNA. FIG. 11H—Quantification of MED1-IDR partition ratio for images in (FIG. 11G).

FIGS. 12A-12D show charged interactions in reconstituted mammalian transcription and droplets. FIG. 12A—Representative images of transcription reactions with addition of 2 mM NTPs, 200 mM NaCl, and 500 nM Heparin. FIG. 12B—Quantification of droplet area of droplets in (FIG. 12A). FIG. 12C—Quantification of DNA-Cy3 partitioning from droplets in (FIG. 12A). FIG. 12D—qRT-PCR measurement of template-derived RNA synthesis in the reconstituted transcription reactions from (FIG. 12A).

FIGS. 13A-13E show computational model for non-equilibrium RNA feedback on transcriptional condensates. FIG. 13A—Regions where mixtures of protein and RNA phase separate spontaneously (red, left panel) are calculated from the Landau free-energy (FIG. 4C) by analyzing the Jacobian (spinodal analyses, STAR Methods). As expected from the re-entry transition, increasing RNA concentration (abscissa) at fixed protein levels can start from a region promoting phase separation, and beyond a threshold, drive re-entry into dilute phase. The right panel shows the initial direction of the instability (STAR Methods), which indicates the RNA is enriched in protein condensates (value>0, green shade), while at higher concentrations, RNA de-densifies the condensed phase (value<0). FIG. 13B—Similar analyses as in (FIG. 13A) are performed on a free-energy derived from Flory-Huggins model (STAR Methods). FIG. 13C—Variation of condensate radius (left panel, normalized to value of R at kₚ=0.02) and condensate lifetime (right panel) with effective rates of RNA synthesis (kₚ, abscissa) for simulations performed in 3D employing the Landau free-energy (FIG. 4C, STAR Methods). Low values of kₚ promote condensate stability whereas higher rates drive dissolution. The dashed line in the right-panel represents the conditions under which condensates are stable in the simulations and condensate lifetime is presented in units of simulation time (STAR Methods). FIG. 13D—Similar analyses as in (FIG. 13C) are performed on a free-energy derived from Flory-Huggins model on a 2D grid (STAR Methods). Values of the condensate radius are normalized to value of R at kₚ=0.08. FIG. 13E—Partition ratio, computed as maximum RNA concentration in condensate divided by dilute phase concentrations, are presented for simulations employing the Landau free-energy in 2 & 3-D as well as those employing the Flory-Huggins model in 2-D (left to right). When condensates are dissolved, the expected value of this ratio is 1 (as depicted by dashed gray lines). These calculations correspond to simulation data from FIGS. 4D-4E, 13C, and 13D, respectively (left to right).

FIGS. 14A-14J show the effect of local RNA synthesis on transcriptional condensates and transcription in cells. FIG. 14A—Immunofluorescence of MED1 and POL2-S2 with EU-labeled RNA (10 minute incubation) in WT mESCs. Representative images are a single z-plane that has been subtracted by a median-filtered image and smoothed (STAR Methods). FIG. 14B—Average signal analysis of EU-RNA, MED1 and POL2-S2. Average EU-RNA signal is centered at MED1 puncta (top) or at POL2-S2 puncta (bottom). FIG. 14C—Radial distribution function with correlation between EU and MED1 (top) or POLII-S2 (bottom) channels for average IF signal in (FIG. 14B). FIG. 14D—Radial distribution function of MED1-GFP signal for multiple dox concentrations from experiments in FIG. 6C. FIG. 14E—Quantification of luciferase luminescence with increasing dox concentrations to stimulate expression of short (185 nt) and long (1313 nt) feedback RNAs with indicated orientations of feedback RNA and luciferase gene. The convergent data was collected as part of FIG. 6G. Markers show the mean of at least 3 replicates and error bars depict the S.D. FIG. 14F—Quantification of luciferase luminescence with increasing dox concentration to stimulate expression of short (185 nt) and long (1313 nt) feedback RNAs with cis or trans integration of feedback RNA and luciferase reporter gene. The cis data was collected as part of FIG. 6G. Markers show the mean of at least 3 replicates and error bars depict the S.D. FIG. 14G—RT-qPCR of neighboring puromycin resistance marker gene with and without stimulation of long (1313 nt) feedback RNA expression with dox. FIG. 14H—RT-qPCR of luciferase mRNA gene with primers detecting truncated and full-length RNA with and without stimulation of long (1313 nt) feedback RNA expression with dox. FIG. 14I—Quantification of luciferase luminescence with increasing dox concentration to stimulate expression of short (185 nt) and long (1313 nt) feedback RNAs, before and after dox washout. FIG. 14J—The effect of antisense-oligo mediated degradation of feedback RNAs on luciferase luminescence. The expression of the feedback RNAs are measured by RT-qPCR (left bar graphs) and luciferase expression is measured by luminescence (right bar graphs). The luminescence values were first normalized to the no dox condition for that ASO, and then normalized to the dox condition of the negative control ASO.

### DETAILED DESCRIPTION OF THE INVENTION

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N. Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies-A Laboratory Manual, Cold Spring Harbor Laboratory Press, Cold Spring Harbor, 1988; Freshney, R.I., “Culture of Animal Cells, A Manual of Basic Technique”, 5th ed., John Wiley & Sons, Hoboken, N J, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, MD) and National Center for Biotechnology Information, National Library of Medicine (Bethesda, MD), as of May 1, 2010, ncbi.nlm.nih.gov/omim/and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

It is shown herein that RNA acts as a powerful mediator of transcription via the control of transcriptional condensate formation and dissolution. Transcription becomes dysregulated in many diseases, most notably during oncogenesis, and the results shown herein provide a framework for new entry points in therapeutics that target transcriptional processes, which have been notoriously difficult to target. Described herein is a previously unknown feature of transcription, showing that bursts of RNA transcription can lead to the dissolution of transcriptional condensates. Further, the results herein show that noncoding RNA, such as enhancer RNA, may play a role in transcription regulation through modulating transcriptional condensate formation, dissolution and stability.

The ability to deploy RNA in order to control local electrostatic interactions within transcriptional condensates permits tuning of transcriptional output. In addition, various oncogenic noncoding RNAs or therapeutic RNAs like antisense oligos are rapidly being developed and fit in the framework of RNA-mediated feedback control of transcription. These therapeutics provide opportunities for targeting transcriptional processes in disease.

Some aspects of the present disclosure are directed to a method of modulating condensate dependent transcription of a gene comprising modulating an amount, effective charge, structure, or behavior of nucleic acid incorporated in the condensate. The condensate may be a transcriptional condensate (in a cell or isolated from a cell) or a synthetic transcriptional condensate (sometimes referred to herein as a synthetic or artificial condensate).

Transcriptional condensates are phase-separated multi-molecular assemblies that occur at the sites of transcription and are high density cooperative assemblies of multiple components that can include transcription factors, co-factors, chromatin regulators, DNA, non-coding RNA, nascent RNA, and RNA polymerase II. In some instances, transcriptional condensates are formed by super-enhancer assemblies. Many diseases are caused by, or associated with, alteration in these nucleic acid and protein components, and therapeutic intervention may be afforded by altering transcriptional output of condensates. As used herein, a synthetic transcriptional condensate refers to a non-naturally occurring condensate comprising one or more transcriptional condensate components.

As used herein “modulating” (and verb forms thereof, such as “modulates”) means causing or facilitating a qualitative or quantitative change, alteration, or modification. Without limitation, such change may be an increase or decrease in a qualitative or quantitative aspect.

The terms “increased,” “increase” or “enhance” may be, for example, increase or enhancement by a statically significant amount. In some instances, for example, an element can be increased or enhanced by at least about 10% as compared to a reference level (e.g., a control), at least about 20%, at least about 30%, at least about 40%, at least about 50%, at least about 60%, at least about 70%, at least about 80%, at least about 90%, or at least about 100%, and these ranges will be understood to include any integer amount therein (e.g., 2%, 14%, 28%, etc.) which are not exhaustively listed for brevity. In other instances, an element can be increased or enhanced by at least about 2-fold, at least about 3-fold, at least about 4-fold, at least about 5-fold at least about 10-fold or more as compared to a reference level.

The terms “decrease,” “reduce,” “reduced,” “reduction,” and “inhibit” may be, for example, a decrease or reduction by a statistically significant amount relative to a reference (e.g., a control). In some instances an element can be, for example, decreased or reduced by at least 10% as compared to a reference level, by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the element as compared to a reference level. These ranges will be understood to include any integer amount therein (e.g., 6%, 18%, 26%, etc.) which are not exhaustively listed for brevity.

For example, modulating transcription of a gene includes increasing or decreasing the rate or frequency of gene transcription; modulating an amount of nucleic acid incorporated in the condensate includes increasing or decreasing the amount of the nucleic acid incorporated in the condensate; modulating an effective charge of nucleic acid incorporated in the condensate includes increasing or decreasing the effective charge of the nucleic acid; modulating a shape of nucleic acid incorporated into the condensate includes modifying the shape of the nucleic acid including minor modifications in shape as well as significant modifications in shape; and modulating behavior of nucleic acid incorporated into the condensate includes modifying the binding, localization, and/or stability of the nucleic acid.

In some embodiments, transcription of the gene or a level of gene product is increased by 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more as compared to a reference level (e.g., an untreated control cell or condensate). In some embodiments, transcription of the gene or a level of gene product is increased by less than 2-fold. In some embodiments, transcription of the gene or a level of gene product is increased by 1.1 to 1.5-fold. In some embodiments, transcription of the gene or a level of gene product is increased by 1.5 to 1.9-fold.

In some embodiments, transcription of the gene or a level of gene product is reduced by at least about 5%, 10%, 15%, 20%, 25%, 30%, 35%, 40%, 45%, 50%, 55%, 60%, 65%, 70%, 75%, 80%, 85%, 90%, 95%, 98%, 99%, 99.5%, 99.9%, or more as compared to a reference level (e.g., an untreated control cell or condensate). In some embodiments, transcription of the gene or a level of gene product is decreased by less than 50%. In some embodiments, transcription of the gene or a level of gene product is decreased by between about 10% and 50%. In some embodiments, transcription of the gene or a level of gene product is decreased by between about 5% and 25%.

In some embodiments, the rate of gene transcription is modulated. In some embodiments, the amount of time during which transcription occurs is modulated. For example, for burst transcription, the period of time during which transcription is occurring during one or more of the burst transcription events can be increased by, e.g., increasing the stability of the condensate by modulating nucleic acid incorporated into the condensate. In other embodiments, the period of time during which transcription is occurring during one or more of the burst transcription events can be decreased by, e.g., decreasing the stability of the condensate by modulating nucleic acid incorporated into the condensate. In some embodiments, the period of time between burst transcription events can be modulated by, e.g., inhibiting or enhancing condensate formation by modulating an amount, effective charge, structure, or behavior of nucleic acid incorporated in the condensate.

The gene for which condensate transcription is modulated is not limited. In some embodiments, the gene is an oncogene. Exemplary oncogenes include MYC, SRC, FOS, JUN, MYB, RAS, ABL, HOXI1, HOXI1 1L2, TAL1/SCL, LMO1, LMO2, EGFR, MYCN, MDM2, CDK4, GLI1, IGF2, activated EGFR, mutated genes, such as FLT3-ITD, mutated of TP53, PAX3, PAX7, BCR/ABL, HER2/NEU, FLT3R, FLT6-ITD, SRC, ABL, TAN1, PTC, B-RAF, PML-RAR-alpha, E2A-PRX1, and NPM-ALK, as well as fusion of members of the PAX and FKHR gene families. Other exemplary oncogenes are well known in the art. In some embodiments the oncogene is selected from the group consisting of c-MYC and IRF4. In some embodiments the gene encodes an oncogenic fusion protein, e.g., an MLL rearrangement, EWS-FLI, ETS fusion, BRD4-NUT, NUP98 fusion.

In some embodiments, the gene is associated with a hallmark of a disease such as cancer (e.g., breast cancer). In some embodiments, the gene is associated with a disease associated DNA sequence variation such as a SNP. In some embodiments, the disease is Alzheimer's disease, and the gene is BIN1. In some embodiments, the disease is type 1 diabetes, and the gene is associated with a primary Th cell. In some embodiments, the disease is systemic lupus erythematosus, and the gene plays a key role in B cell biology. In some embodiments, the gene is associated with a disease or condition associated with a mutation in a gene encoding a nuclear receptor. In some embodiments, the gene is associated with a hallmark characteristic of the cell. In some embodiments, the gene is aberrantly expressed or is associated with a DNA variation such as a SNP. “Aberrantly expressed” is used to indicate that the gene expression in one or more cells or synthetic condensates is detectably different from a control level that is typical of that found in normal cells (e.g., normal cells of the same cell type or, for cultured cells, cultured cells under comparable conditions) or condensates not subject to a test treatment or condition (e.g., for condensates isolated from cells, isolated condensates from normal cells of the same cell type or, for cultured cells, cultured cells under comparable conditions). In some embodiments, the gene is associated with aberrant signaling in a cell (e.g. aberrant signaling associated with the WNT, TGF-β or JAK/STAT pathways). In some embodiments, the gene exhibits aberrant mRNA initiation or elongation (e.g., aberrant splicing). As used herein, “aberrant mRNA initiation or elongation” is detectably or significantly different than mRNA initiation or elongation in a control cell or subject (e.g., higher than or lower than in (increased or decreased as compared to) a healthy cell or subject, or cell or subject without a disease or condition characterized by atypical mRNA initiation or elongation). In some embodiments, the gene is associated with a disease or disorder associated with aberrant gene silencing (e.g., increased or decreased gene silencing as compared to gene silencing in a healthy cell or healthy subject (e.g., control cell or subject)). In some embodiments, the disease or disorder associated with aberrant gene silencing is Rett syndrome, MeCP2 over-expression syndrome or MeCP2 under-expression or activity. MeCP2 refers to methyl CpG binding protein 2 (Human UniProt ID: P51608).

In some embodiments, the gene is found in a mammalian cell, e.g., human cell; fetal cell; embryonic stem cell or embryonic stem cell-like cell, e.g., cell from the umbilical vein, e.g., endothelial cell from the umbilical vein; muscle, e.g., myotube, fetal muscle; blood cell, e.g., cancerous blood cell, fetal blood cell, monocyte; B cell, e.g., Pro-B cell; brain, e.g., astrocyte cell, angular gyrus of the brain, anterior caudate of the brain, cingulate gyrus of the brain, hippocampus of the brain, inferior temporal lobe of the brain, middle frontal lobe of the brain, brain cancer cell; T cell, e.g., naïve T cell, memory T cell; CD4 positive cell; CD25 positive cell; CD45RA positive cell; CD45RO positive cell; IL-17 positive cell; a cell that is stimulated with PMA; Th cell; Th17 cell; CD255 positive cell; CD127 positive cell; CD8 positive cell; CD34 positive cell; duodenum, e.g., smooth muscle tissue of the duodenum; skeletal muscle tissue; myoblast; stomach, e.g., smooth muscle tissue of the stomach, e.g., gastric cell; CD3 positive cell; CD14 positive cell; CD19 positive cell; CD20 positive cell; CD34 positive cell; CD56 positive cell; prostate, e.g., prostate cancer; colon, e.g., colorectal cancer cell; crypt cell, e.g., colon crypt cell; intestine, e.g., large intestine; e.g., fetal intestine; bone, e.g., osteoblast; pancreas, e.g., pancreatic cancer; adipose tissue; adrenal gland; bladder; esophagus; heart, e.g., left ventricle, right ventricle, left atrium, right atrium, aorta; lung, e.g., lung cancer cell; skin, e.g., fibroblast cell; ovary; psoas muscle; sigmoid colon; small intestine; spleen; thymus, e.g., fetal thymus; breast, e.g., breast cancer; cervix, e.g., cervical cancer; mammary epithelium; liver, e.g., liver cancer; DND41 cell; GM12878 cell; H1 cell; H2171 cell; HCC1954 cell; HCT-116 cell; HeLa cell; HepG2 cell; HMEC cell; HSMM tube cell; HUVEC cell; IMR90 cell; Jurkat cell; K562 cell; LNCaP cell; MCF-7 cell; MM1S cell; NHLF cell; NHDF-Ad cell; RPMI-8402 cell; U87 cell; VACO 9M cell; VACO 400 cell; or VACO 503 cell.

In some embodiments, the gene is a disease-associated variation related to rheumatoid arthritis, multiple sclerosis, systemic scleroderma, primary biliary cirrhosis, Crohn's disease, Graves disease, vitiligo and atrial fibrillation. In some embodiments, the gene is associated with a developmental disorder. In some embodiments, the gene is associated with a neurological disorder or developmental neurological disorder.

In some embodiments, the gene is considered cell type specific. A cell type specific gene need not be expressed only in a single cell type but may be expressed in one or several, e.g., up to about 5, or about 10 different cell types out of the approximately 200 commonly recognized (e.g., in standard histology textbooks) and/or most abundant cell types in an adult vertebrate, e.g., mammal, e.g., human. In some embodiments, a cell type specific gene is one whose expression level can be used to distinguish a cell, e.g., a cell as disclosed herein, such as a cell of one of the following types from cells of the other cell types: adipocyte (e.g., white fat cell or brown fat cell), cardiac myocyte, chondrocyte, endothelial cell, exocrine gland cell, fibroblast, glial cell, hepatocyte, keratinocyte, macrophage, monocyte, melanocyte, neuron, neutrophil, osteoblast, osteoclast, pancreatic islet cell (e.g., a beta cell), skeletal myocyte, smooth muscle cell, B cell, plasma cell, T cell (e.g., regulatory, cytotoxic, helper), or dendritic cell. In some embodiments a cell type specific gene is lineage specific, e.g., it is specific to a particular lineage (e.g., hematopoietic, neural, muscle, etc.) In some embodiments, a cell-type specific gene is a gene that is more highly expressed in a given cell type than in most (e.g., at least 80%, at least 90%) or all other cell types. Thus specificity may relate to level of expression, e.g., a gene that is widely expressed at low levels but is highly expressed in certain cell types could be considered cell type specific to those cell types in which it is highly expressed. In some embodiments, a cell-type specific gene is a gene that is less expressed, or not expressed, in a given cell type than in most (e.g., at least 80%, at least 90%) or all other cell types. Thus specificity may relate to level of expression, e.g., a gene that is widely expressed but is much less expressed in certain cell types could be considered cell type specific to those cell types in which it is less, or not at all, expressed. It will be understood that expression can be normalized based on total mRNA expression (optionally including miRNA transcripts, long non-coding RNA transcripts, and/or other RNA transcripts) and/or based on expression of a housekeeping gene in a cell. In some embodiments, a gene is considered cell type specific for a particular cell type if it is expressed at levels at least 2, 5, or at least 10-fold greater or less than in that cell than it is, on average, in at least 25%, at least 50%, at least 75%, at least 90% or more of the cell types of an adult of that species, or in a representative set of cell types. One of skill in the art will be aware of databases containing expression data for various cell types, which may be used to select cell type specific genes. In some embodiments a cell type specific gene is a transcription factor. In some embodiments, a cell type specific gene is associated with embryonic, fetal, or post-natal development.

In some embodiments, the disease or condition is associated with aberrant expression or activity of one or more regulatory RNAs. For example, miRNA and lncRNA have been implicated in cancer, cardiovascular disease, neurodegenerative disease (Parkinson's disease, Alzheimer's disease, Huntington's disease, spinal muscular atrophy, frontotemporal lobar degeneration, amyotrophic lateral sclerosis). See, Lekka, E., & Hall, J. (2018) “Noncoding RNAs in disease,” FEBS letters, 592(17), 2884-2900. In some embodiments, the disease or condition is associated with haploinsufficiency, gene duplication, or an enhancer RNA variant.

In some embodiments, condensate dependent transcription occurs in the presence of one or more species of regulatory RNA (regRNA) in the condensate. As used herein, “regulatory RNA” are functional RNA molecules that are not translated into proteins. Types of regulatory RNA include microRNAs (miRNA), which are about 22 nucleotides long RNA molecules that in animals regulate gene expression post-transcriptionally in a sequence-specific manner, by facilitating messenger RNA (mRNA) degradation or by controlling translation. Other small regRNAs include: PIWI-interacting RNA (piRNA), about 28 nucleotides long RNA molecules involved in transposon repression and DNA methylation; small nucleolar RNA (snoRNA), about 60-300 nucleotides long, components of small nucleolar ribonucleoproteins, which modulate biogenesis and activity of ribosomes by post-transcriptional modifications of ribosomal RNA (rRNA); and small nuclear RNA (snRNA), about 150 nucleotides long RNA molecules that facilitate mRNA splicing and regulate transcription factors. In some embodiments, regRNA include promoter-associated RNA, including but not limited to, a promoter upstream transcript (PROMPT), a promoter-associated long RNA (PALR), and a promoter-associated small RNA (PASR). In further embodiments, regRNAs may include but are not limited to transcription start sites (TSS)-associated RNAs (TSSa-RNAs), transcription initiation RNAs (tiRNAs), and terminator-associated small RNAs (TASRs). In specific examples, the regRNA is an enhancer RNA (eRNA). As used herein, enhancer RNA (eRNA) refers to a class of relatively short non-coding RNA molecules (50-2000 nucleotides) transcribed from the DNA sequence of an enhancer region, such as a super-enhancer. In some embodiments, the eRNA is identified in the HACER database available on the world-wide web at bioinfo.vanderbilt.edu/AE/HACER/. In some embodiments, the eRNA is Trim28 eRNA, Pou5f1 eRNA, Oct4 eRNA, or Nanog eRNA. In some embodiments, the eRNA can be found in a database of transcribed human enhancers available on the world-wide web at fantom.gsc.riken.jp/5/. In some embodiments, the eRNA can be found in Andersson, et al., Nature 507, 455-461.

In some embodiments, the regRNA is tissue, organ or cell specific regRNA. The tissue, organ, or cell type is not limited and may be any tissue, organ, or cell type mentioned herein with regard to genes specific to cells, tissues or organs. As is apparent to a person of skill in the art, the capability to modulate a widely expressed gene only in a specific cell, tissue or organ type by modulating regRNA (e.g., eRNA) uniquely expressed in the specific cell, tissue or organ type has wide applicability for therapy and research.

In some embodiments, the regRNA is a variant associated with a disease or condition. Recent genome-wide association studies (GWAS) have found >88% of disease-risk variants lie in non-coding regions (Hindorff et al., “Potential etiologic and functional implications of genome-wide association loci for human diseases and traits,” Proc. Natl. Acad. Sci. U.S.A. 2009; 106:9362-9367), especially enriched in enhancers (Corradin et al., “Enhancer variants: evaluating functions in common disease,” Genome Med. 2014; 6:85). In some embodiments, the regRNA is an eRNA associated with an enhancer region comprising a SNP correlated with a disease or condition in a GWAS. In some embodiments, the enhancer region is part of or associated with the Trim28, Pou5f1, Oct4, or Nanog gene.

In some embodiments, the regRNA (e.g., eRNA) is associated with or part of a gene described above (e.g., a gene associated with a disease or condition, a cell specific gene, a tissue or organ specific gene.

In some embodiments, transcription is modulated by modulating the amount, effective charge, structure, or behavior of the one or more species of regRNA (e.g., eRNA). In some embodiments, the amount of one or more species of regRNA is modulated by modulating transcription of the regRNA. In some embodiments, transcription of the eRNA is increased. In some embodiments, transcription of the eRNA is decreased or prevented. In some embodiments, the transcription of the one or more species of regRNA (e.g., eRNA) is modulated by introducing a mutation in a DNA region (e.g., promoter) controlling transcription of the eRNA. In some embodiments, the effective charge, structure, or behavior of the one or more species of regRNA (e.g., eRNA) is modulated by introducing a mutation in the DNA transcribing the eRNA. In some embodiments, the introduced mutation changes the effective charge of the eRNA in the condensate. In some embodiments, the introduced mutation changes the shape of the eRNA in the condensate. In some embodiments, the introduced mutation changes the affinity of the eRNA for associating with the condensate or one or more components forming or associated with the condensate. In some embodiments, the mutation is introduced by contact with an agent as described herein (e.g., a targeting endonuclease).

As used herein, the phrases “a component associated with a condensate” or the like and the phrase “a condensate component” or the like refer to a peptide, protein, nucleic acid, signaling molecule, lipid, or the like that is part of a condensate or has the capability of being part of a condensate (e.g., transcriptional or synthetic condensate). In some embodiments, the component is within the condensate. In some embodiments, the component is on the surface of the condensate. In some embodiments, the component is necessary for condensate formation or stability. In some embodiments, the component is not necessary for condensate formation or stability. In some embodiments, the component is a protein or peptide and comprises one or more intrinsically ordered domains (e.g., an IDR of an activation domain of a transcription factor, an IDR that interacts with an IDR of an activation domain of a transcription factor, an IDR of a signaling factor, an IDR of a polymerase,). In some embodiments, the component is a non-structural member of a condensate (e.g., not necessary for condensate integrity) and is sometimes referred to as a client component. In some embodiments, a condensate comprises, consists of, or consists essentially of 1, 2, 3, 4, 5, 6, 7, 8, 9, 10 or more components. In some embodiments, the component is a fragment of a protein or nucleic acid. In some embodiments, the transcriptional condensate components comprise transcription factors, co-factors, chromatin regulators, DNA, non-coding RNA, nascent RNA, RNA polymerase II, kinases, proteasomes, topoisomerase, and/or enhancers. In some embodiments, the transcription factor is, e.g., OCT4, p53, MYC, GCN4, NANOG, MyoD, KLF4, a SOX family transcription factor, a GATA family transcription factor, a nuclear receptor, or a fusion oncogenic transcription factor. In some embodiments, the component is mediator, a mediator component, MED1, BRD4, POLII (i.e., POL2). In some embodiments, the transcriptional condensate component is a fragment of a transcriptional condensate component described herein comprising an IDR. Regions of intrinsic disorder, also termed intrinsic (or intrinsically) disordered regions (IDR) or intrinsic (or intrinsically) disordered domains can be found in many protein condensate components. Each of these terms is used interchangeably throughout the disclosure. IDR lack stable secondary and tertiary structure. In some embodiments, an IDR may be identified by the methods disclosed in Ali, M., & Ivarsson, Y. (2018). High-throughput discovery of functional disordered regions. Molecular Systems Biology, 14(5), e8377. IDRs are known in the art and any suitable method may be used to identify an IDR.

In some embodiments, transcription is modulated by contacting the one or more species of regRNA with an agent. The agent is not limited and may be any suitable agent.

“Agent” is used herein to refer to any substance, compound (e.g., molecule), supramolecular complex, material, or combination or mixture thereof. In some aspects, an agent can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, peptide mimetics, etc. In general, agents may be obtained using any suitable method known in the art. The ordinary skilled artisan will select an appropriate method based, e.g., on the nature of the agent. An agent may be at least partly purified or be substantially pure. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. Unless otherwise specified, the term “analog” as used herein refers to a structural analog. A structural analog of an agent may have substantially similar physical, chemical, biological, and/or pharmacological propert(ies) as the agent or may differ in at least one physical, chemical, biological, or pharmacological property. In some embodiments at least one such property differs in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog.

In some embodiments, the agent is a nucleic acid. The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, ST (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long. In some embodiments, the nucleic acid agent codes for a gene product of the testis specific gene or X-linked homolog thereof.

“Nucleic acid construct” refers to a nucleic acid that is generated by man and is not identical to nucleic acids that occur in nature, i.e., it differs in sequence from naturally occurring nucleic acid molecules and/or comprises a modification that distinguishes it from nucleic acids found in nature. A nucleic acid construct may comprise two or more nucleic acids that are identical to nucleic acids found in nature, or portions thereof, but are not found as part of a single nucleic acid in nature.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

In some embodiments, the agent is encoded by a synthetic RNA (e.g., modified mRNAs). The synthetic RNA can encode any suitable agent described herein. Synthetic RNAs, including modified RNAs are taught in WO 2017075406, which is herein incorporated by reference. In some embodiments, the agent is, or is encoded by, a synthetic RNA (e.g., modified mRNAs) conjugated to non-nucleic acid molecules. In some embodiments, the synthetic RNAs are conjugated to (or otherwise physically associated with) a moiety that promotes cellular uptake, nuclear entry, and/or nuclear retention (e.g., peptide transport moieties or the nucleic acids). In some embodiments, the synthetic RNA is conjugated to a peptide transporter moiety, for example a cell-penetrating peptide transport moiety, which is effective to enhance transport of the oligomer into cells.

In some embodiments, the agent is a targetable nuclease and, if appropriate, a guide molecule (e.g., one or more gRNA). In some embodiments, the agent is capable of making a mutation in a DNA region coding a regRNA (e.g., eRNA) or a DNA region controlling transcription of the regRNA. The term “targetable nuclease” refers to a nuclease that can be programmed to produce site-specific DNA breaks, e.g., double-stranded breaks (DSBs), at a selected site in DNA. Such a site may be referred to as a “target site”. The target site can be selected by appropriate design of the targetable nuclease or by providing a guide molecule (e.g., a guide RNA) directs the nuclease to the target site. Examples of targetable nucleases include zinc finger nucleases (ZFNs), transcription activator-like effector nucleases (TALENs), and RNA-guided nucleases (RGNs) such as the Cas proteins of the CRISPR/Cas Type II system, and engineered meganucleases. In some embodiments, the agent is an RNA-guided nucleases (RGNs) (e.g., the Cas proteins of the CRISPR/Cas Type II system targetable nuclease) and RNA template (e.g., gRNA) capable of making a mutation in a DNA region coding a regRNA (e.g., eRNA) or a DNA region controlling transcription of the regRNA that modulates transcription of the relevant gene.

In some embodiments, the agent comprises an “RNA interference” (RNAi) agent or an antisense oligonucleotide specifically binding with the regRNA.

The term “RNA interference” (RNAi) encompasses processes in which a molecular complex known as an RNA-induced silencing complex (RISC) is formed. RISC may incorporate a short nucleic acid strand (e.g., about 16-about 30 nucleotides (nt) in length) that pairs with RNA (e.g., regRNA, eRNA) to which the strand has complementarity. The short nucleic acid strand may be referred to as a “guide strand” or “antisense strand”. An RNA strand to which the guide strand has complementarity may be referred to as a “target RNA.” A guide strand may initially become associated with RISC components (in a complex sometimes termed the RISC loading complex) as part of a short double-stranded RNA (dsRNA), e.g., a short interfering RNA (siRNA). The other strand of the short dsRNA may be referred to as a “passenger strand” or “sense strand”. The complementarity of the structure formed by hybridization of a target RNA and the guide strand may be such that the strand can (i) guide cleavage of the target RNA in the RNA-induced silencing complex (RISC) and/or (ii) modulate the effective charge, structure, or behavior of the target RNA (e.g., regRNA, eRNA).

In some embodiments, the RNAi agent reduces the amount of regRNA in the condensate by increasing target regRNA cleavage in the RISC. In some embodiments, the RNAi agent modulates the amount of nucleic acid in the condensate. In some embodiments, the RNAi agent binds to and localizes with the target regRNA into the condensate, thereby increasing nucleic acid in the condensate. In some embodiments, the increase in nucleic acid accelerates condensate formation (e.g., increases transcription of the associated gene). In some embodiments, the increase in nucleic acid accelerates condensate dissolution (e.g., decreases transcription of associated gene). In some embodiments, the RNAi agent binds to and modulates the effective charge, structure, or behavior of the regRNA incorporated into the condensate. In some embodiments, the modulation of regRNA effective charge, structure, or behavior accelerates condensate formation (e.g., increases transcription of the associated gene). In some embodiments, the modulation of regRNA effective charge, structure, or behavior accelerates condensate dissolution (e.g., decreases transcription of the associated gene). In some embodiments, the modulation of regRNA effective charge, structure, or behavior stabilizes the condensate (e.g., increases transcription of the associated gene).

As known in the art, the complementarity between the guide strand and a target RNA need not be perfect (100%) but need only be sufficient to result in specific binding to the target RNA. For example, in some embodiments 1, 2, 3, 4, 5, or more nucleotides of a guide strand may not be matched to a target RNA. “Not matched” or “unmatched” refers to a nucleotide that is mismatched (not complementary to the nucleotide located opposite it in a duplex, i.e., wherein Watson-Crick base pairing does not take place) or forms at least part of a bulge. Examples of mismatches include, without limitation, an A opposite a G or A, a C opposite an A or C, a U opposite a Cor U, a G opposite a G. A bulge refers to a sequence of one or more nucleotides in a strand within a generally duplex region that are not located opposite to nucleotide(s) in the other strand. “Partly complementary” refers to less than perfect complementarity. In some embodiments a guide strand has at least about 80%, 85%, or 90%, e.g., at least about 91%, 92%, 93%, 94%, 95%, 96%, 97%, 98%, 99%, or 100% sequence complementarity to a target RNA over a continuous stretch of at least about 15 nt, e.g., between 15 nt and 30 nt, between 17 nt and 29 nt, between 18 nt and 25 nt, between 19 nt and 23 nt, of the target RNA. In some embodiments at least the seed region of a guide strand (the nucleotides in positions 2-7 or 2-8 of the guide strand) is perfectly complementary to a target RNA. In some embodiments, a guide strand and a target RNA sequence may form a duplex that contains no more than 1, 2, 3, or 4 mismatched or bulging nucleotides over a continuous stretch of at least 10 nt, e.g., between 10-30 nt. In some embodiments a guide strand and a target RNA sequence may form a duplex that contains no more than 1, 2, 3, 4, 5, or 6 mismatched or bulging nucleotides over a continuous stretch of at least 12 nt, e.g., between 10-30 nt. In some embodiments, a guide strand and a target RNA sequence may form a duplex that contains no more than 1, 2, 3, 4, 5, 6, 7, or 8 mismatched or bulging nts over a continuous stretch of at least 15 nt, e.g., between 10-30 nt. In some embodiments, a guide strand and a target RNA sequence may form a duplex that contains no mismatched or bulging nucleotides over a continuous stretch of at least 10 nt, e.g., between 10-30 nt. In some embodiments, between 10-30 nt is 10, 11, 12, 13, 14, 15, 16, 17, 18, 19, 20, 21, 22, 23, 24, 25, 26, 27, 28, 29, or 30 nt.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15-about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments, strands may differ by 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., about 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5ÿ with respect to the loop or 3ÿ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., between 19-25 nt. In some embodiments the stem is between 15-19 nt. In some embodiments the stem is between 19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel D P. Cell. 136(2):215-33 (2009); Winter, J., et al., Nature Cell Biology 11: 228-234 (2009). Artificial miRNA may be designed to take advantage of the endogenous RNAi pathway in order to bind with and/or silence a target RNA of interest. The sequence of such artificial miRNA may be selected so that one or more bulges is present when the artificial miRNA is hybridized to its target sequence, mimicking the structure of naturally occurring miRNA:mRNA hybrids. Those of ordinary skill in the art are aware of how to design artificial miRNA.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pre-miRNA).

Antisense oligonucleotides (ASO) are small sequences of DNA or RNA (e.g., about 8-50 base pairs in length) able to target RNA transcripts (e.g., regRNA, eRNA) by Watson-Crick base pairing. In some embodiments, oligonucleotides are unmodified. In other embodiments oligonucleotides include one or more modifications, e.g., to improve solubility, binding, potency, and/or stability of the antisense oligonucleotide. Modified oligonucleotides may comprise at least one modification relative to unmodified RNA or DNA. In some embodiments, oligonucleotides are modified to include internucleoside linkage modifications, sugar modifications, and/or nucleobase modifications. Examples of such modifications are known to those of skill in the art.

In some embodiments the oligonucleotide is modified by the substitution of at least one nucleotide with a modified nucleotide, such that in vivo stability is enhanced as compared to a corresponding unmodified oligonucleotide. In some aspects, the modified nucleotide is a sugar-modified nucleotide. In another aspect, the modified nucleotide is a nucleobase-modified nucleotide.

In some embodiments, oligonucleotides, may contain at least one modified nucleotide analogue. The nucleotide analogues may be located at positions where the target-specific activity, e.g., the splice site selection modulating activity is not substantially affected, e.g., in a region at the 5′-end and/or the 3′-end of the oligonucleotide molecule. In some aspects, the ends may be stabilized by incorporating modified nucleotide analogues.

In some aspects preferred nucleotide analogues include sugar- and/or backbone-modified ribonucleotides (i.e., include modifications to the phosphate-sugar backbone). For example, the phosphodiester linkages of a ribonucleotide may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH2, NHR, NR2 or ON, wherein R is C1-C6 alkyl, alkenyl or alkynyl and halo is F, Cl, Br or I.

In some embodiments, modified oligonucleotides comprise one or more modified nucleosides comprising a modified sugar moiety.

Modified oligonucleotides may comprise one or more nucleosides comprising an unmodified nucleobase. In some embodiments modified oligonucleotides comprise one or more nucleosides comprising a modified nucleobase. In some embodiments, modified oligonucleotides comprise one or more nucleosides that does not comprise a nucleobase.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone.

Also preferred are nucleobase-modified ribonucleotides, i.e., ribonucleotides, containing at least one non-naturally occurring nucleobase instead of a naturally occurring nucleobase. Examples of modified nucleobases include, but are not limited to, uridine and/or cytidine modifications at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine. Oligonucleotide reagents of the invention also may be modified with chemical moieties that improve the in vivo pharmacological properties of the oligonucleotide reagents.

In some embodiments, nucleosides of modified oligonucleotides are linked together using any internucleoside linkage.

Additional modifications are known by those of skill in the art and examples can be found in WO 2019/241648, U.S. Pat. Nos. 10,307,434, 9,045,518, and 10,266,822, each of which is incorporated herein by reference.

Oligonucleotides may be of any size and/or chemical composition sufficient to target a regRNA. In some embodiments, an oligonucleotide is between about 5-300 nucleotides or modified nucleotides. In some aspects an oligonucleotide is between about 10-100, 15-85, 20-70, 25-55, or 30-40 nucleotides or modified nucleotides. In certain aspects an oligonucleotide is between about 15-35, 15-20, 20-25, 25-30, or 30-35 nucleotides or modified nucleotides.

In some embodiments, an oligonucleotide and the target RNA sequence (e.g., regRNA, eRNA) have 100% sequence complementarity. In some aspects, an oligonucleotide may comprise sequence variations, e.g., insertions, deletions, and single point mutations, relative to the target sequence. In some embodiments, an oligonucleotide has at least 70% sequence identity or complementarity to the target RNA. In certain embodiments, an oligonucleotide has at least 70%, 75%, 80%, 85%, 90%, 95%, 97%, 99%, or 100% sequence identity to the target sequence.

In some embodiments, the ASO agent reduces the amount of the target regRNA in the condensate by increasing target regRNA degradation. In some embodiments, the ASO agent modulates the amount of nucleic acid in the condensate. In some embodiments, the ASO agent binds to and localizes with the target regRNA into the condensate, thereby increasing nucleic acid in the condensate. In some embodiments, the increase in nucleic acid accelerates condensate formation (e.g., increases transcription of the associated gene). In some embodiments, the increase in nucleic acid accelerates condensate dissolution (e.g., decreases transcription of associated gene). In some embodiments, the ASO agent binds to and modulates the effective charge, structure, or behavior of the regRNA incorporated into the condensate. In some embodiments, the modulation of regRNA effective charge, structure, or behavior accelerates condensate formation (e.g., increases transcription of the associated gene). In some embodiments, the modulation of regRNA effective charge, structure, or behavior accelerates condensate dissolution (e.g., decreases transcription of the associated gene). In some embodiments, the modulation of regRNA effective charge, structure, or behavior stabilizes the condensate (e.g., increases transcription of the associated gene).

In some embodiments, transcription is modulated by contacting the one or more species of regRNA with an agent (e.g., RNAi agent or ASO agent) capable of specifically binding to the one or more species of regRNA. In some embodiments, the one or more species of regRNA comprises enhancer RNA (eRNA). In some embodiments, the eRNA is a sequence variant associated with a disease or condition.

In some embodiments, the amount, effective charge, structure, or behavior of nucleic acid incorporated into the condensate is modulated by contact with an agent specifically binding to a nucleic acid associated with the condensate. The agent is not limited and may be any suitable agent described herein specifically binding to a nucleic acid associated with the condensate. In some embodiments, the agent is an oligonucleotide (e.g., RNAi agent or ASO agent) specifically binding a target messenger RNA or portion thereof. In some embodiments, the agent reduces the amount of oligonucleotide (e.g., mRNA) associated with the condensate by increasing target oligonucleotide (e.g., mRNA) degradation. In some embodiments, the agent modulates the amount of nucleic acid in the condensate. In some embodiments, the agent binds to and localizes with the target oligonucleotide (e.g., mRNA) into the condensate, thereby increasing nucleic acid in the condensate. In some embodiments, the increase in nucleic acid accelerates condensate formation (e.g., increases transcription of the associated gene). In some embodiments, the increase in nucleic acid accelerates condensate dissolution (e.g., decreases transcription of associated gene). In some embodiments, the ASO agent binds to and modulates the effective charge, structure, or behavior of the oligonucleotide (e.g., mRNA) incorporated into the condensate. In some embodiments, the modulation of oligonucleotide (e.g., mRNA) effective charge, structure, or behavior accelerates condensate formation (e.g., increases transcription of the associated gene). In some embodiments, the modulation of oligonucleotide (e.g., mRNA) effective charge, structure, or behavior accelerates condensate dissolution (e.g., decreases transcription of the associated gene). In some embodiments, the modulation of oligonucleotide (e.g., mRNA) effective charge, structure, or behavior stabilizes the condensate (e.g., increases transcription of the associated gene).

In some embodiments, the condensate dependent transcription of a gene occurs in a cell. The cell is not limited and may be any cell described herein. In some embodiments, the cell is a cancer cell. In some embodiments, the cell is a nerve cell. In some embodiments, the condensate dependent transcription of a gene occurs in vivo in a subject. The subject is not limited. The term “subject” encompasses any vertebrate including but not limited to mammals (e.g., rats, mice, rabbits, sheep, cats, dogs, cows, pigs, and non-human primates), reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

In some embodiments, modulating of condensate dependent transcription of a gene treats, prevents or reduces the likelihood of a disease or condition in the subject. The disease or condition is not limited and may be any disease or condition disclosed herein treatable by modulating condensate dependent transcription of a gene. In some embodiments, modulating of condensate dependent transcription of a gene treats, prevents or reduces the likelihood of a disease or condition associated with a haploinsufficiency. In some embodiments, the disease or condition associated with a haploinsufficiency is a cancer, 1q21.1 deletion syndrome, 5q-syndrome in myelodysplastic syndrome (MDS), 22q11.2 deletion syndrome, CHARGE syndrome, Cleidocranial dysostosis, Ehlers-Danlos syndrome, Frontotemporal dementia caused by mutations in progranulin, GLUT1 deficiency (DeVivo syndrome), Haploinsufficiency of A20, Holoprosencephaly caused by haploinsufficiency in the Sonic Hedgehog gene, Holt-Oram syndrome, Marfan syndrome, Phelan-McDermid syndrome, Polydactyly, or Dravet Syndrome. In some embodiments, modulating of condensate dependent transcription of a gene treats, prevents or reduces the likelihood of a disease or condition associated with gene duplication. In some embodiments, the disease or condition associated with gene duplication is a cancer with an oncogene duplication, Charcot-Marie-Tooth disease type I, or MECP2 duplication syndrome. In some embodiments, modulating of condensate dependent transcription of a gene treats, prevents or reduces the likelihood of a disease or condition associated with an eRNA variant (e.g., an eRNA comprising an SNP). In some embodiments, modulating of condensate dependent transcription of a gene treats, prevents or reduces the likelihood of a disease or condition associated with aberrant transcription (e.g., cancer).

“Treat” as used herein covers any treatment of a disease or condition of a mammal (e.g., cancer, a disease or condition associated with a haploinsufficiency, gene duplication, or an eRNA variant), particularly a human, and includes: (a) preventing symptoms of the disease or condition (e.g., cancer, a disease or condition associated with a haploinsufficiency, gene duplication, or an eRNA variant) from occurring in a subject which may be predisposed to the disease or condition but has not yet begun experiencing symptoms; (b) inhibiting the disease or condition (e.g., arresting its development); or (c) relieving the disease or condition (e.g., causing regression of the disease or condition, providing improvement in one or more symptoms). The method of administration is not limited and may be any suitable method of administration.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, agents may be administered directly to a tissue. Direct tissue administration may be achieved by direct injection. The agents may be administered once, or alternatively they may be administered in a plurality of administrations. If administered multiple times, the peptides may be administered via different routes. For example, the first (or the first few) administrations may be made directly into the affected tissue while later administrations may be systemic.

For oral administration, compositions can be formulated readily by combining the agent with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like In the event that a response in a subject is insufficient at the initial doses applied, higher doses (or effectively higher doses by a different, more localized delivery route) may be employed to the extent that patient tolerance permits. Multiple doses per day are contemplated in some embodiments to achieve appropriate systemic levels of compounds. In some embodiments, the method further includes administering to the subject an effective amount of at least one chemotherapeutic agent. The chemotherapeutic agent is not limited and may be any suitable chemotherapeutic agent known in the art.

RNAi and ASO agents described herein may formulated with one or more acceptable reagents, which provide a vehicle for delivering such to target cells. Appropriate reagents are generally selected with regard to a number of factors, which include, among other things, the biological or chemical properties of the RNAi and ASO agents, the intended route of administration, the anticipated biological environment to which such RNAi and ASO agents will be exposed and the specific properties of the intended target cells. In some embodiments, transfer vehicles, such as liposomes, encapsulate the RNAi and ASO agents without compromising biological activity. In some embodiments, the transfer vehicle demonstrates preferential and/or substantial binding to a target cell relative to non-target cells. In a preferred embodiment, the transfer vehicle delivers its contents to the target cell such that the RNAi and ASO agents are delivered to the appropriate subcellular compartment, such as the cytoplasm.

In some embodiments, the transfer vehicle in the compositions of the invention is a liposomal transfer vehicle, e.g. a lipid nanoparticle. In one embodiment, the transfer vehicle may be selected and/or prepared to optimize delivery of the nucleic acid to a target cell. For example, if the target cell is a hepatocyte the properties of the transfer vehicle (e.g., size, charge and/or pH) may be optimized to effectively deliver such transfer vehicle to the target cell, reduce immune clearance and/or promote retention in that target cell. Alternatively, if the target cell is in the central nervous system (e.g., for the treatment of neurodegenerative diseases, the transfer vehicle may specifically target brain or spinal tissue), selection and preparation of the transfer vehicle must consider penetration of, and retention within the blood brain barrier and/or the use of alternate means of directly delivering such transfer vehicle to such target cell.

The use of liposomal transfer vehicles to facilitate the delivery of nucleic acids to target cells is contemplated by the present disclosure. Liposomes (e.g., liposomal lipid nanoparticles) are generally useful in a variety of applications in research, industry, and medicine, particularly for their use as transfer vehicles of diagnostic or therapeutic compounds in vivo (Lasic, Trends Biotechnol., 16: 307-321, 1998; Drummond et al., Pharmacol. Rev., 51: 691-743, 1999) and are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.).

In the context of the present disclosure, a liposomal transfer vehicle typically serves to transport the nucleic acid to the target cell. For the purposes of the present invention, the liposomal transfer vehicles are prepared to contain the desired nucleic acids. The process of incorporation of a desired RNAi or ASO agent into a liposome is often referred to as “loading” (Lasic, et al., FEBS Lett., 312: 255-258, 1992). The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating a nucleic acid into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in a preferred embodiment of the present invention, the selected transfer vehicle is capable of enhancing the stability of the nucleic acid contained therein. The liposome can allow the encapsulated nucleic acid to reach the target cell and/or may preferentially allow the encapsulated nucleic acid to reach the target cell, or alternatively limit the delivery of such nucleic acid to other sites or cells where the presence of the administered nucleic acid may be useless or undesirable. Furthermore, incorporating the RNAi and ASO agents into a transfer vehicle, such as for example, a cationic liposome, also facilitates the delivery of such into a target cell.

Liposomal transfer vehicles can be prepared to encapsulate one or more desired RNAi and ASO agents such that the compositions demonstrate a high transfection efficiency and enhanced stability. While liposomes can facilitate introduction of nucleic acids into target cells, the addition of polycations (e.g., poly L-lysine and protamine), as a copolymer can facilitate, and in some instances markedly enhance the transfection efficiency of several types of cationic liposomes by 2-28 fold in a number of cell lines both in vitro and in vivo. (See N. J. Caplen, et al., Gene Ther. 1995; 2: 603; S. Li, et al., Gene Ther. 1997; 4, 891.)

In some embodiments, the transfer vehicle is formulated as a lipid nanoparticle. As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more RNAi or ASO agents to one or more target cells.

Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a nucleic acid to a target cell.

The present disclosure contemplates the use of lipid nanoparticles as transfer vehicles comprising a cationic lipid to encapsulate and/or enhance the delivery of nucleic acid into the target cell. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available.

Suitable cationic lipids of use in the compositions and methods herein include those described in international patent publication WO 2010/053572, incorporated herein by reference, e.g., C12-200 described at paragraph of WO 2010/053572. In certain embodiments, the compositions and methods of the invention employ a lipid nanoparticles comprising an ionizable cationic lipid such as, e.g., (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), and (15Z,18Z)—N,N-dimethyl-6-((9Z,12Z)-octadeca-9,12-dien-1-yl)tetracosa-5,15,18-trien-1-amine (HGT5002).

In some embodiments, the cationic lipid N-[1-(2,3-dioleyloxy)propyl]-N,N,N-trimethylammonium chloride or “DOTMA” is used. (Felgner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.′l Acad. Sci. 86, 6982 (1989); U.S. Pat. Nos. 5,171,678; 5,334,761), 1,2-Dioleoyl-3-Dimethylammonium-Propane or “DODAP”, 1,2-Dioleoyl-3-Trimethylammonium-Propane or “DOTAP”. Contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5ÿ-(cholest-5-en-3-beta-oxy)-3ÿ-oxapentoxy)-3-dimethyl-1-(cis,cis-9ÿ, 1-2ÿ-octadecadienoxy)propane or “CpLinDMA”, N,N-dimethyl-3,4-dioleyloxybenzylamine or “DMOBA”, 1,2-N,Nÿ-dioleylcarbamyl-3-dimethylaminopropane or “DOcarbDAP”, 2,3-Dilinoleoyloxy-N,N-dimethylpropylamine or “DLinDAP”, 1,2-N,Nÿ-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-K-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28:172-176 (2010)), or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1).

The use of cholesterol-based cationic lipids is also contemplated by the present disclosure. Such cholesterol-based cationic lipids can be used, either alone or in combination with other cationic or non-cationic lipids. Suitable cholesterol-based cationic lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

The skilled artisan will appreciate that various reagents are commercially available to enhance transfection efficacy. Suitable examples include LIPOFECTIN (DOTMA:DOPE) (Invitrogen, Carlsbad, Calif.), LIPOFECTAMINE (DOSPA:DOPE) (Invitrogen), LIPOFECTAMINE2000. (Invitrogen), FUGENE, TRANSFECTAM (DOGS), and EFFECTENE.

Also contemplated are cationic lipids such as the dialkylamino-based, imidazole-based, and guanidinium-based lipids. For example, certain embodiments are directed to a composition comprising one or more imidazole-based cationic lipids, for example, the imidazole cholesterol ester or “ICE” lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate. In a preferred embodiment, a transfer vehicle for delivery of synthetic RNA (e.g., modified mRNA) may comprise one or more imidazole-based cationic lipids, for example, the imidazole cholesterol ester or “ICE” lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate.

The imidazole-based cationic lipids are also characterized by their reduced toxicity relative to other cationic lipids. The imidazole-based cationic lipids (e.g., ICE) may be used as the sole cationic lipid in the lipid nanoparticle, or alternatively may be combined with traditional cationic lipids, non-cationic lipids, and PEG-modified lipids. The cationic lipid may comprise a molar ratio of about 1% to about 90%, about 2% to about 70%, about 5% to about 50%, about 10% to about 40% of the total lipid present in the transfer vehicle, or preferably about 20% to about 70% of the total lipid present in the transfer vehicle.

In some embodiments, the lipid nanoparticles comprise the HGT4003 cationic lipid 2-((2,3-Bis((9Z,12Z)-octadeca-9,12-dien-1-yloxy)propyl)disulfanyl)-N,N-dimethylethanamine, as further described in US Pub. No. 20140288160 the entire teachings of which are incorporated herein by reference in their entirety.

In other embodiments the compositions and methods described herein are directed to lipid nanoparticles comprising one or more cleavable lipids, such as, for example, one or more cationic lipids or compounds that comprise a cleavable disulfide (S—S) functional group (e.g., HGT4001, HGT4002, HGT4003, HGT4004 and HGT4005), as further described in US Pub. No. 20140288160, the entire teachings of which are incorporated herein by reference in their entirety.

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipids together which comprise the transfer vehicle (e.g., a lipid nanoparticle). Contemplated PEG-modified lipids include, but is not limited to, a polyethylene glycol chain of up to 5 kDa in length covalently attached to a lipid with alkyl chain(s) of C6-C20 length. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613). In some embodiments, exchangeable lipids comprise PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle.

The present disclosure also contemplates the use of non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone, but are preferably used in combination with other excipients, for example, cationic lipids. When used in combination with a cationic lipid, the non-cationic lipid may comprise a molar ratio of 5% to about 90%, or preferably about 10% to about 70% of the total lipid present in the transfer vehicle.

In some embodiments, the transfer vehicle (e.g., a lipid nanoparticle) is prepared by combining multiple lipid and/or polymer components. For example, a transfer vehicle may be prepared using C12-200, DOPE, chol, DMG-PEG2K at a molar ratio of 40:30:25:5, or DODAP, DOPE, cholesterol, DMG-PEG2K at a molar ratio of 18:56:20:6, or HGT5000, DOPE, chol, DMG-PEG2K at a molar ratio of 40:20:35:5, or HGT5001, DOPE, chol, DMG-PEG2K at a molar ratio of 40:20:35:5. The selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the synthetic RNA (e.g., modified mRNA) to be delivered. Additional considerations include, for example, the saturation of the alkyl chain, as well as the size, charge, pH, pKa, fusogenicity and toxicity of the selected lipid(s). Thus the molar ratios may be adjusted accordingly. For example, in embodiments, the percentage of cationic lipid in the lipid nanoparticle may be greater than 10%, greater than 20%, greater than 30%, greater than 40%, greater than 50%, greater than 60%, or greater than 70%. The percentage of non-cationic lipid in the lipid nanoparticle may be greater than 5%, greater than 10%, greater than 20%, greater than 30%, or greater than 40%. The percentage of cholesterol in the lipid nanoparticle may be greater than 10%, greater than 20%, greater than 30%, or greater than 40%. The percentage of PEG-modified lipid in the lipid nanoparticle may be greater than 1%, greater than 2%, greater than 5%, greater than 10%, or greater than 20%.

In certain embodiments, the lipid nanoparticles of the present disclosure comprise at least one of the following cationic lipids: C12-200, DLin-KC2-DMA, DODAP, HGT4003, ICE, HGT5000, or HGT5001. In embodiments, the transfer vehicle comprises cholesterol and/or a PEG-modified lipid. In some embodiments, the transfer vehicles comprises DMG-PEG2K. In certain embodiments, the transfer vehicle comprises one of the following lipid formulations: C12-200, DOPE, chol, DMG-PEG2K; DODAP, DOPE, cholesterol, DMG-PEG2K; HGT5000, DOPE, chol, DMG-PEG2K, HGT5001, DOPE, chol, DMG-PEG2K.

The liposomal transfer vehicles for use in the compositions of the disclosure can be prepared by various techniques which are presently known in the art. Multi-lamellar vesicles (MLV) may be prepared conventional techniques, for example, by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques.

In certain embodiments, the compositions of the present disclosure comprise a transfer vehicle wherein the RNAi and ASO agent is associated on both the surface of the transfer vehicle and encapsulated within the same transfer vehicle. For example, during preparation of the compositions of the present invention, cationic liposomal transfer vehicles may associate with the agent through electrostatic interactions.

In certain embodiments, the compositions of the invention may be loaded with diagnostic radionuclide, fluorescent materials or other materials that are detectable in both in vitro and in vivo applications. For example, suitable diagnostic materials for use in the present invention may include Rhodamine-dioleoylphospha-tidylethanolamine (Rh-PE), Green Fluorescent Protein mRNA (GFP mRNA), Renilla Luciferase mRNA and Firefly Luciferase mRNA.

Selection of the appropriate size of a liposomal transfer vehicle may take into consideration the site of the target cell or tissue and to some extent the application for which the liposome is being made. In some embodiments, it may be desirable to limit transfection of the RNAi or ASO agent to certain cells or tissues. For example, to target hepatocytes a liposomal transfer vehicle may be sized such that its dimensions are smaller than the fenestrations of the endothelial layer lining hepatic sinusoids in the liver; accordingly the liposomal transfer vehicle can readily penetrate such endothelial fenestrations to reach the target hepatocytes. Alternatively, a liposomal transfer vehicle may be sized such that the dimensions of the liposome are of a sufficient diameter to limit or expressly avoid distribution into certain cells or tissues. For example, a liposomal transfer vehicle may be sized such that its dimensions are larger than the fenestrations of the endothelial layer lining hepatic sinusoids to thereby limit distribution of the liposomal transfer vehicle to hepatocytes. Generally, the size of the transfer vehicle is within the range of about 25 to 250 nm, preferably less than about 250 nm, 175 nm, 150 nm, 125 nm, 100 nm, 75 nm, 50 nm, 25 nm or 10 nm.

A variety of alternative methods known in the art are available for sizing of a population of liposomal transfer vehicles. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

Some aspects of the present disclosure are directed to a method of treating, preventing or reducing the likelihood of a disease or condition associated with aberrant condensate dependent transcription of a gene in a subject comprising administering to the subject an agent that modulates an amount, effective charge, structure, or behavior of nucleic acid in the condensate and thereby modulates transcription of the gene. The subject is not limited and may be any subject disclosed herein. In some embodiments, the subject is human.

The disease or condition associated with aberrant condensate dependent transcription is not limited and may be any suitable gene described herein. In some embodiments, modulating aberrant condensate dependent transcription of a gene treats, prevents or reduces the likelihood of a disease or condition associated with a haploinsufficiency. In some embodiments, the disease or condition associated with a haploinsufficiency is a cancer, 1q21.1 deletion syndrome, 5q-syndrome in myelodysplastic syndrome (MDS), 22q11.2 deletion syndrome, CHARGE syndrome, Cleidocranial dysostosis, Ehlers-Danlos syndrome, Frontotemporal dementia caused by mutations in progranulin, GLUT1 deficiency (De Vivo syndrome), Haploinsufficiency of A20, Holoprosencephaly caused by haploinsufficiency in the Sonic Hedgehog gene, Holt-Oram syndrome, Marfan syndrome, Phelan-McDermid syndrome, Polydactyly, or Dravet Syndrome. In some embodiments, modulating aberrant condensate dependent transcription of a gene treats, prevents or reduces the likelihood of a disease or condition associated with gene duplication. In some embodiments, the disease or condition associated with gene duplication is a cancer with an oncogene duplication, Charcot-Marie-Tooth disease type I, or MECP2 duplication syndrome. In some embodiments, the disease or condition is associated with an eRNA variant.

In some embodiments, the condensate dependent transcription occurs in the presence of one or more species of regulatory RNA (regRNA) in the condensate. The regRNA is not limited and may be any regRNA disclosed herein. In some embodiments, the regRNA is an eRNA. In some embodiments, the regRNA (e.g., eRNA) comprises an SNP. In some embodiments, the regRNA (e.g., eRNA) is associated with a gene described herein (e.g., a gene associated with a disease or condition described herein).

The agent is not limited and may be any agent described herein. In some embodiments, the agent comprises a nucleic acid having a sequence complementary to a sequence of the one or more species of regRNA. In some embodiments, the agent is an RNAi agent or ASO agent as described herein.

Some aspects of the present disclosure are directed towards a method of treating, preventing or reducing the likelihood of a disease or condition in a subject comprising administering to the subject an agent that modulates an amount, effective charge, structure, or behavior of nucleic acid in a transcriptional condensate in a cell of the subject and thereby modulates transcription of a gene and treats, prevents, or reduces the likelihood of the disease or condition. The subject is not limited and may be any subject disclosed herein. In some embodiments, the subject is human. In some embodiments, the method increases transcription of the gene or a level of gene product in the subject. In some embodiments, the transcription of the gene or a level of gene product is increased by less than 2-fold. In some embodiments, the method decreases transcription of the gene or a level of gene product. In some embodiments, transcription of the gene or a level of gene product is decreased by less than 50%.

The agent is not limited and may be any suitable agent described herein. In some embodiments, the agent specifically associates with or binds to a nucleic acid associated with the condensate. In some embodiments, the agent is an RNAi agent or ASO agent. The nucleic acid associated with the condensate is not limited. In some embodiments, the nucleic acid is a regRNA (e.g., eRNA). In some embodiments, the regRNA (e.g., eRNA) is associated with a gene described herein (e.g., a gene associated with a disease or condition described herein) or the regRNA has an SNP and is associated with a disease or condition. In some embodiments, the nucleic acid is an mRNA.

### Methods of Screening

Some aspects of the present disclosure are related to a method of identifying an agent that modulates a condensate, comprising providing a condensate comprising a regulatory RNA (regRNA), contacting the condensate with a test agent, and assessing whether the test agent dissolves or modulates the size of the condensate.

The condensate is not limited and may be any suitable condensate described herein. In some embodiments, the condensate is in a cell (e.g., a transgenic cell). In some embodiments, the condensate is isolated from a cell. In some embodiments, the condensate is a synthetic condensate. In some embodiments, the condensate is a transcriptional condensate or a synthetic transcriptional condensate. In some embodiments, the condensate is capable of transcribing a reporter gene. The components of the condensate may be any condensate component described herein.

The regRNA is not limited and may be any regRNA described herein. In some embodiments, the regRNA is an enhancer RNA. In some embodiments, the regRNA (e.g., eRNA) comprises an SNP associated with a disease or condition. In some embodiments, the regRNA (e.g., eRNA) is associated with a disease or condition or associated with a gene associated with a disease or condition (e.g., as described herein). The disease or condition is not limited and may be any disease or condition described herein (e.g., cancer, a disease or condition associated with haploinsufficiency, gene duplication, or eRNA variant).

Some aspects of the present disclosure are related to a method of identifying an agent that increases condensate formation, comprising providing a composition comprising a regulatory RNA (regRNA) and a condensate component under conditions wherein the concentration of the regRNA or condensate component does not form a condensate, contacting the composition with a test agent, and assessing whether contact with the test agent causes formation of a condensate.

The condensate is not limited and may be any suitable condensate described herein. In some embodiments, the condensate is in a cell (e.g., a transgenic cell). In some embodiments, the condensate is isolated from a cell. In some embodiments, the condensate is a synthetic condensate. In some embodiments, the condensate is a transcriptional condensate or a synthetic transcriptional condensate. In some embodiments, the condensate is capable of transcribing a reporter gene. The components of the condensate may be any condensate component described herein.

The regRNA is not limited and may be any regRNA described herein. In some embodiments, the regRNA is an enhancer RNA. In some embodiments, the regRNA (e.g., eRNA) comprises an SNP associated with a disease or condition. In some embodiments, the regRNA (e.g., eRNA) is associated with a disease or condition or associated with a gene associated with a disease or condition. The disease or condition is not limited and may be any disease or condition described herein (e.g., cancer, a disease or condition associated with haploinsufficiency, gene duplication, or eRNA variant).

Some aspects of the present disclosure are directed to a method of identifying an agent that modulates condensate dependent transcription of a gene, comprising providing an in vitro transcription assay with condensate dependent expression of a reporter gene, contacting the in vitro transcription assay with a test agent, and assessing expression of the reporter gene, wherein condensate dependent expression requires incorporation of a regulatory RNA (regRNA) in the condensate.

The condensate is not limited and may be any suitable condensate described herein. In some embodiments, the condensate is isolated from a cell. In some embodiments, the condensate is a synthetic condensate. The components of the condensate may be any suitable condensate component described herein.

The regRNA is not limited and may be any regRNA described herein. In some embodiments, the regRNA is an enhancer RNA. In some embodiments, the regRNA (e.g., eRNA) comprises an SNP associated with a disease or condition. In some embodiments, the regRNA (e.g., eRNA) is associated with a disease or condition or associated with a gene associated with a disease or condition. The disease or condition is not limited and may be any disease or condition described herein (e.g., cancer, a disease or condition associated with haploinsufficiency, gene duplication, or eRNA variant).

Some aspects of the present disclosure are related to a method of identifying an agent that modulates condensate dependent transcription of a gene, comprising providing a cell with condensate dependent expression of a heterologous reporter gene, contacting the cell with a test agent, and assessing expression of the heterologous reporter gene, wherein condensate dependent expression requires incorporation of a regulatory RNA (regRNA) in the condensate. The cell is not limited and may be any cell described herein. In some embodiments, the cell is a cancer cell or a diseased cell (e.g., a cell exhibiting a disease described herein).

The regRNA is not limited and may be any regRNA described herein. In some embodiments, the regRNA is an enhancer RNA. In some embodiments, the regRNA (e.g., eRNA) comprises an SNP associated with a disease or condition. In some embodiments, the regRNA (e.g., eRNA) is associated with a disease or condition or associated with a gene associated with a disease or condition. The disease or condition is not limited and may be any disease or condition described herein (e.g., cancer, a disease or condition associated with haploinsufficiency, gene duplication, or eRNA variant).

In some embodiments of the screening methods disclosed herein, the condensate has a detectable tag. The detectable tag can be used to determine if contact with the test agent modulates formation, stability, or morphology of the condensate. In some embodiments, a cell is a genetically engineered to express the detectable tag. In some embodiments, the detectable tag is incorporated into the condensate (e.g., a component of the condensate, the regRNA). The term “detectable tag” or “detectable label” as used herein includes, but is not limited to, detectable labels, such as fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-tag, biotin; fluorescence quenchers typically used in conjunction with a fluorescent tag on the other polypeptide; nucleic acid intercalating agents, and complementary bioluminescent or fluorescent polypeptide fragments. A tag that is a detectable label or a complementary bioluminescent or fluorescent polypeptide fragment may be measured directly (e.g., by measuring fluorescence or radioactivity of, or incubating with an appropriate substrate or enzyme to produce a spectrophotometrically detectable color change for the associated polypeptides as compared to the unassociated polypeptides). A tag that is a heterologous epitope or ligand is typically detected with a second component that binds thereto, e.g., an antibody or binding protein, wherein the second component is associated with a detectable label.

In some specific embodiments of the screening methods disclosed herein, the condensate comprises a mediator component or fragment thereof comprising an IDR. In some embodiments, the mediator component or fragment thereof comprising an IDR further comprises a label.

In some embodiments of the screening methods disclosed herein, “assessing” comprises measuring a physical property as compared to a control or reference. For example, assessing if the condensate is dissolved or the stability of a condensate is modulated may comprise measuring the period of time a condensate exists as compared to a control condensate not subject to a test condition or agent. assessing if the shape or size of a condensate is modulated can comprise comparing the shape of a condensate as compared to a control condensate not subject to a test condition or agent. In some embodiments, one or more properties of a condensate may be “assessed” to be modulated if they are changed by a statistically significant amount (e.g., at least 10%, at least 20%, at least 30%, at least 50%, at least 75%, or more).

In some embodiments of the screening methods disclosed herein, the step of determining if contact with the test agent modulates size, dissolution, formation, stability, or morphology of the condensate is performed using microscopy, which is not limited. In some embodiments, the microscopy is deconvolution microscopy, structured illumination microscopy, or interference microscopy. In some embodiments, the step of determining if contact with the test agent modulates formation, stability, or morphology of the condensate is performed using DNA-FISH, RNA-FISH, or a combination thereof.

In some embodiments of the screening methods disclosed herein, the cell or condensate does not express a reporter gene prior to contact with a test agent and expresses a reporter gene after contact with an agent that enhances condensate formation, stability, size, function, or morphology. In some embodiments, the cell does express a reporter gene prior to contact with a test agent and stops or reduces expression of the reporter gene after contact with an agent that dissolves condensates, reduces condensate stability, or prevents/supresses condensate formation.

In some embodiments of the screening methods disclosed herein, a high throughput screen (HTS) is performed. A high throughput screen can utilize cell-free or cell-based assays (e.g., a condensate containing cell as described herein, an in vitro condensate, an isolated in vitro condensate). High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. For example, a candidate anticancer agent may be tested against multiple different cancer cell lines or in an in vivo tumor model to determine its effect on cancer cell survival or proliferation, tumor growth, etc. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminated such unfavorable characteristic(s).

In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. In some embodiments a molecular target of a hit compound is identified or known. In some embodiments, additional compounds that act on the same molecular target may be identified empirically (e.g., through screening a compound library) or designed.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics

Once a candidate agent is identified, additional agents, e.g., analogs, may be generated based on it. An additional agent, may, for example, have increased cancer cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

The test agent for the screening methods disclosed herein are not limited and may be a type of agent described herein (e.g., a protein, nucleic acid, small molecule, etc.) In some embodiments of the screening methods disclosed herein, the test agent is designed to specifically binds the regRNA (e.g., an RNAi agent or ASO agent).

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated.”

### EXAMPLES

Recent studies have shown that transcriptional condensates can compartmentalize and concentrate large numbers of transcription factors, cofactors and Pol II at super-enhancers, which are clusters of enhancers that regulate genes with prominent roles in cell identity (Boija et al., 2018; Cho et al., 2018; Cramer, 2019; Hnisz et al., 2017; Sabari et al., 2018). The component enhancer elements of such genes promote transcriptional condensate formation by crowding transcription factors and Mediator at densities above sharply defined thresholds for condensate formation (Shrinivas et al., 2019). Transcriptional condensates are highly dynamic and can be observed in live cells to form and dissolve at timescales ranging from seconds to minutes (Cho et al., 2018).

RNA molecules are components of, and play regulatory roles in, diverse biomolecular condensates. These include the nucleolus, nuclear speckles, paraspeckles, and stress granules (Fay and Anderson, 2018; Roden and Gladfelter, 2020; Sabari et al., 2020; Strom and Brangwynne, 2019). RNA has a high negative charge density due to its phosphate backbone, and the effective charge of a given RNA molecule is directly proportional to its length (Boeynaems et al., 2019). Condensates are thought to be formed by an ensemble of low-affinity molecular interactions, including electrostatic interactions, and RNA can be a powerful regulator of condensates that are formed and maintained by electrostatic forces (Banani et al., 2017; Maharana et al., 2018; Peran and Mittag, 2020; Shin and Brangwynne, 2017). Indeed, RNA has been shown to enter and modify the properties of simple condensates formed by polyelectrolyte-rich molecules (Drobot et al., 2018; Frankel et al., 2016; Mountain and Keating, 2020). In a phenomenon called complex coacervation, a type of liquid-liquid phase separation mediated by electrostatic interactions between oppositely charged polyelectrolytes, low levels of RNA can enhance condensate formation whereas high levels can cause their dissolution (Lin et al., 2019; Overbeek and Voorn, 1957; Sing, 2017; Srivastava and Tirrell, 2016). Condensate formation and subsequent dissolution with increasing RNA concentration is an example of reentrant phase behavior, which is driven by favorable opposite-charge interactions at low RNA concentrations (formation) and repulsive like-charge interactions at high RNA concentrations (dissolution) (Banerjee et al., 2017; Milin and Deniz, 2018). The inventors investigated whether such a reentrant equilibrium phase behavior coupled to the non-equilibrium processes that occur during transcription could regulate transcriptional output.

By combining physics-based modeling and experimental analysis, the inventors have proposed and tested a model whereby the products of transcription initiation stimulate condensate formation and those of a burst of elongation stimulate condensate dissolution. The inventors provide experimental evidence that physiological RNA levels can enhance or dissolve transcriptional condensates. These results provide a mechanism by which the products of transcription regulate condensate behaviors and thus transcription, and suggest that this non-equilibrium process provides negative feedback to dissolve the transcriptional condensates that support initiation and thereby arrest transcription.

### Results

### Low Levels of RNA Enhance and High Levels Dissolve Mediator Condensates

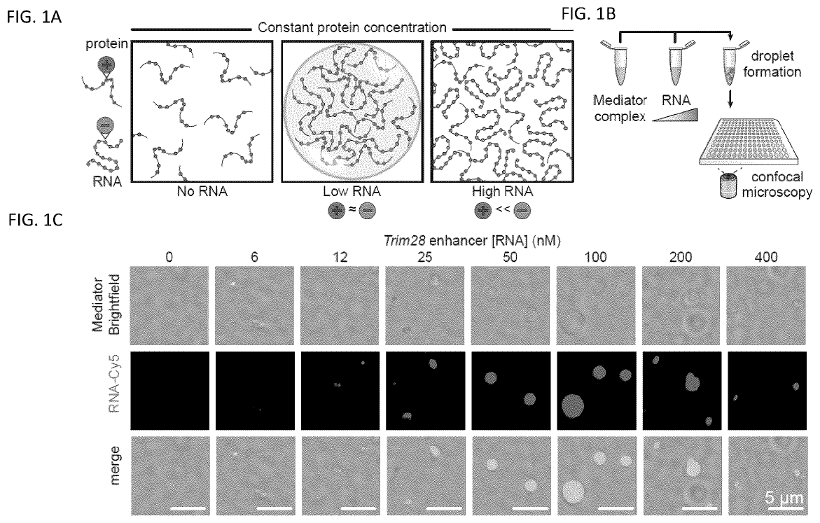

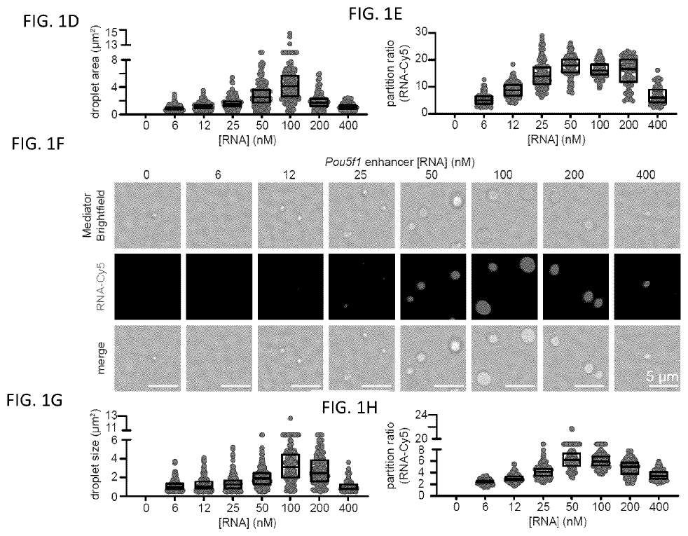

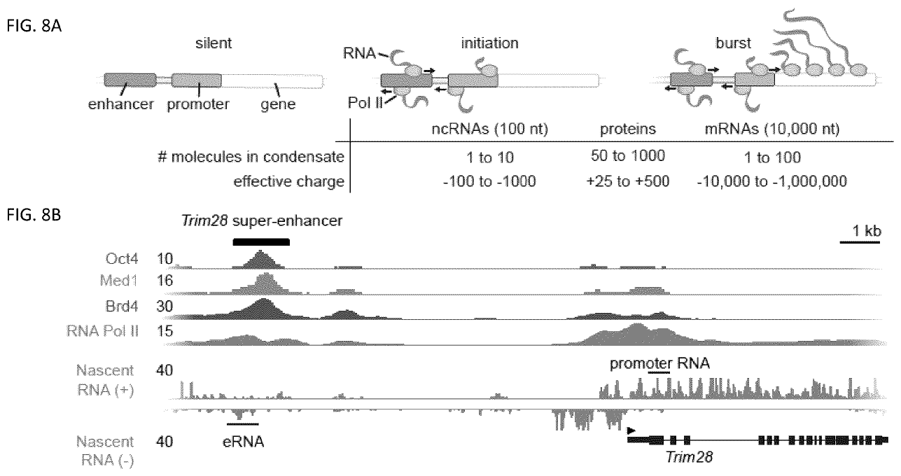

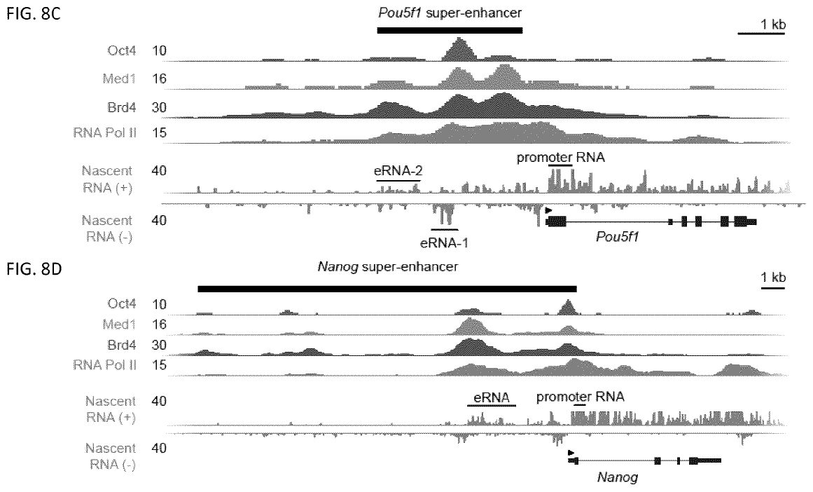

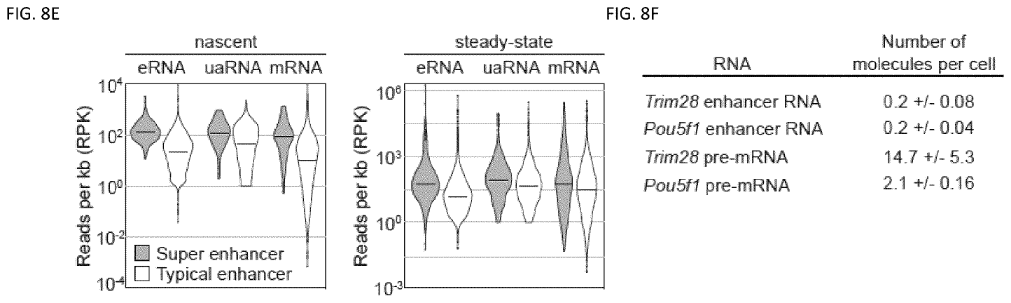

To explore the potential role of RNA in regulating transcriptional condensates, the inventors sought to estimate the number and effective charge of RNA and protein molecules in a typical transcriptional condensate at different stages of transcription. In early stages of transcription, low levels of small noncoding RNAs are produced by Pol II at enhancers and promoter-proximal regions (FIG. 8A) (Adelman and Lis, 2012; Core and Adelman, 2019; Kim et al., 2010; Seila et al., 2008). During pause release, Pol II produces longer genic RNAs during bursts of transcription elongation (FIG. 8A) (Adelman and Lis, 2012; Core and Adelman, 2019). These protein- and RNA-rich states can be thought of as mixtures of poly-electrolytes that may undergo complex coacervation (FIG. 1A) (Lin et al., 2019; Overbeek and Voorn, 1957; Sing, 2017; Srivastava and Tirrell, 2016). This is likely to be relevant to transcriptional condensates because electrostatic interactions contribute to the formation of these condensates, even in the absence of RNA (Boija et al., 2018; Sabari et al., 2020). Complex coacervate formation through phase separation is promoted when poly-electrolytes are present at concentrations where their net charges are approximately balanced. When the concentration of a poly-electrolyte, such as RNA, becomes sufficiently high, the domination of repulsive like-charge interactions can suppress phase separation (Banerjee et al., 2017; Lin et al., 2019; Milin and Deniz, 2018; Muthukumar, 2016; Overbeek and Voorn, 1957; Zhang et al., 2018). Thus, at constant protein concentration, titrating RNA levels results in reentrant phase behavior, by which low RNA levels promote and high RNA levels suppress condensate formation (FIG. 1A) (Banerjee et al., 2017; Milin and Deniz, 2018; Zhang et al., 2018). The inventors wondered whether the reentrant phase behavior might apply to the regulation of transcriptional condensates during transcription. Because the quantities of the diverse RNA species and proteins present in transcriptional condensates in populations of cells can be estimated (FIGS. 8A-8F, STAR Methods), it is possible to conduct experimental tests to determine whether reentrant phase behavior occurs under physiologically-relevant conditions of these molecules.

As an initial test of whether low levels of RNA stimulate transcriptional condensate formation while high levels of RNA favor condensate dissolution, the inventors used an in vitro droplet assay (FIG. 1B). Using components at physiologically-relevant conditions, the inventors investigated whether an enhancer RNA transcribed from the Trim28 super-enhancer, which has previously been shown to form a transcriptional condensate in living cells (Boija et al., 2018; Guo et al., 2019), influences condensate formation by purified Mediator complex. Measurement of enhancer RNA levels in cells indicated that ˜0.2 molecules of this enhancer RNA exist at steady-state in murine embryonic stem cells (mESCs) (FIG. 8F). Given that multiple loci in a super-enhancer are transcribed into enhancer RNAs, this roughly corresponds to ˜100-1000 nM of RNA in a typical Mediator condensate in cells (STAR Methods). These condensates typically contain Mediator at a concentration of around 1-20 μM (STAR Methods). The results showed that addition of 6-400 nM Trim28 enhancer RNA to 200 nM purified Mediator complex had a dose-dependent effect on the size of Mediator/RNA droplets (FIGS. 1C-1E). Droplet sizes peaked at 100 nM RNA (FIG. 1D) and the relative enrichment of RNA in the droplets, as measured by the ratio of average intensity inside versus outside the droplet (partition ratio), followed a similar trend (FIG. 1E). Similar results were obtained using an enhancer RNA transcribed from the Pou5f1 super-enhancer (FIGS. 1F-1H). Thus, within the range of physiological levels observed in cells, low levels of RNA can enhance condensate formation and high levels of RNA can reduce condensate formation by Mediator in vitro.

### RNA-Mediated Regulation of MED1-IDR Condensates Fits a Charge Balance Model

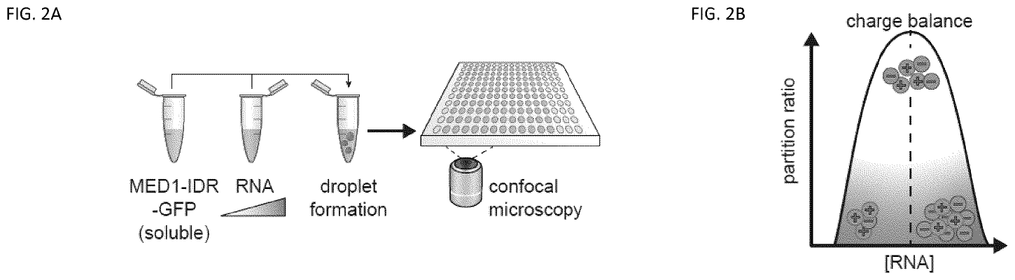

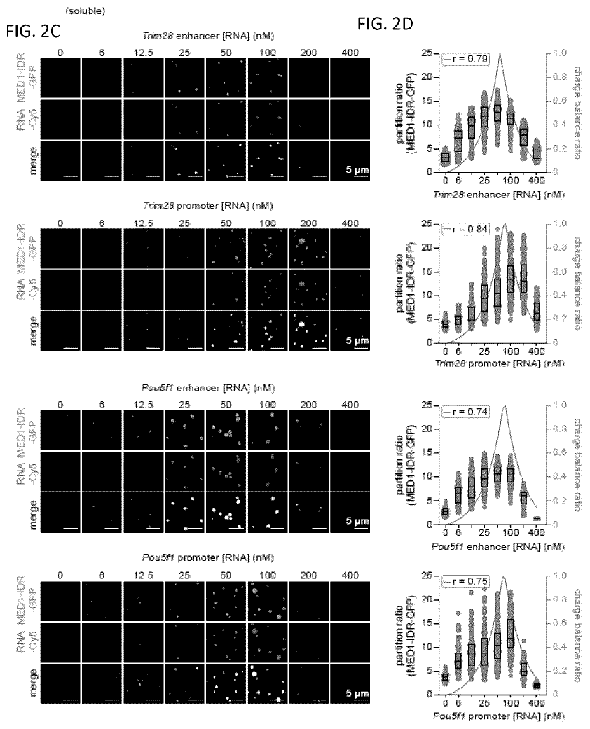

The inventors next sought to quantify how diverse RNAs regulate the reentrant phase behavior of transcriptional proteins. The inventors performed in vitro droplet assays (FIG. 2A) using the MED1 C-terminal intrinsically disordered region (MED1-IDR), which has proven to be a useful surrogate for the multisubunit Mediator complex, as it is not possible to purify sufficient amounts of this complex to test all the parameters of interest (Boija et al., 2018; Guo et al., 2019; Klein et al., 2020; Li et al., 2020; Sabari et al., 2018; Shrinivas et al., 2019; Zamudio et al., 2019). The fusion of GFP to MED1-IDR allows quantification by fluorescence of a single species whose effective charge can be calculated to determine the charge ratio between protein and RNA. Addition of increasing levels of RNA to a constant protein concentration should have predictable effects on the partitioning of either component according to their charge ratio (FIG. 2B). Noncoding and coding RNAs produced from three different super-enhancer loci and their associated genes (Trim28, Pou5f1, Nanog;

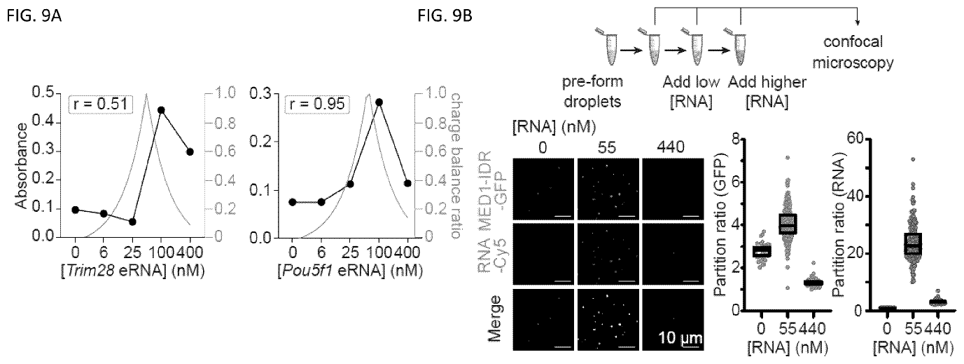

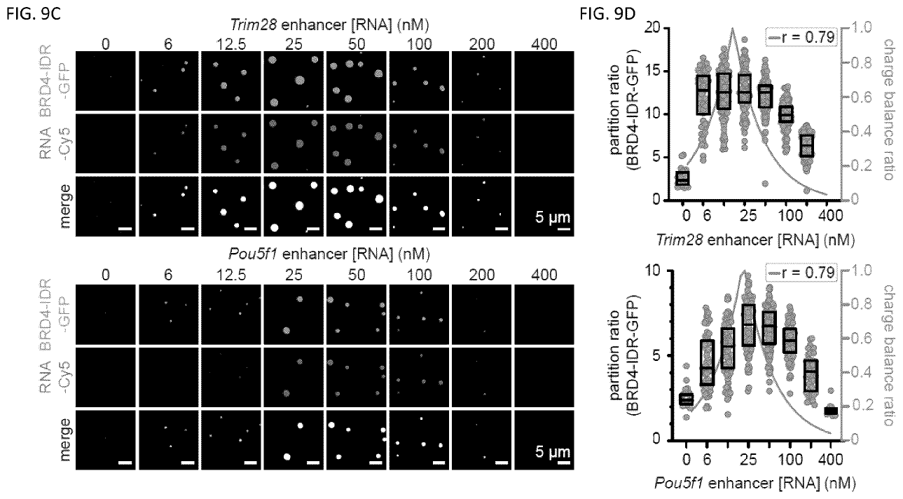

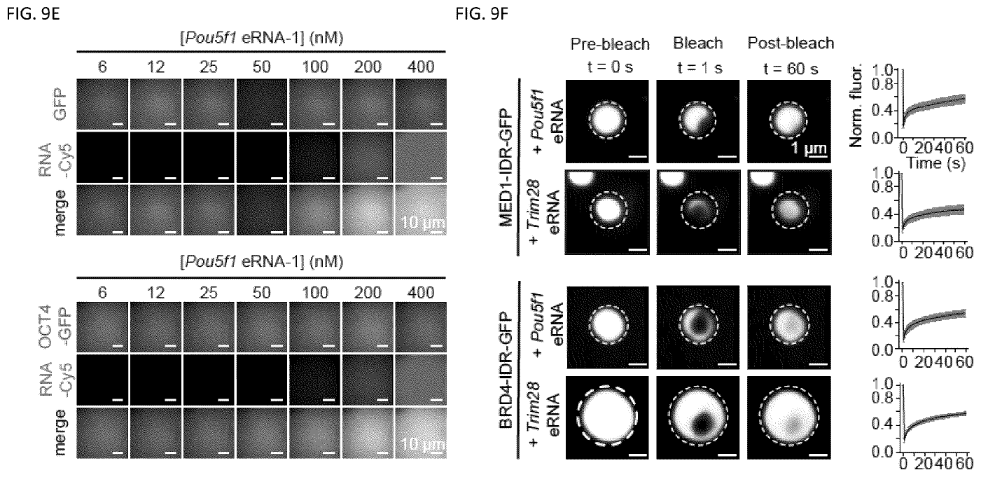

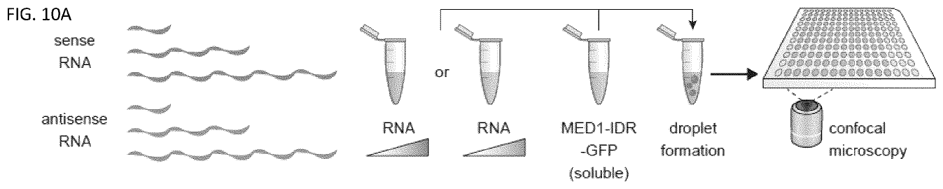

FIG. 8) were selected for this analysis based on prior studies of nascent RNA sequencing data in mESCs (Boija et al., 2018; Guo et al., 2019; Sabari et al., 2018; Sigova et al., 2015; Whyte et al., 2013). Addition of 6-400 nM of each of these RNAs to 1000 nM MED1-IDR (protein: RNA ratios=167 to 2.5) stimulated formation of MED1-IDR condensates at low RNA concentrations and dissolved MED1-IDR condensates at higher RNA concentrations (FIGS. 2C, 2D, 8A, and 8B). BRD4 is another key component of transcriptional condensates and BRD4-IDR protein exhibits condensate behaviors very similar to those of MED1-IDR (Sabari et al., 2018); the effects of increasing RNA levels on formation and dissolution of BRD4-IDR condensates were very similar to those observed for MED1-IDR (FIGS. 9C and 9D). RNA did not stimulate formation of droplets with GFP alone or OCT4-GFP, both of which have a net negative charge (FIG. 9E). Condensates exhibited internal dynamical reorganization (FIG. 9F) with apparent diffusion coefficients 3-5±0.8×10⁻² μm²/s (STAR Methods), consistent with liquid-like behavior (Nott et al., 2015; Sabari et al., 2018; Taylor et al., 2019). These results show that diverse RNAs are capable of stimulating MED1-IDR condensate formation when present at relatively low levels and dissolving MED1-IDR condensates at high levels.

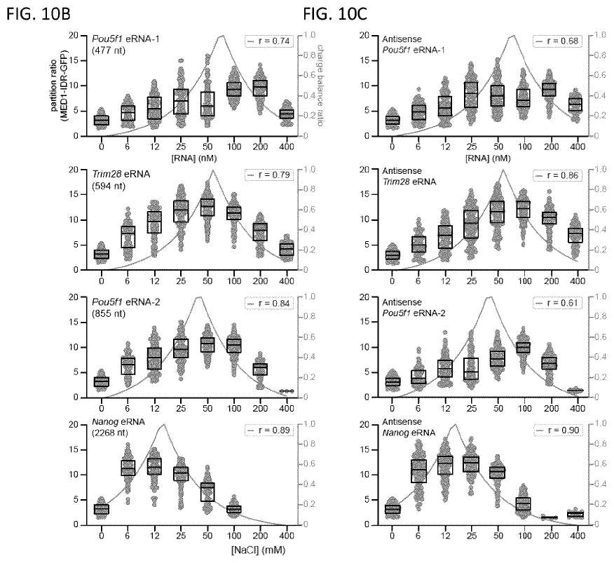

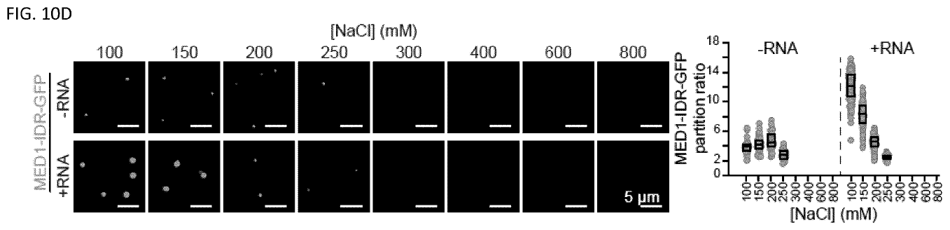

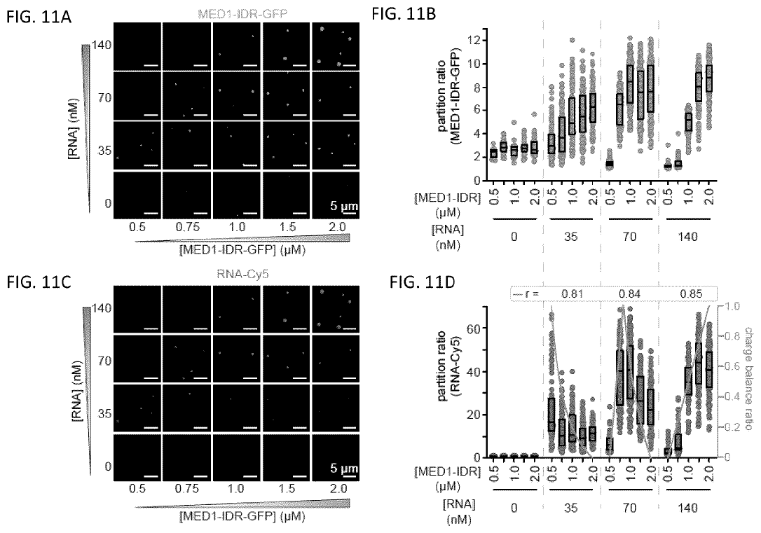

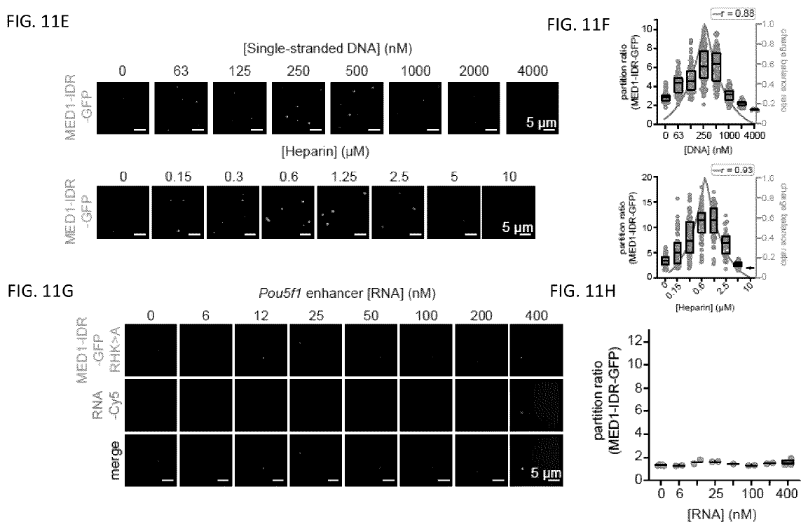

The inventors sought to further test whether the RNA-mediated effects on MED1-IDR condensates fit a charge balance model (STAR Methods). MED1-IDR/RNA condensate formation should be enhanced when the protein and RNA polymers are balanced in charge, and they should be sensitive to disruption of this balance. To test this model, the inventors quantified the relative charge of RNA and MED1-IDR and computed the correlation with the partition ratio of MED1-IDR (STAR Methods). As expected, RNA-mediated effects on MED1-IDR condensates fit a charge balance model (FIG. 2D). The inventors would expect an RNA length-dependent shift in the RNA level required for peak MED1-IDR partitioning when RNAs of different length are introduced into the droplet assay in equal numbers. This expectation that a higher concentration of shorter RNAs is needed to disrupt condensate formation was observed (FIGS. 10A and 10B). Another prediction of the charge balance model is that these interactions should be largely independent of RNA sequence, so antisense versions of any one of the RNA species should exhibit the same quantitative effects as the sense strand, and this was also observed (FIGS. 10B and 10C). As expected for a charge-balance model, MED1-IDR condensates formed with RNA were sensitive to increasing monovalent salt, which screens charged interactions (FIG. 10D). The charge balance model also held when MED1-IDR and RNA concentrations were varied (FIGS. 11A-11D), and when alternative polyanions (heparin and ssDNA) were employed (FIGS. 11E and 11F). RNA did not stimulate condensate formation by a MED1-IDR mutant lacking positively-charged residues (MED1-IDR RHK>A) (FIGS. 11G and 11H). These results further support a charge balance model for the RNA-mediated effects on the equilibrium behavior of MED1-IDR condensates.

### RNA-Mediated Effects on Condensates in Reconstituted In Vitro Transcription Assays

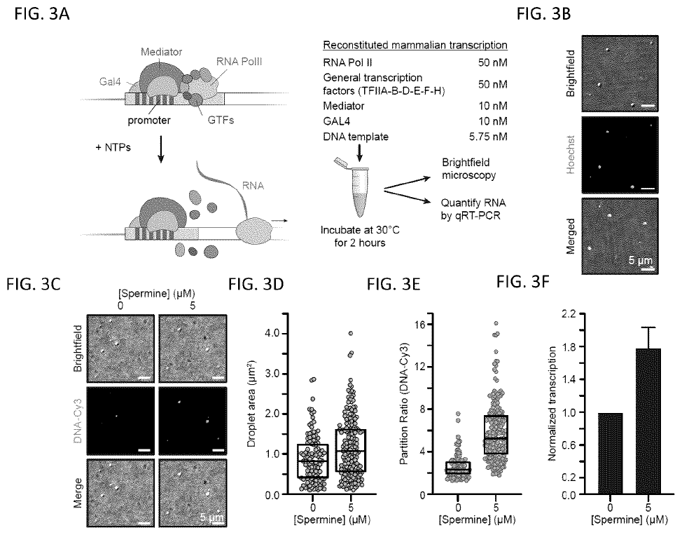

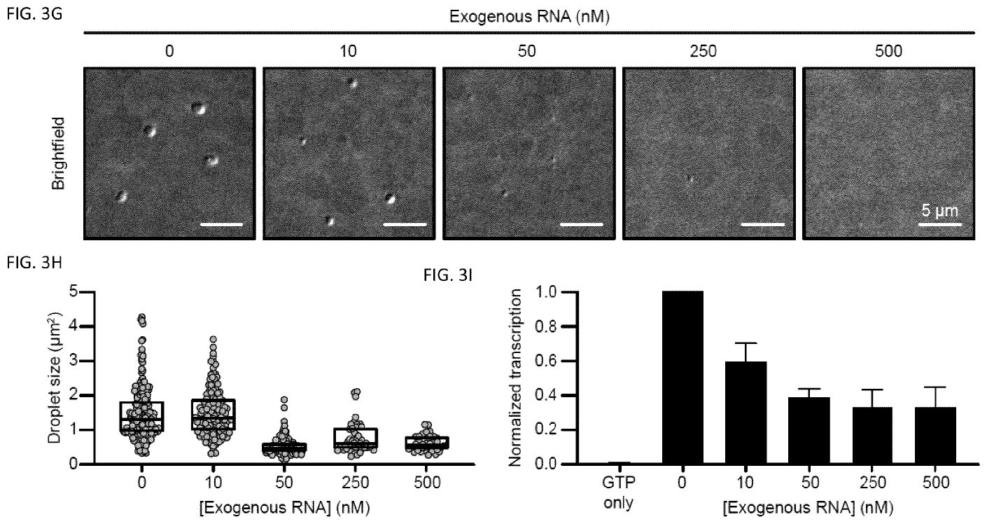

The inventors sought to investigate the functional consequence of the RNA-mediated reentrant phase behavior on transcription. Pol II-dependent transcription can be reconstituted in vitro with purified components (Roeder, 2019), so the inventors investigated whether droplets containing transcriptional components are formed in these assays and if conditions that alter droplet levels similarly alter transcriptional output. The inventors used a classical reconstituted mammalian transcription system with purified components, including Pol II, general transcription factors, Mediator and a transcriptional activator (Gal4), where addition of nucleotides permits transcription of a linear DNA template (FIG. 3A). The inventors observed that component mixtures and buffer conditions that are optimal for transcriptional output (Carey et al., 2009; Flores et al., 1992; LeRoy et al., 2008; Orphanides et al., 1998) produced droplets containing the DNA template (FIG. 3B). Quantification of the newly synthesized RNA in this system showed that 3.5 (±0.5) pM RNA was produced in the transcription reaction (STAR Methods). The inventors were unable to demonstrate that RNA synthesis actually occurs within the droplets because we cannot eliminate the possibility that synthesis occurs in the bulk phase and the product subsequently partitions into the droplet, but the observation that protein and template DNA concentrate in droplets under conditions optimal for transcription (FIG. 3B), and evidence that diverse condensate-altering treatments have similar effects on transcription, described below, is consistent with the notion that transcription occurs within condensates in this reconstituted system.

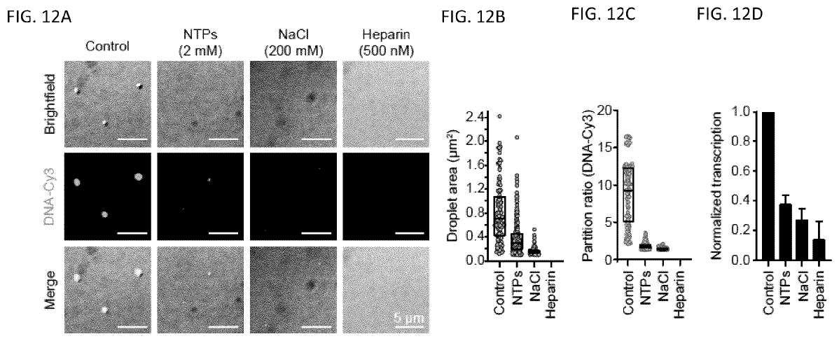

The inventors reasoned that if transcription and droplet formation are mutually dependent in the reconstituted system, then treatments that alter transcription should similarly impact condensate formation and vice versa. The addition to the reaction of various chemicals that are known to inhibit transcription (elevated concentrations of NTPs, NaCl, or heparin) (Carey et al., 2009; Reinberg and Roeder, 1987), caused reductions in droplet area, DNA partitioning and transcription (FIG. 12). Spermine, a positively-charged polyamine, will enhance droplet formation when it contributes to charge balance in coacervate models (Aumiller et al., 2016). Addition of spermine at concentrations predicted to balance charge in the in vitro reactions simultaneously increased droplet area, partitioning of template DNA, and levels of RNA synthesis (FIGS. 3C-3F, Table S2) (Blair, 1985; Moruzzi et al., 1975). These correlations suggest that optimal droplet formation and transcription are co-dependent.

An expectation of the RNA-feedback model is that droplets in the reconstituted system might ultimately produce enough RNA to cause a reduction in droplet size and transcriptional output. However, the low concentrations of RNA produced in these systems (3.5±0.5 pM, STAR Methods) are insufficient to dissolve the droplets. For this reason, the inventors tested whether purified RNA, added to the reaction, would similarly impact droplets and transcription. Indeed, addition of exogenous RNA reduced the number and size of the droplets (FIGS. 3G and 3H) and reduced template-derived RNA synthesis as measured by qRT-PCR (FIG. 3I). While these results do not rule out additional ways in which RNA may affect transcription (Pai et al., 2014), they are consistent with the expected behavior of transcriptional condensates if RNA contributes to negative feedback control.

### A Model for RNA-Mediated Non-Equilibrium Feedback Control of Transcriptional Condensates

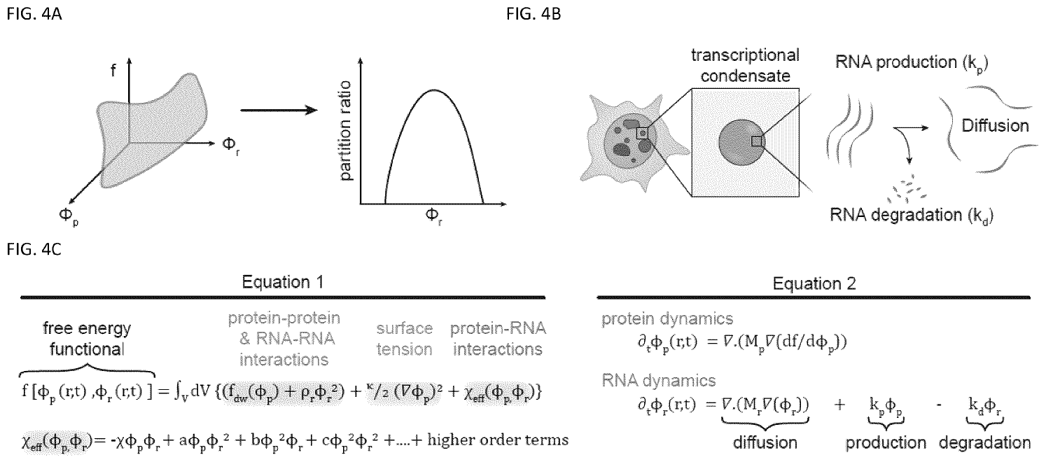

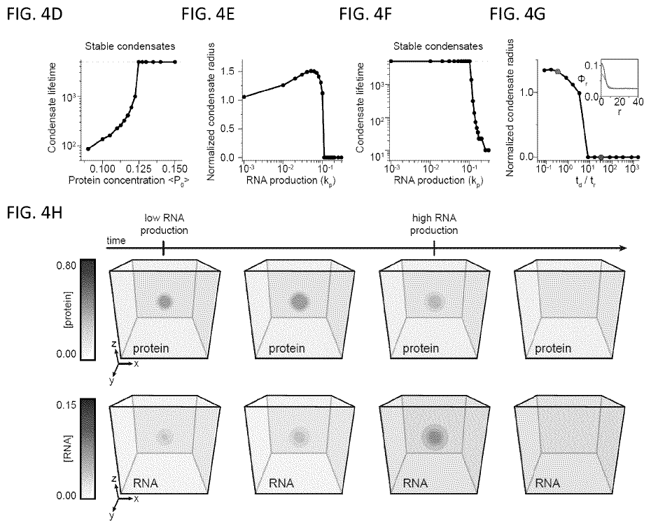

The in vitro experiments, which provide evidence that key transcriptional proteins and RNA exhibit an electrostatics-driven, RNA-protein ratio dependent, reentrant phase transition, were performed under equilibrium conditions (FIGS. 1 and 2). However, in vivo, RNA is synthesized and degraded at specific genomic loci by dynamic, ATP-dependent, non-equilibrium processes (Azofeifa et al., 2018; Li et al., 2016; Pefanis et al., 2015). To investigate how non-equilibrium processes underlying transcription may regulate transcriptional condensates, the inventors built a physics-based model with the goal of gaining mechanistic insights that could be tested experimentally. The model consists of two inter-linked parts: (1) A free-energy function (FIG. 4A), which depends on the concentrations of transcriptional proteins and RNA, that recapitulates the equilibrium reentrant phase behavior of RNA-protein mixtures (FIGS. 1 and 2). (2) A mathematical framework to study spatiotemporal evolution of condensates subject to dynamical processes of RNA synthesis, degradation, and diffusion (FIG. 4B).

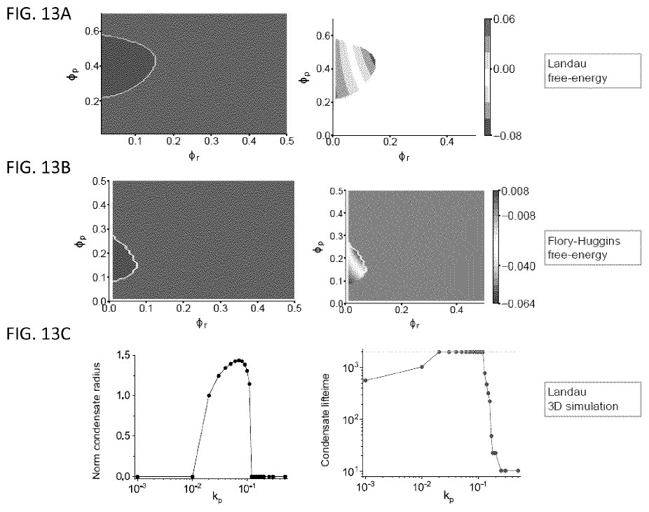

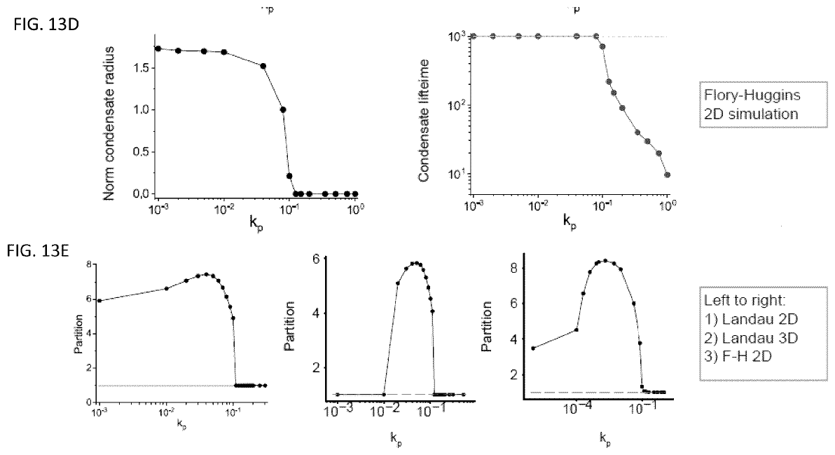

The inventors first developed a free-energy function to recapitulate the experimentally observed reentrant phase behavior of RNA-protein mixtures (FIG. 4A). The free energy function depends on the concentrations of transcriptional proteins and RNA (ϕₚ(\vec{r},t)) and (ϕᵣ(\vec{r}, t)), which vary in space and time. For simplicity, all transcriptional proteins are combined into one pseudo-species. Detailed quantitative models of RNA/protein condensates are analytically intractable (Adhikari et al., 2018; Delaney and Fredrickson, 2017; Zhang et al., 2018). Furthermore, our goal is not quantitative recapitulation of known experimental data, but rather to obtain mechanistic insights into RNA-mediated non-equilibrium regulation of transcription. Therefore, the inventors first sought to develop a free-energy function that qualitatively recapitulates the observed reentrant phase behavior of RNA/protein mixtures. Following a long tradition in the physics of phase transitions, the inventors employed a general Landau approach (Kardar, 2007; Landau, 1937) and expanded the free energy as a function of RNA and protein concentrations. The inventors included terms to describe repulsive RNA-RNA interactions, favorable interactions among the transcriptional proteins that drive condensate formation of transcriptional proteins in the absence of RNA (FIG. 4C, Eq. 1, in green), as well as a surface tension term important for describing condensate formation (FIG. 4C, Eq. 1 in blue, STAR Methods). The free energy function also includes protein-RNA interactions that are described by a concentration-dependent interaction term, which is expanded in the standard Landau fashion (FIG. 4C, Eq. 1, in red) (Kardar, 2007). Magnitudes of the coefficients of the various terms in the expansion (χ,a,b,c) account for the effective strength of RNA-protein interactions (STAR Methods), which implicitly include solvent effects. While symmetry arguments do not preclude any specific terms in this expansion, the choice of (χ>0, c>0, a, b<<1) ensures a reentrant phase transition (schematic in FIGS. 4B, 13A, STAR Methods) with a minimal number of higher order terms. The inventors established that this choice of coefficients leads to a reentrant transition by analyses of the Jacobian matrix (see STAR Methods). Results using the Landau model (FIG. 4C, Eq. 1) are recapitulated using a different method for obtaining the free-energy (Flory-Huggins) to highlight the generality of our Landau approach (FIGS. 13A and 13B, STAR Methods). Given the universality of its application, easily characterizable phase behavior, and numerical ease of investigation (e.g. ˜50 times faster than the Flory-Huggins to study coupled dynamics), the inventors employed the Landau free-energy for the rest of this example to study how the dynamics of transcriptional condensates is regulated by transcription.

The inventors next developed a mathematical framework to study the temporal evolution of transcriptional condensates as transcription ensues. Most transcriptional proteins turn-over with a half-life of several hours (Cambridge et al., 2011; Chen et al., 2016), which is longer than time-scales of transcription-associated events, which range from seconds to minutes (Chen and Larson, 2016; Fukaya et al., 2016; Rodriguez and Larson, 2020). Hence, the overall amount of protein is conserved in the timescales of interest. Thus, the dynamics of the protein concentration (ϕₚ) are represented by standard Model B dynamics (FIG. 4C, Eq. 2) (Hohenberg and Halperin, 1977). Under Model B dynamics, gradients in the protein chemical potential, which depend on both the spatial distribution of protein and RNA concentrations, drives diffusive protein fluxes, which in turn drives the spatio-temporal evolution of ϕₚ. Since RNA concentrations vary over transcription-associated time-scales, the dynamics of ϕᵣ is explicitly governed by a reaction-diffusion equation. The key features (schematic in FIG. 4B) are that RNA diffuses with mobility Mᵣₙₐ and is synthesized and degraded with specific reaction rates, kₚ and k_{d}, respectively. Because the RNA dynamics are far from equilibrium and the free energy function noted above depends upon both protein and RNA concentrations, the coupled temporal evolution of transcriptional proteins and RNA (FIG. 4C, Eqs. 1 and 2) cannot be obtained from near-equilibrium considerations of simply going downhill in free energy with time. The inventors employ this mathematical framework to study non-equilibrium regulation of transcriptional condensates.

The inventors first sought to determine whether this model is consistent with previous studies (Cho et al., 2016, 2018). These studies have shown that transcriptional condensates at different genomic loci recruit a varying number of transcriptional proteins, which in turn, correlates with condensate lifetimes. To explore this phenomenon, the inventors numerically simulated Eq. 2 (FIG. 4C) on 2- and 3-dimensional grids (STAR methods). Locus-dependent recruitment of the transcriptional machinery can be mimicked in this model by varying the total transcriptional protein amount ( [symbol] P₀ [symbol] ) with all other parameters fixed, as this simulation volume represents a local micro-environment (FIG. 4A). The inventors simulations predict that loci that can recruit more transcriptional proteins (higher [symbol] P₀ [symbol] ) form relatively stable condensates, while condensates that recruit fewer proteins dissolve after a characteristic lifetime (FIG. 4D). The model predictions for transcriptional condensate dynamics are qualitatively consistent with published data (Cho et al., 2016), and suggest that features encoded at genomic loci contribute to transcriptional condensate dynamics.

The inventors next investigated how the sizes and lifetimes of transcriptional condensates change as a function of the effective rate of RNA synthesis (kₚ), while keeping all other parameters fixed. In these simulations, the size of condensates initially increases and subsequently decreases with increasing effective rates of RNA synthesis (FIG. 4E). Above a threshold rate of RNA synthesis, condensates dissolve (FIG. 4E). The underlying reason for this result is the reentrant phase behavior of mixtures of transcriptional molecules and RNA (FIGS. 1 and 2). The inventors also find that condensates with higher transcriptional activity dissolve faster, as measured by condensate lifetimes (FIG. 4F). Condensate lifetimes do not vary over a range of RNA transcription rates that reflect RNA-transcriptional protein ratios that roughly correspond to the charge balance conditions (FIG. 4F). The same qualitative results are recapitulated in 3D simulations (FIG. 13C) as well as simulations employing the Flory-Huggins free-energy (FIG. 13D), and further reinforced by partition ratios computed from simulations (FIG. 13E). Overall, these results suggest a model wherein low effective rates of RNA synthesis (or low transcription activity) stabilize transcriptional condensates while higher rates promote condensate dissolution.

The inventors then investigated the extent to which non-equilibrium effects underlying transcription regulate transcriptional condensate dynamics. RNA synthesis, degradation, and diffusion influence the spatial distribution of RNA, which in turn, may feedback on transcriptional condensates. To explore this, the inventors varied the diffusivity of RNA and the effective rates of RNA synthesis and degradation, while holding the ratio of synthesis and degradation rates constant. The latter constraint ensures that the overall RNA concentration is constant in the condensate as other parameters are varied, thus any effect on condensate dynamics arises from purely non-equilibrium effects. Varying the parameters that control RNA synthesis/degradation rates and diffusion changes the relative time-scales of these processes (tᵣ and t_{d}, respectively) (STAR Methods), which in turn, influences the spatial distribution of RNA in the condensate. If diffusion is slower than synthesis/degradation (tᵣ<t_{d}), then RNA will accumulate near transcription sites, leading to a higher local RNA concentration in the condensate. Conversely, if diffusion is faster than synthesis/degradation (tᵣ>t_{d}), then RNA will diffuse away from transcription sites, leading to a lower uniform RNA concentration in the condensate. The spatial distribution of RNA will impact condensates according to local charge balance. To study how varying spatial distributions of RNA affect transcriptional condensates, the inventors simulated conditions where the overall RNA concentration was fixed close to the charge-balance condition, thus promoting condensate formation at equilibrium. In these simulations, condensates that are stable when synthesis/degradation is slower than diffusion (tᵣ>t_{d}) dissolve when RNA synthesis/degradation is faster than diffusion (tᵣ<t_{d}) (FIG. 4G). When (tᵣ>t_{d}), RNA concentration is relatively uniform and low throughout the condensate and equilibrium effects dominate. Conversely, when (tᵣ<t_{d}), RNA is distributed non-uniformly with high local concentrations in the condensate and non-equilibrium effects dominate to result in condensate dissolution (FIG. 4G). In the latter case, the localized high RNA concentrations exceed the charge balance condition due to non-equilibrium effects. Approximate estimates for the rates of RNA synthesis, degradation, and diffusion under physiological conditions (t_{d}/tᵣ≈2-100, STAR Methods) suggest that transcriptional condensate dynamics are likely driven off equilibrium.

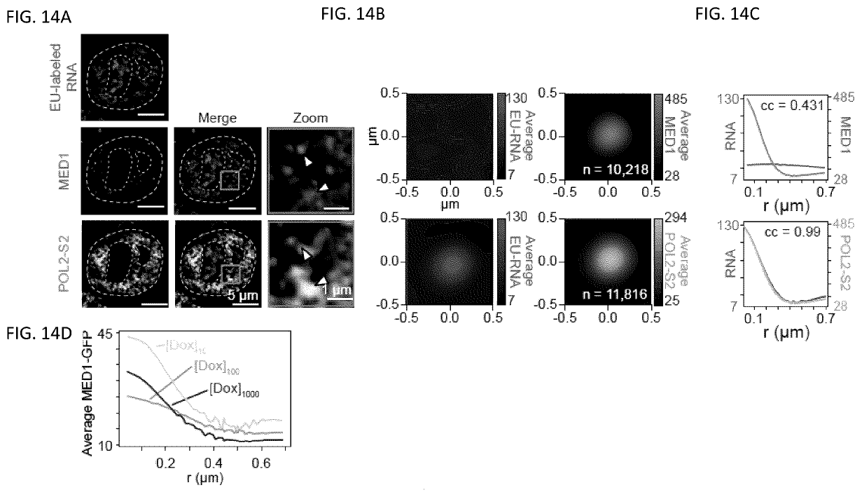

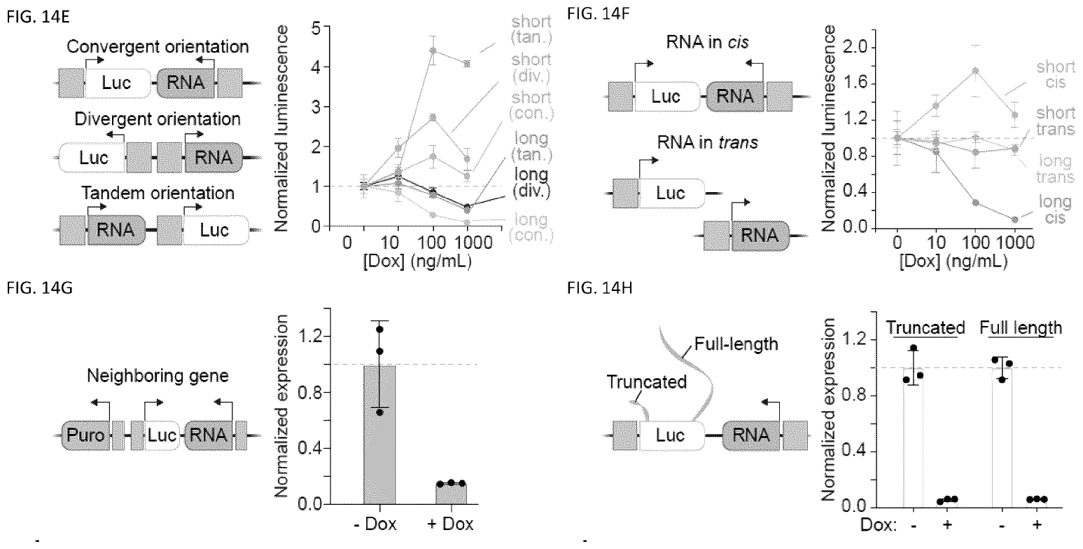

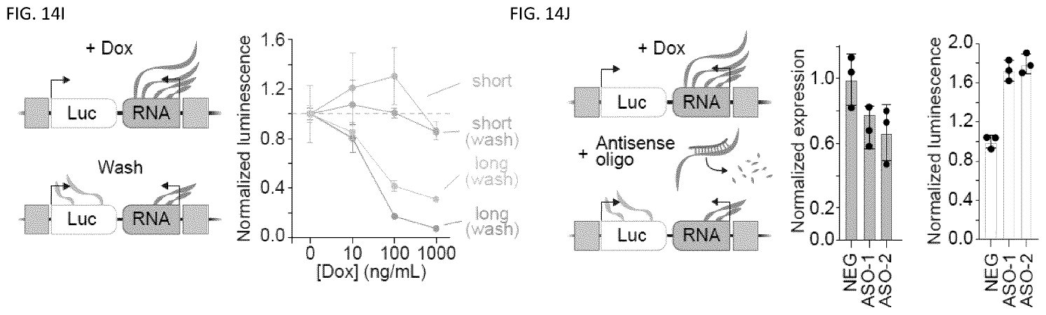

The inventors sought to synthesize our results so far to explore the effect of non-equilibrium dynamics on regulating transcriptional condensates across transcription initiation and productive elongation. Simulations were started at a relatively low effective rate of RNA synthesis, mimicking initiation, followed by an increase to a relatively high effective rate of RNA synthesis, mimicking productive elongation. The simulations predict that low effective rates of RNA synthesis enhance condensate formation, and these condensates subsequently dissolve upon ensuing higher effective rates of RNA synthesis (FIG. 4H). Consistent with these simulations, Mediator condensates tend to be depleted in areas of high, Pol II-driven nascent transcription (FIG. 14A-14C). These results suggest that non-equilibrium processes underlying RNA synthesis can potentially regulate the formation and dissolution of transcriptional condensates.

### Inhibition of RNA Elongation Leads to Enhanced Condensate Size and Lifetime in Cells

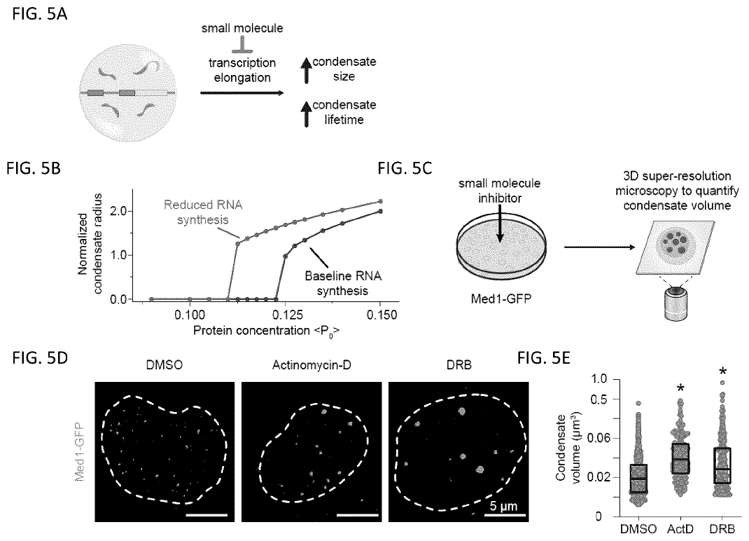

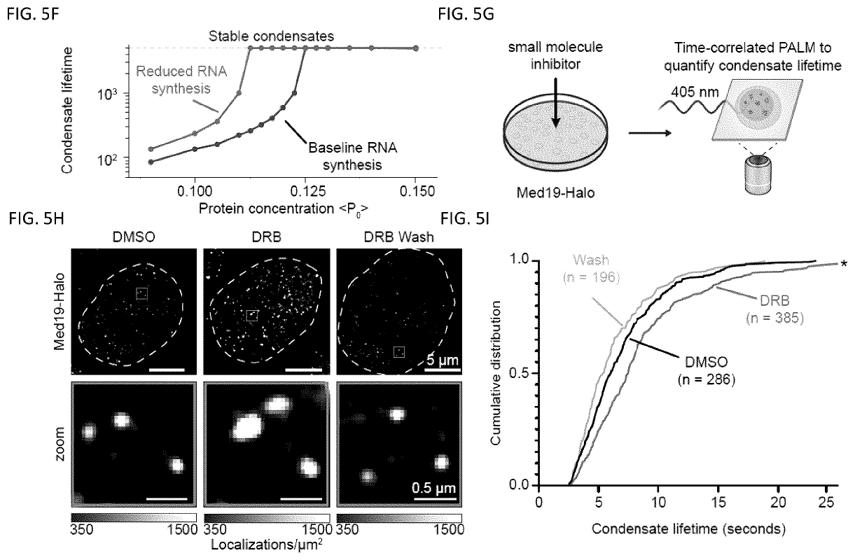

Transcriptional condensates in cells are highly dynamic, forming and dissolving at timescales ranging from seconds to minutes (Cho et al., 2018). The inventors previously showed that condensate formation is associated with transcription activation and initiation (Cho et al., 2018). Once transcriptional condensates are formed, the RNA-mediated condensate dissolution model predicts that inhibition of elongation should increase the size and lifetime of transcriptional condensates (FIG. 5A). The inventors used the physics-based model (FIGS. 4A-4C) to simulate the effects of elongation inhibition on transcriptional condensates and performed experiments to test the predictions from these simulations in cells (FIGS. 5B-51). In order to account for the locus-dependent ability to recruit the transcriptional machinery and Pol II, the inventors performed these simulations at a range of total protein concentrations (as in FIGS. 4D and 4E), but for conditions where the effective rate of RNA synthesis (kₚ) was high (corresponding to elongation) and low (corresponding to inhibited elongation). The results of the simulations predict that a reduced effective rate of RNA synthesis should increase the size and lifetime of transcriptional condensates across a range of total protein concentrations (FIGS. 5B and 5F).

To experimentally test these predictions from the simulations, mESCs engineered with an endogenous, GFP-tagged subunit of Mediator (Med1-GFP) (Sabari et al., 2018) were treated for 30 minutes with Actinomycin-D or DRB (FIG. 5C), which disrupt transcription elongation through DNA intercalation and inhibition of CDK9-mediated Pol II pause release, respectively (Singh and Padgett, 2009; Sobell, 1985; Steurer et al., 2018). Consistent with the model predictions, after inhibition of elongation, Med1-GFP condensates increased in volume by ˜2-fold as measured by 3D super-resolution microscopy (FIGS. 5D and 5E). Condensate lifetime could not be assessed in these cells due to the long duration of image acquisition and consequent photobleaching, so the inventors turned to time-correlated PALM super-resolution microscopy (tcPALM) in mESCs with an endogenous Med19-Halo tag (Cho et al., 2018; Cisse et al., 2013) to investigate the effects of elongation inhibition on condensate lifetime (FIG. 5G). Cells were treated for 30 minutes with DRB to disrupt transcription elongation, and the lifetime of Med19 condensates was quantified. When transcription elongation was inhibited by DRB treatment, Med19 condensates exhibited significantly longer lifetimes than mock-treated cells (FIGS. 5H and 51), and when DRB-treated cells were washed with fresh media, the lifetimes of the Med19 condensates recovered to those of the mock-treated condition (FIGS. 5H and 51). Taken together, the in silico and experimental results show that suppression of elongation in cells leads to increased condensate size and lifetime, consistent with the model that a burst of RNA synthesis can promote dissolution of transcriptional condensates in cells.

### Increasing the Levels of Local RNA Synthesis Reduces Condensate Formation and Transcription in Cells

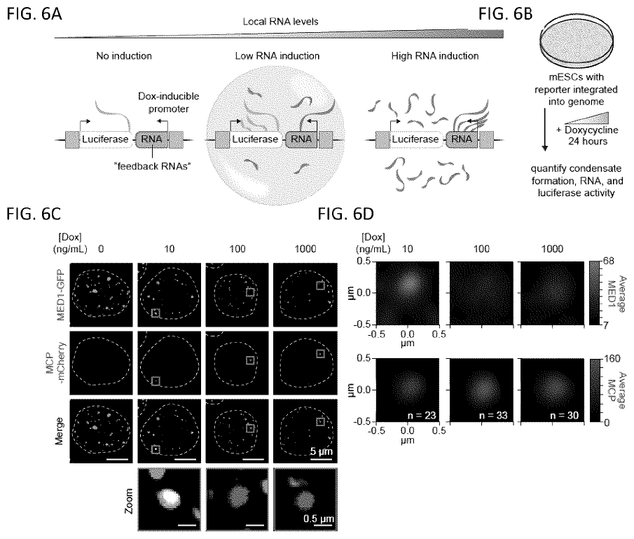

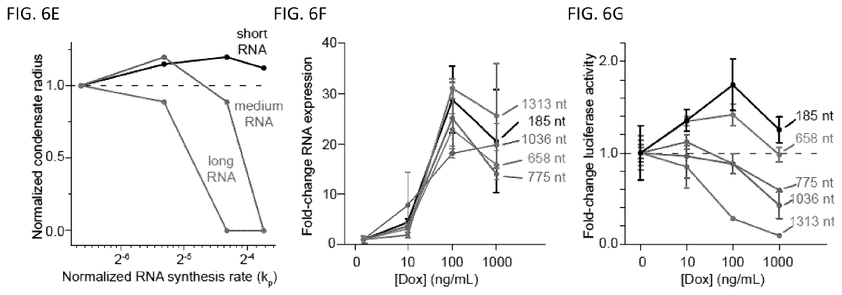

The RNA-mediated feedback model suggests that modifying the concentration or size of RNA molecules should have a predictable effect on transcriptional output. The inventors developed complementary experimental and simulation approaches (FIG. 6) whereby the levels of putative “feedback RNAs” could artificially be increased. The inventors first used the physics-based model (FIG. 4) to simulate the effect of increasing effective rates of RNA synthesis as well as varying lengths for the synthesized RNA on condensates (STAR Methods). The simulations predicted that increases in the production rate of shorter RNAs initially enhance and subsequently suppress transcriptional condensate size, while increases in the production rate of longer RNAs lead to reduced condensate size with increasing synthesis rates (FIG. 6E).

To test this prediction, the inventors investigated the effect of artificially increasing the levels of feedback RNAs on the transcription of an adjacent luciferase reporter gene in cells (FIGS. 6A and 6B) (Kirk et al., 2018). DNA molecules specifying RNAs of a range of sizes were cloned into this system to allow dox-inducible expression of these RNAs, and mESC lines were generated with clones of integrated constructs. Feedback RNAs were observed at loci of Mediator puncta under low dox stimulation, suggesting that these actively transcribed genes are associated with transcriptional condensates (FIGS. 6C, 6D). Elevated expression of feedback RNAs under higher dox stimulation reduced their colocalization with Mediator puncta, consistent with the model of RNA-mediated feedback on condensates (FIGS. 6C, 6D, and 14D). To study the effect of local RNA levels on transcription, additional cell lines harboring diverse feedback RNAs were then treated with increasing doses of doxycycline to induce feedback RNA expression (FIG. 6F) and reporter expression was measured by luminescence (FIG. 6G). The results were consistent with model predictions (FIG. 6E): increases in the levels of short feedback RNAs initially enhanced reporter expression and then suppressed this, while progressive increases in the levels of the longer feedback RNAs more strongly reduced reporter expression (FIG. 6G). The inventors confirmed that changes in reporter expression arise from cis RNA-mediated effects by modifying the constructs, controlling for the global effects of Dox, and perturbing local RNA concentration (FIGS. 14E-14J). Together, these results support a role for RNA-mediated feedback control of transcriptional condensates.

### Discussion

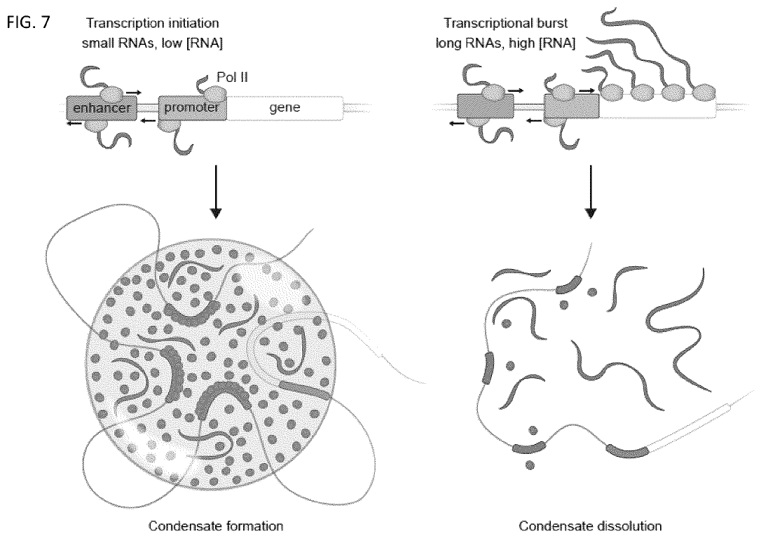

The results described here indicate that transcription is a non-equilibrium process that provides dynamic feedback through its RNA product. The results support a model whereby RNA provides both positive and negative feedback on transcription via the regulation of electrostatic interactions in transcriptional condensates. Transcriptional condensates, whose production involves crowding of transcription factors by enhancer DNA (Shrinivas et al., 2019) and electrostatic and other interactions between the IDRs of transcription factors and coactivators (Boija et al., 2018; Sabari et al., 2018), engage RNA to both promote and dissolve the condensates. In this RNA feedback model, low levels of short RNAs produced during transcription initiation promote formation of transcriptional condensates, while high levels of the longer RNAs produced during elongation can cause condensate dissolution (FIG. 7).

RNA-mediated feedback regulation of transcription is the result of coupling between the non-equilibrium processes of RNA synthesis, degradation, and diffusion with an underlying equilibrium phase behavior of RNA-transcriptional protein mixtures that exhibit a reentrant phase transition. Such phase transitions have been observed in prior studies of complex coacervation in mixtures of oppositely charged polyelectrolyte solutions, including RNA-polyelectrolyte mixtures (Lin et al., 2019; Overbeek and Voorn, 1957; Sing, 2017; Srivastava and Tirrell, 2016). The inventors provided several lines of evidence to show that mixtures of RNA and transcriptional molecules undergo a reentrant phase transition at equilibrium. In droplet formation assays, low levels of RNA that occur at gene regulatory regions can stimulate condensate formation by the Mediator coactivator whereas high levels suppress condensates (FIG. 1). When these experiments were repeated with the MED1-IDR, a disordered component of Mediator that contributes to transcriptional condensates, the effects of varying RNA molecules on droplets fit expectations for the charge balance model (FIG. 2).

Using a physics-based model, the inventors then studied how non-equilibrium processes of RNA synthesis, degradation, and diffusion are linked to equilibrium reentrant phase behavior to regulate the size and dynamics of transcriptional condensates in vivo. In agreement with predictions from the model, the dependence of these quantities and transcriptional output on RNA synthesis rates and lengths were then positively tested in cell-free and in cellular systems (FIGS. 4-6). Together, these results suggest that the coupling of non-equilibrium processes inherent to RNA transcription with a phenomenon akin to complex coacervation plays an important role in regulating transcriptional condensates and their output in vivo. Previous theoretical efforts have explored how non-equilibrium processes may be coupled to condensate formation (Weber et al., 2019; Zwicker et al., 2017). This study provides a framework to understand how non-equilibrium regulation of condensates is important for a specific biological process, namely transcription.

An RNA-mediated feedback model for transcriptional regulation provides a potential explanation for the roles of enhancer and promoter-associated RNAs, which are evolutionarily conserved features of eukaryotes. These low-abundance short RNAs, transcribed bidirectionally from enhancers and promoters, have been reported to affect transcription from their associated genes through diverse postulated mechanisms (Andersson et al., 2014; Catarino and Stark, 2018; Core et al., 2014; Gardini and Shiekhattar, 2015; Henriques et al., 2018; Lai et al., 2013; Li et al., 2016; Mikhaylichenko et al., 2018; Nair et al., 2019; Pefanis et al., 2015; Rahnamoun et al., 2018; Schaukowitch et al., 2014; Scruggs et al., 2015; Sigova et al., 2015; Smith et al., 2019). The diversity of sequences present in these short RNA species has made it difficult to postulate a common molecular mechanism for their effects on transcription. In this context, a model for RNA-mediated feedback regulation of condensates is attractive for several reasons. RNA molecules are known components of other biomolecular condensates, including the nucleolus, nuclear speckles, paraspeckles and stress granules, where they are known to play regulatory roles (Fay and Anderson, 2018; Roden and Gladfelter, 2020). RNA is a powerful regulator of condensates that are formed by electrostatic forces because it has a high negative charge density due to its phosphate backbone (Drobot et al., 2018; Frankel et al., 2016), thus explaining why the effects of diverse RNAs on transcriptional condensates are sequence-independent.

Recent studies indicate that transcription occurs in periodic bursts (˜1-10 minutes in duration), where multiple molecules of Pol II can be released from promoters within a short timeframe and produce multiple molecules of mRNA (˜1-100 molecules per burst) (Cisse et al., 2013; Fukaya et al., 2016; Larsson et al., 2019). Multiple models explain such periodic bursts through stochastic gene activation events (Chen and Larson, 2016; Larsson et al., 2019; Raj et al., 2006; Rodriguez and Larson, 2020; Suter et al., 2011; Tunnacliffe and Chubb, 2020) but are often agnostic to the underlying mechanism or attribute these to rate-limiting transcription factor binding events. The inventors suggest that a rapid and spatially-localized change in charge balance, due to increased RNA synthesis at pause release of active Pol II, may contribute to dissolution of transcriptional condensates and thus dynamic loss of the pool of transcriptional apparatus in those condensates. This would provide a means to provide negative feedback to arrest transcription and a mechanism that may contribute to the dynamic bursty behavior observed for transcription.

### Star Methods

### Data/Code Availability Statement

The code generated during this study is available on the worldwide web at

github.com/krishna-shrinivas/2020_Henninger_Oksuz_Shrinivas_RNA_feedback.

### Cell Culture

The Jaenisch laboratory gifted the V6.5 mouse ES cells. ES cells were maintained at 37° C. with 5% CO2 in a humidified incubator on 0.2% gelatinized (Sigma, G1890) tissue-culture plates in 2i medium with LIF, which was made according to the following recipe: 960 mL DMEM/F12 (Life Technologies, 11320082), 5 mL N2 supplement (Life Technologies, 17502048; stock 100×), 10 mL B27 supplement (Life Technologies, 17504044; stock 50×), 5 mL additional L-glutamine (Gibco 25030-081; stock 200 mM), 10 mL MEM nonessential amino acids (Gibco 11140076; stock 100×), 10 mL penicillin-streptomycin (Life Technologies, 15140163; stock 10\hat{ }4 U/mL), 333 μL BSA fraction V (Gibco 15260037; stock 7.50%), 7 μL β-mercaptoethanol (Sigma M6250; stock 14.3 M), 100 μL LIF (Chemico, ESG1107; stock 10\hat{ }7 U/mL), 100 μL PD0325901 (Stemgent, 04-0006-10; stock 10 mM), and 300 μL CHIR99021 (Stemgent, 04-0004-10; stock 10 mM). For confocal and PALM imaging, cells were grown on glass coverslips (Carolina Biological Supply, 633029) that had been coated with the following: 5 μg/mL of poly-L-ornithine (Sigma P4957) at 37° C. for at least 30 minutes followed by 5 μg/mL of laminin (Corning, 354232) at 37° C. for at least 2 hours. Cells were passaged by washing once with 1×PBS (Life Technologies, AM9625) and incubating with TrypLE (Life Technologies, 12604021) for 3-5 minutes, then quenched with serum-containing media made by the following recipe: 500 mL DMEM KO (Gibco 10829-018), MEM nonessential amino acids (Gibco 11140076; stock 100×), penicillin-streptomycin (Life Technologies, 15140163; stock 10\hat{ }4 U/mL), 5 mL L-glutamine (Gibco 25030-081; stock 100×), 4 μL β-mercaptoethanol (Sigma M6250; stock 14.3 M), 50 μL LIF (Chemico, ESG1107; stock 10\hat{ }7 U/mL), and 75 mL of fetal bovine serum (Sigma, F4135). Cells were passaged every 2 days.

### ChIP-Seq Analysis

ChIP-seq browser tracks for MED1, Pol II, BRD4, and OCT4 were generated as described (Sabari et al., 2018; Whyte et al., 2013). Briefly, reads were aligned to NCBI37/mm9 using Bowtie with the following settings: “-p 4 --best-k 1-m 1 --sam-1 40”. WIG files represent counts (in reads per million, floored at 0.1) of aligned reads within 50 bp bins. Each read was extended by 200 nt in the direction of the alignment.

(Source: www.ncbi.nlm.nih.gov/geo/query/acc.cgi?acc=GSE112808)

### GRO-Seq Analysis

For generation of the GRO-seq browser tracks, GRO-seq reads were processed as described in (Sigova et al., 2015). The GRO-seq .sra file corresponding to GEO accession number GSM1665566 (Sigova et al., 2015) was converted to .fastq using the SRA toolkit (Leinonen et al., 2011). Reads were aligned to the mouse genome (NCBI37/mm9) using Bowtie v1.2.2 (Langmead et al., 2009) with the following settings “-e 70-k 1-m 10-n 2 --best”. The reads corresponding to each one of the features (super-enhancers, typical enhancers, proximal promoter regions, genes) were counted using featureCounts v1.6.2 (Liao et al., 2014) with default settings. The coordinates for typical enhancers and super-enhancers in mouse embryonic stem cells (mESCs) were acquired from (Whyte et al., 2013). The coordinates for genes (transcription start and end sites) were acquired using the UCSC Table Browser (Karolchik et al., 2004). The upstream antisense promoter regions were defined as genomic areas containing 1 kb upstream of each TSS. Their coordinates were retrieved by using BEDTools v.2.26.0 (Quinlan and Hall, 2010) and the TSS coordinates as input (to the slop function). Reads were normalized with the size of the corresponding feature they aligned to.

### RNA-Seq Analysis

The RNA-seq .sra file corresponding to GEO accession number GSM2686137b (Chiu et al., 2018) was converted to .fastq using the SRA Toolkit RNA-seq analysis was performed using the nf-core RNA-seq pipeline (v1.4.2) (Ewels et al., 2020) with default settings and NCBI37/mm9 as reference genome. Nextflow v20.01.0 was used as a workflow tool on an LSF High-Performance Computing environment (Di Tommaso et al., 2017). STAR v2.6.1d (Dobin et al., 2013) was used for the alignment of reads. Aligned reads were assigned to the aforementioned intervals (typical enhancers, super-enhancers, proximal promoter regions and genes) by using featureCounts v1.6.4, with the default settings.

### Calculation Number of RNA Molecules in Cells

Known concentrations of in vitro transcribed enhancer RNAs and pre-mRNAs from Trim28 and Pou5f1 loci are used as standards to approximate the number of molecules in cells. These RNAs are converted to cDNAs by reverse-transcription and mixed at equal concentrations. For each RNA species, a standard curve of qRT-PCR Ct value to RNA amount was generated using serial dilutions, with two different primer sets in technical duplicates. Next, qRT-PCR reactions using the same primer sets were performed for biological duplicates of mESCs. Actb-normalized Ct values were then used to determine the amount of RNA species in the reaction based on the standard curves above. To calculate the number of RNA molecules per cell, the amount of RNA (g) was divided by the molar weight of each species (˜350 (g mol⁻¹ nt⁻¹)×length of in vitro transcribed RNA (nt)), multiplied by Avogadro's number (6.022×10²³ mol⁻¹), and divided by the approximate number of cells used in each reaction (10,000 cells). Melting curves were analyzed to confirm primers specificity. Non-reverse-transcribed (−RT) controls were included to rule out the amplification of genomic DNA. Primer sequences are indicated in Table S1.

### In Vitro Droplet Assay

Recombinant GFP fusion proteins were concentrated to a desired protein concentration using Amicon Ultra centrifugal filters (30K MWCO, Millipore). Droplet reactions with the recombinant proteins were performed in 10 ul volumes in PCR tubes under the following buffer condition: 30 mM Tris HCl PH 7.4, 100 mM NaCl, 2% Glycerol and 1 mM DTT. The same buffer containing 55 mM NaCl was used for BRD4-IDR-GFP. Droplet reactions with the Mediator complex were performed under the following buffer condition: 30 mM HEPES pH 7.4, 65 mM NaCl, 2% Glycerol and 1 mM DTT. For all droplet reactions, protein and buffer were mixed first and RNA or ssDNA or heparin (Sigma, H3393) was added later. The reactions were incubated at room temperature for 1 hr without any shaking or rotating. The reactions were then individually transferred into 384 well-plate (Cellvis P384-1.5H-N) by using a micropipette (2-20 μL) 5 minutes prior to imaging by confocal microscopy at 150× magnification or prior to turbidity measurements on a plate reader (Tecan) at 350 nm absorbance at room temperature (Banerjee et al., 2017). The concentration of proteins and RNAs in the droplet reactions are indicated in the figure legends. For brightfield Mediator experiments (FIG. 1), representative images were subtracted by a median filtered image (px=15) using ImageJ to remove camera artifacts discovered by taking images of blank wells.

Fluorescence Recovery after Photobleaching (FRAP)

FRAP was performed on an Andor Revolution Spinning Disk Confocal microscope with 488-nm laser. Droplets were bleached using 30% laser power with 20 us dwell time for 5 pulses, and images were collected every second for 60 seconds. Fluorescence intensity at the bleached spot and a control unbleached spot was measured using ImageJ. Values are normalized to the unbleached spot to control for photobleaching during image acquisition and then normalized to the first time point intensity.

MATLAB™ scripts were written to process the intensity data, and post bleach FRAP recovery data was normalized to pre-bleach intensity (FRAP(t)) and fit to:

$$FRAP ( t ) = M ( 1 - exp ( - t / τ ) )$$

Where, M (mobile fraction) and t (half-life of recovery) are inferred in-built MATLAB functions. These values are inferred for each replicate and averaged to provide a range for the apparent diffusion coefficients, which is computed as:

$${D}_{app} = {( Bleach radius )}^{2} / τ$$

### In Vitro Droplet Analysis

To analyse in vitro droplet experiments, the inventors used a previously reported pipeline (Guo et al., 2019). The code for this analysis is available at the Github link in the Data/Code Availability section. Briefly, all droplets were segmented from average images of captured channels on various criteria: (1) an intensity threshold that was three s.d. above the mean of the image; (2) size thresholds (20 pixel minimum droplet size); and (3) a minimum circularity (circularity=4π·(area)/(perimeter²)) of 0.8 (1 being a perfect circle). After segmentation, mean intensity for each droplet was calculated while excluding pixels near the phase interface. Hundreds of droplets identified in (typically) ten independent fields of view were quantified. The mean intensity within the droplets (C-in) and in the bulk (C-out) were calculated for each channel. The partition ratio was computed as (C-in)/(C-out).

Droplet size, partition ratio, and condensed fraction measure distinct properties of droplet formation, and these three metrics show similar trends upon RNA-mediated reentrant phase transitions. When a protein or RNA is fluorescently-labeled in our experiments, we favor measuring the partition ratio. This is because the partition ratio can be measured on a per-droplet basis, and unlike condensed fraction, which varies depending on the number of droplets per field, the partition ratio is more independent of the field that is imaged.

For the size analysis of droplets formed in the reconstituted transcription assays (FIG. 3), brightfield images were subtracted by a median-filtered image (px=21), and droplets were manually segmented and their areas measured using ImageJ.

### Synthesis of RNA by In Vitro Transcription

Enhancer and promoter sequences for RNAs were obtained from super-enhancer-regulated genes Pou5f1, Nanog, and Trim28. For promoter sequences, the first 475-490 bp from the first exon were selected from mm10. For enhancer sequences, GROseq reads (Sigova et al., 2015) from both + and − strands aligned to mm9 were overlapped with called super-enhancers (Whyte et al., 2013). Contiguous regions of read density above background were manually selected (FIGS. 8A-8F). Primers were designed to amplify the selected promoter and enhancer sequences from genomic DNA isolated from V6.5 mESCs (Table S1). The following sequences were added to the forward and reverse primers to add the bacterial polymerase promoters:

| | T7 (add to 5′ of sense or |
|---|---|
| | forward primer): |
| (SEQ ID NO: 1) | |
| | 5′-TAATACGACTCACTATAGGG-3′ |
| | SP6 (add to 5′ of antisense |
| | or reverse primer): |
| (SEQ ID NO: 2) | |
| | 5′-ATTTAGGTGACACTATAGAA-3′ |

Phusion polymerase (NEB) is used to amplify the products with the bacterial promoters, and products are run on a 1% agarose gel, gel-purified using the Qiaquick Gel Extraction Kit (Qiagen), and eluted in 40 μL H2O. Templates were sequenced to verify their identity. A volume of 8 μL of each template (10-40 ng/μL) was transcribed using the MEGAscript T7 (Invitrogen; sense) or MEGAscript SP6 (Invitrogen; antisense) kits according to the manufacturer's instructions. For visualization of the RNA by microscopy, reactions included a Cy5-labeled UTP (Enzo LifeSciences ENZ-42506) at a ratio of 1:10 labeled UTP:unlabeled UTP. The in vitro transcription was incubated overnight at 37° C., then 1 μL TURBO DNAse (supplied in kit) was added, and the reaction was incubated for 15 minutes at 37° C. The MEGAclear Transcription Clean-Up Kit (Invitrogen) was used to purify the RNA following the manufacturer's instructions and eluting in 40 μL H2O. RNA was diluted to 2 μM and aliquoted to limit freeze/thaw cycles, and RNA was run on 1% agarose gels in TBE buffer to verify a single band of correct size.

### Recombinant Protein Purification

Recombinant protein purifications were performed as previously reported (Boija et al., 2018; Guo et al., 2019; Sabari et al., 2018; Shrinivas et al., 2019; Zamudio et al., 2019). Briefly, pET expression plasmids containing 6×HIS tag and genes of interest or their IDRs tagged with either mEGFP or mCherry were transformed into LOBSTR cells (gift of I. Cheeseman Lab). Expression of proteins was induced by addition of 1 mM IPTG either at 16° C. for 18 hours or at 37° C. for 5 hours. Extracts were prepared as previously described (Boija et al., 2018). Proteins were purified by Ni-NTA agarose beads (Invitrogen, R901-15), and eluted with 50 mM Tris pH 7.4, 500 mM NaCl, 250 mM imidazole buffer containing complete protease inhibitors (Roche, 11873580001). Proteins were dialyzed against 50 mM Tris pH 7.4, 125 mM NaCl, 10% glycerol and 1 mM DTT at 4° C. for BRD4-IDR-GFP, OCT4-GFP and GFP alone and the same buffer containing 500 mM NaCl for MED1-IDR-GFP.

Purification of Human Mediator Complex from HeLa Nuclear Extract.

HeLa nuclear protein extract (4 g) was prepared as described in (Dignam et al., 1983). Nuclear extract was dialyzed against BC100: BC buffer, pH 7.5+100 mM KCl (20 mM Tris-HCl, 20 mM B-Mercaptoethanol, 0.2 mM PMSF, 0.2 mM EDTA, 10% glycerol (v/v) and 100 mM KCl). The extract was fractionated on a phosphocellulose column (P11) with BC buffer containing 0.1, 0.3, 0.5 and 1M KCl. The Mediator complex eluted in the 0.5M KCL (BC500) fraction. This fraction was dialyzed against BC100 and loaded on a DEAE Cellulose column and sequentially fractionated with BC buffer containing 0.1, 0.3 and 0.5M KCl. The Mediator did not bind the DEAE Cellulose resin and was collected in the flow through fraction 0.1M KCl (BC100). This fraction was then directly loaded onto a DEAE-5 PW column (TSK) and eluted with a linear KCl gradient from 0.1 to 1M KCl in BC buffer. The Mediator complex eluted between 0.4 and 0.6M KCl. The fractions containing Mediator were pooled and dialyzed against BD700: BD buffer, (20 mM Hepes pH 7.5, 20 mM β-Mercaptoethanol, 0.2 mM PMSF, 0.2 mM EDTA, 10% glycerol, and 700 mM (NH4)2SO4). This fraction was then loaded onto a Phenyl-Sepharose Hydrophobic Interaction Chromatography (HIC) column and eluted with a linear reverse gradient from 0.7 to 0.025M (NH4)2SO4 in BD buffer. The Mediator complex eluted between 0.3 and 0.1M (NH4)2SO4. The Mediator-containing fractions were again pooled and dialyzed against BA100: BA buffer, pH 7.5+100 mM NaCl (20 mM Hepes, 20 mM B-Mercaptoethanol, 0.2 mM PMSF, 0.2 mM EDTA, 10% glycerol and 100 mM NaCl) and loaded onto a Heparin Agarose column. The column was washed with BA100 and step-eluted with BA buffer containing 0.25, 0.5, 1M and 1M NaCl. The Mediator complex eluted in the 0.5M NaCl (BA500) fraction. A portion of this fraction was then loaded on a Superose-6 (gel filtration column) that was equilibrated and run in BC100. The Mediator complex eluted from the gel filtration column with a mass range between 1-2MDa.

### Reconstituted In Vitro Transcription Assay

The reconstituted in vitro transcription by RNA polymerase II was performed as previously described (Flores et al., 1992; LeRoy et al., 2008, 2019; Orphanides et al., 1998) with some modifications. A 1000 bp template DNA (unlabeled or Cy-3 labeled at 3′ end) containing adenovirus major late promoter, five Gal4 binding sites, TATA-box sequence and 561 bp from eGFP sequence was used. First, pre-initiation complex was assembled at RT for 15 min by mixing the following components: 50 nM RNA polymerase II enriched for hypophosphorylated CTD, 50 nM general transcription factors (TFIIA-B-D-E-F-H), and 5.75 nM template DNA, in a buffer containing 10 mM HEPES pH 7.5, 65 mM NaCl, 6.25 mM MgCl2, and 6.25 mM Sodium butyrate. Next, 10 nM Mediator complex and 10 nM GAL4 (Gal4 DNA binding domain fused to activation domain of VP16) were added to the reaction. Last, nucleotide mix containing 0.375 mM ATP, CTP, UTP, GTP (Invitrogen), 0.01 U RNase Inhibitor (Invitrogen), 1.25% PEG-8000 were added together with one of the following: a) various amounts of purified exogenous Pou5f1 RNA (0-500 nM) b) spermine (Sigma, S4264) c) extra NTPs (Invitrogen) d) extra NaCl e) heparin (Sigma, H3393). The reaction was incubated at 30° C. for 2 hr. RNA isolation was performed using RNeasy kit (Qiagen) by including a spike-in RNA control and an RNA carrier. Purified RNAs were treated with ezDNase (Invitrogen) for 30 min at 37° C. to eliminate the template DNA. Reverse transcription was performed using Superscript IV (Invitrogen) and qPCR was performed with SYBR Green Real Time PCR master mix (Invitrogen) to quantify the template derived transcriptional output. The Ct values of the reactions were normalized to the spike-in RNA control. The concentration of template derived transcriptional output was calculated by using a standard curve of qRT-PCR Ct values generated by known amounts of serially diluted GFP RNA. The sequence of primers used for qRT-PCR are indicated in Table S1.

To visualize the droplets formed in the reconstituted transcription assay, using a micropipette (2-20 μL), 5 μL of the reactions were loaded onto a homemade chamber, which was prepared by attaching coverslips to a glass slide by parallel strips of double-sided tape (Sabari et al., 2018). After the droplets were settled on the glass coverslip, the images were collected by using RPI Spinning Disk confocal microscope with a 100× objective. To account for camera artifacts in the images, brightfield Images of droplets from reconstituted assays were subjected to a white tophat filter with a disk element radius of 21 using the MorphoLib plugin in ImageJ, then a Gaussian filter (sigma=1) was applied.

### Constructing a Free-Energy for RNA-Protein Phase Behavior

The goal in this section is to develop a simplified and coarse-grained model that captures the qualitative physics of RNA-protein mixtures. Based on phenomenological observations of transcriptional proteins and RNA (FIG. 2), such a model must recapitulate the following key features:

Transcriptional proteins phase separate in the absence of RNA through other types of interactions, albeit at higher concentrations.

At fixed protein concentrations, addition of RNA initially promotes de-mixing and at higher levels drive a re-entry into the mixed phase.

Motivated by the evidence that transcriptional condensates recruit diverse coactivators, transcription factors, and other proteins of the transcriptional apparatus (Boija et al., 2018; Guo et al., 2019; Sabari et al., 2018; Shrinivas et al., 2019), an effective protein component P was defined that lumps together different transcriptional molecules. Similarly, while different species of RNA are likely present within these condensates, an effective RNA species (R) was defined.

### Landau Model

First, this problem was approached by constructing a phenomenological free-energy with 2 order-parameters that represent scaled concentrations of protein (ϕₚ(\vec{r}, t)) and RNA (ϕᵣ(\vec{r}, t)). The free-energy (normalized to k_{B}T=1) was defined as:

$$f [ {ϕ}_{p} , {ϕ}_{r} ] = {\int }_{V}$$

$${d}^{d} V ( {f}_{dw} ( {ϕ}_{p} ( \vec{r} , t ) ) + {ρ}_{r} {ϕ}_{r}^{2} + {χ}_{eff} ( {ϕ}_{p} ( \vec{r} , t ) , {ϕ}_{r} ( \vec{r} , t ) ) + \frac{κ}{2} {( ∇ {ϕ}_{p} )}^{2} )$$

Here, f_{dw}(ϕₚ(\vec{r},t)=ρₛ(ϕₚ−α)²(ϕₚ−β)² is a standard double-well potential that ensures protein components phase separate without RNA with co-existence concentrations specified by α, β. Choice of κ>0 ensures that there is finite surface tension for the protein condensate. The second-order term for RNA (ρᵣ>0) states that within this model-framework, RNA cannot phase-separate in the absence of protein. Given that electrostatic interactions at physiological salt conditions are fairly short-ranged (Debye length ˜1 nm), the non-linear nature of RNA-protein interactions was captured in an effective interaction term χ_{eff}. This interaction term was defined in the spirit of the Landau-Ginzburg approach as an expansion in powers of the order parameters:

$${χ}_{eff} ( {ϕ}_{p} , {ϕ}_{r} ) = - {χϕ}_{p} {ϕ}_{r} + a {ϕ}_{p} {ϕ}_{r}^{2} + b {ϕ}_{p}^{2} {ϕ}_{r}^{2} + c {ϕ}_{p}^{2} {ϕ}_{r}^{2} + … + H . O . T$$

While symmetry arguments often dictate or exclude certain types of terms (odd powers in Ising models for example) in such an expansion, there are no obvious symmetry constraints for this system. Hence, this modeling approach is to minimize the number of higher-order terms that need to be included to recapitulate the experimentally observed reentrant phase transition. These experimental results suggest that low concentrations of RNA promote phase separation, and thus the lowest order term (−χϕₚϕᵣ, χ>0) lowers the free-energy. However, higher-order terms must counter this and below the inventors outline how it was determined which terms to include. In general, the stability of a mixture described by such a free-energy can be ascertained from the Jacobian matrix J. For this model, the elements of this 2×2 matrix are:

$${J}_{pp} = \frac{{\partial }^{2} f}{\partial {ϕ}_{p}^{2}} = 2 {ρ}_{p} ( 6 {ϕ}_{p}^{2} - 6 {ϕ}_{p} ( β + α ) + {( α - β )}^{2} ) + 2 b {ϕ}_{r} + 2 c {ϕ}_{r}^{2} {J}_{pr} = \frac{{\partial }^{2} f}{\partial {ϕ}_{p} \partial {ϕ}_{r}} = - χ + 2 a {ϕ}_{r} + 2 b {ϕ}_{p} + 4 c {ϕ}_{p} {ϕ}_{r}$$

$${J}_{rr} = \frac{{\partial }^{2} f}{\partial {ϕ}_{r}^{2}} = 2 {ρ}_{r} + 2 a {ϕ}_{p} + 2 c {ϕ}_{p}^{2}$$

The mixed phase is no longer stable to perturbations when at least one eigen value of J becomes negative (spinodal instability). In the absence of RNA, the spinodal satisfies Jₚₚ=0. If only the pair-wise interaction terms were considered (−χϕₚϕᵣ), the spinodal region broadens i.e., phase separation is promoted at lower protein concentrations when RNA is present. The effect of an additional higher-order term (only one of a, b or c is non-zero) on the Jacobian matrix was next characterized. Briefly, it was ascertained that:
a>0: While the free-energy is dominated by repulsive interactions at higher RNA concentrations, the Jacobian matrix predicts a continuous underlying instability. Instead of suppressing phase separation at higher RNA concentrations and promoting re-entry to dilute phase, this term would instead change the composition of the demixed phases.
b>0: While this term promotes a reentrant behavior, the resulting regions of instability demix RNA away from protein for most values of b.
c>0: For values of c that are not too large (i.e. c<≈ρᵣ), the resulting phase diagram mirrors a reentrant shape with RNA enrichment in the protein condensate. If c is moderately large, then a second de-mixing transition (similar to case 2 i.e. b>0) is observed at high values of ϕₚ, ϕᵣ. Since the inventors are interested in the limit of relatively low protein/RNA concentrations, and the values of ϕₚ, ϕᵣ represent qualitative proxies of protein/RNA concentrations, it was chosen to explore this model in this parameter regime.

While cubic and higher-order terms are required to recapitulate complete phase-behavior, this model was explored with c>0, assuming the coefficients a, b are small. In the simulations reported in FIGS. 4-6, the free-energy parameters are α=0.1, β=0.7, χ=1.0, c=10.0, κ=0.5, ρₛ=1.0, ρᵣ=10.0, a=b=0. All free-energy calculations were performed with Python and code is available at:

github.com/krishna-shrinivas/2020_Henninger_Oksuz_Shrinivas_RNA_feedback.

### Flory-Huggins Model

In this approach, rather than employ a phenomenological model, a microscopic model motivated by Flory-Huggins polymer-solution theory (Flory, 1942) was parameterized. The simplified F-H model contains 3 components-protein, RNA, and the solvent (s), whose volume fractions are defined as ϕₚ(\vec{r}, t), ϕᵣ(\vec{r}, t), 1−ϕₚ(\vec{r}, t)−ϕᵣ(\vec{r},t) respectively. The free-energy (normalized as before) is defined as:

$$f = {\sum }_{i} \frac{{ϕ}_{i}}{{r}_{i}} log ( {ϕ}_{i} ) + {\sum }_{i , j > i} {χ}_{ij} {ϕ}_{i} {ϕ}_{j}$$

Here, rᵢ are the solvent-equivalent polymerization lengths of the RNA & protein (assumed to be equal for simplicity) and χᵢⱼ are the various pairwise interaction terms. As before, it was assumed that these interactions to be short-ranged at physiological salt levels. Choice of χₚᵣ>χₚₛ>0 and χᵣₛ<0 recapitulate the attractive contributions of protein-protein/protein-RNA interactions and repulsive RNA-RNA interactions. With these choices of constraints, the resulting free-energy looks similar to the phase diagram from the Landau approach with c>0 (FIG. 13B) where the key F-H parameters are χₚᵣ=1.1, χₚₛ=0.75, χᵣₛ=−0.6, and rₚ=rᵣ=30.

### Numerical Phase-Field Simulations

Numerical investigations of the coupled-equations outlined in FIG. 4C were performed with the FiPy package (Guyer et al., 2009). Simulations were performed on a 2-D/3-D square lattice (Lₓ=L_{y}=200, dx=0.3; Lₓ=L_{y}=L_{z}=40, dx=1.0) and with adaptive time-stepping (dtₘᵢₙ=1e-8, dtₘₐₓ=5e-1) until steady state is reached (which typically requires ˜10000 simulation steps).

The chemical potential for the protein components is calculated as:

$${μ}_{p} = \frac{df}{d {ϕ}_{p}} = 2 {ρ}_{s} ( {ϕ}_{p} - α ) ( {ϕ}_{p} - β ) ( 2 {ϕ}_{p} - α - β ) + κ {∇}^{2} {ϕ}_{p} - {χϕ}_{r} + 2 c {ϕ}_{r}^{2} {ϕ}_{p}$$

The radius of condensates was inferred from the volume of mesh regions where

$${ϕ}_{p} \geq \frac{α + β}{2} .$$

The mobility of RNA and protein were chosen to be 1.0 unless mentioned elsewhere.

### Design of Simulations to Vary RNA Features and Rates of RNA Synthesis

The inventors designed simulations (FIG. 6E) to study the effect of RNA features and rates of effective synthesis on condensate size. The rates of synthesis were changed by increasing kₚ by multiplicative factors (see x-axis in FIG. 6E). Since RNA length is not explicitly incorporated in the model framework, the effective local synthesis rates of longer RNA was defined as a product of kₚ and an additional multiplicative factor (1,2, and 4× for short, medium, and long RNA respectively) to mimic increased local concentrations of RNA.

### Calculation of Number of Charged Molecules in Condensates

In estimating the number and charge of transcriptional proteins (FIG. 8), previous estimates (Cho et al., 2018) that suggest key transcriptional proteins such as Mediator are present at 10-100 molecules in transcriptional condensates were used. Further, molecules such as MED1 or BRD4 contain large disordered domains with net positive charge of +5 to to+40. This provides a highly approximate estimate of 25-500 as the effective positive charge. Since there are many more transcriptional proteins and most proteins tend to contain net positive charges, it is likely that this estimate represents lower bounds on the range. Steady-state levels of nascent eRNA (FIG. 8) suggest a range of 0.2-10 molecules, and since super-enhancers typically contain clusters of such active enhancers, the typical range of eRNA molecules at a transcriptional condensate was approximated as between 1-10. Since RNA carries a charge of around −1 per nt (Banerjee et al., 2017) and eRNAs are short (<1 kb), the effective negative charge during initiation was estimated to be in the range 10-1000. During productive elongation, mRNAs are produced in bursts ranging from few to tens (1-50) and are typically longer (>1 kb), suggesting a conservative estimate of the effective charge to range from (1000-100,000). It is important to stress that these approximations are performed with the aim of obtaining order-of-magnitude estimates and do not account for factors such as local composition of different proteins or extent to which nascent mRNAs may be coated by RNA-binding proteins. With the above numbers, concentrations were estimated based on a typical transcriptional condensate of size r=0.25 μm (Cho et al., 2018) that suggests that eRNA concentrations range about 10-200 nM and transcriptional proteins range 1-20 μM within the condensate.

### Reactive/Diffusive Time-Scales and Estimates in Cells

As defined in the model (FIG. 4B), the key rates of synthesis/degradation reactions are kp/kd, which have units of s⁻¹, and thus the relevant time-scales are tᵣ=kₚ⁻¹(or k_{d}⁻¹). Timescales of RNA transport depend on both the diffusivity as well as the size of the condensate (L) and is defined as t_{d}=L²/Mᵣₙₐ The range of diffusivity of the nascent transcript was approximated at the lower end by the diffusivity of chromatin, which ranges from 10^{-3.5}-10⁻² μm²/s (Gu et al., 2018) and on the higher end by those of freely diffusing mRNPs, which can be upto 5×10⁻² μm²/s (Niewidok et al., 2018). By assuming a typical eRNA of size 100 nt and Pol II transcription rates as ˜20-70 nt/s (Maiuri et al., 2011) typical synthesis rates of ˜0.5 eRNA s⁻¹Pol II⁻¹ were inferred. In previous work (Cho et al., 2018), it was seen that clusters that contain multiple polymerases (>5), are typically around r≈200-400 nm. Since super-enhancers typically contain clusters of enhancers with multiple sites of eRNA synthesis (˜5), this gives an effective synthesis rate of kₚ≈2.5 s⁻¹Pol II⁻¹. This allows one to approximately obtain the ratio of diffusive and reactive time-scales as

$$\frac{{t}_{d}}{{t}_{r}} = \frac{{kr}^{2}}{M} \approx 2 - 1000$$

over the range of parameters including diffusivity and radii of cluster.

### Calculation of Charge Balance

Charge-balance calculations were performed (FIGS. 2, 9, 10 and 11) employing the following method. Net protein charge per molecule was calculated as Cₚ=#(R, K)−#(D, E) for the relevant sequence including the GFP tag. RNA charge per molecule was calculated as Cᵣ=−(# of bp), assuming an approximate charge of −1 per nucleotide (Lin et al., 2019). Next, the charge balance ratio was computed at a particular RNA and protein concentration as:

$$Charge - balance ratio = \frac{min ( {C}_{p} [ P ] , {C}_{r} [ R ] )}{max ( {C}_{p} [ P ] , {C}_{r} [ R ] )}$$

The effective concentration of MED1-IDR in the assays was 1000 nM. The results were not quantitatively affected by inclusion/exclusion of the partial charge on Histidine residues, partly due to their low frequency on the protein sequences. For Heparin, a charge of roughly −3 per monomer was employed (Lin et al., 2020) and for single-stranded DNA, a charge of −1 per nt was employed. A comprehensive listing of charges of various species employed in this study are provided in Table S2. The fit is quantified by calculating Pearson correlation coefficient (r) was calculated between the median droplet partition value at different concentrations and the relevant charge-balance ratios and reported in FIGS. 2, 9, 10, and 11. A higher correlation implies that experimental data follow a similar qualitative trend as the estimated charge-balance curves. The code for performing these calculations are available at:

github.com/krishna-shrinivas/2020_Henninger_Oksuz_Shrinivas_RNA_feedback.

### Transcription Inhibition by Small Molecules

For small molecule inhibition experiments, cells were treated with 100 μM DRB (Sigma), or 1 μM Actinomycin-D (Sigma) in 2i media (detailed above) for 30 minutes, then imaged. For wash-out experiments, media was replaced with fresh 2i media and cells were allowed to recover for 1 hour, then the cells were imaged.

### Condensate Size

Cells with endogenously-tagged Med1-GFP (Sabari et al., 2018) were plated on glass-bottom dishes (Mattek) coated with poly-L-ornithine (Sigma) and laminin (ThermoFisher). Mock (DMSO) and treated cells were imaged on a LSM 880 Confocal Microscope with Airyscan to obtain super-resolution z-stacks for at least 8 different fields containing multiple cells. For quantification, a manual threshold was applied equally across all conditions to remove background, and the size of Med1-GFP puncta was quantified in 3D using the 3D object counter plugin (Fiji/ImageJ).

### Condensate Lifetime

HaloTag was endogenously knocked into 5′-end of Med19 via homology-directed repair (HDR) in mouse embryonic stem cells (R1 mESCs). Three single-guide RNAs (sgRNAs) targeting+/−100 bps from the start codons of Med19 gene were designed using the web-based CRISPR Design tool (http://crispr.mit.edu) and integrated into a Streptococcus pyogenes Cas9 vector (Addgene #62988) for standard CRISPR/Cas9 editing. Single positive colonies were sorted by fluorescence-activated cell sorting (FACS) and validated under the microscope.

Cells were cultured in serum-free 2i medium on poly-L-ornithine (PLO) and Laminin-coated flasks for more than two days and then were transferred onto coated imaging dishes for another day. Before imaging, cells were stained with (PA)-JF549-HaloTAG dye (a gift from Luke Lavis Lab, Janelia Research Campus) of 100 nM concentration for 2 hours followed by a 60-minute wash in fresh 2i medium. Lastly, dishes were filled in with 2 ml Leibovitz's L-15 Medium (no phenol red, Thermo Fisher) and brought to the microscope for imaging.

Photo-activation localization microscopy (PALM) imaging was performed using a Nikon Eclipse Ti microscope with a 100× oil immersion objective (NA 1.40) (Nikon, Tokyo, Japan). A 405 nm beam of 100 mW power (attenuated with 25% AOTF) and a 561 nm beam of 500 mW power were columnated and superposed to perform simultaneous activation and excitation. The combined beam was expanded and re-collimated with an achromatic beam expander (AC254-040-A and AC508-300-A, THORLABS) to improve the uniformity of illumination across the whole region of interest (ROI 256\hat{ }2 pixels). Images were acquired with an Andor iXon Ultra 897 EMCCD camera (gain 1000, exposure time 50 ms) interfaced through Micro Manager 1.4. 2400 frames were acquired for each imaging cycle. The cells were maintained at 37° C. in a temperature-controlled platform (In Vivo Scientific, St. Louis, MO) on the microscope stage during image acquisition. Med19-Halo cluster lifetimes were calculated as previously described using the qSR software (dark time tolerance=20 frames, min cluster size=50) (Andrews et al., 2018), and a cumulative distribution was generated using Prism software (GraphPad).

### Nascent RNA Imaging

For the nascent RNA experiments in FIGS. 14A-14C, 1.25×10⁵ wildtype mESCs were plated on coverslips coated with poly-L-ornithine (Sigma) and Laminin (ThermoFisher). After overnight plating, nascent RNA labeling with 2.5 mM EU was done with the Click-iT™ RNA Alexa Fluor™ 594 Imaging Kit (Thermofisher) according to manufacturer instructions for 10 minutes. After incubation, cells were immediately fixed with 4% paraformaldehyde for 10 minutes, washed 3× with PBS, then permeabilized with 0.5% TritonX-100 in PBS for 15 minutes. After the Click-iT reaction, coverslips were blocked with 4% RNase-free BSA in PBS for 10 minutes at room temperature. Coverslips were incubated with primary antibodies (1:500; rabbit Abcam ab64965 for MED1 and rat Millipore Sigma 04-1571 for Pol II-S2) in 4% BSA/PBS at room temperature overnight. The next day, coverslips were washed 3× with PBS, then incubated in secondary antibody (1:500; goat anti-rabbit AlexaFluor-488 Thermofisher A11008, goat anti-rat AlexaFluor-647 Invitrogen A21247) for 1 hour at room temperature. After washing 3× with PBS, coverslips were stained with 1:1000 Hoechst 33342 in PBS, incubated for 15 minutes at room temperature, washed 3× with PBS, and mounted on imaging slides with Vectashield Mounting Media. Images were collected on the RPI Spinning Disk confocal. Representative images in FIG. 14A are single z-planes of median-subtracted (px=10) and Gaussian smoothed (sigma=1) channels to correct for uneven illumination and background.

For analysis of these images, nuclei were segmented using the Cellpose algorithm (Stringer et al., 2020) on the 405 Hoechst channel images. For average image analysis in FIG. 14B, all channel images were maximally projected, subtracted by median filter (px=10), and Gaussian smoothed (sigma=1). The center of MED1 and Pol II-S2 puncta were segmented as follows. The Laplace of Gaussian transformation (sigma=3) was applied to the images using the scikit-image package in python, and puncta were identified above a threshold intensity 3 standard deviations above the mean of the image. All spots were confirmed to be in nuclei. A 1 μm by 1 μm box was centered on the spots, and the box subimage was collected for that region in both the processed MED1 and Pol II-S2 channel images. These subimages from >10 imaged fields were stacked and averaged, which was the input for the contour plots in FIG. 14B. Radial intensity plots in FIG. 14C show the distribution of these averaged signals as a function of the distance from the center of the spot, along with their correlation to EU RNA signal.

### Reporter Assay to Determine the Effect of Local RNA Synthesis on Transcription

Vectors used in the reporter assay are modified from pTETRIS-cargo vector, gift from J. M. Calabrese (Kirk et al., 2018). 6× STOP codon sequence was cloned into NotI digested pTETRIS-cargo vector using Gibson cloning strategy by following the manufacturer's instructions (NEB). This vector is called pTETRIS-cargo-STOP. The feedback gene and the reporter gene have their own polyA termination signal (200-300 bp) to terminate transcription. There is 51 bp between these two polyA signals that are facing each other. The reporter gene is regulated by a phosphoglycerate kinase (PGK) promoter. Various versions of the pTETRIS-cargo-STOP using Gibson cloning strategy (NEB): i) the relative orientations of the feedback RNA and luciferase reporter were altered (tandem or divergent orientations) ii) feedback RNAs and luciferase reporter were cloned into separate vectors. Using Gibson cloning strategy (NEB), various RNA sequences were cloned downstream of the 6× STOP sequence to prevent translation of these RNAs. Stable cell lines for individual RNAs were generated by transfecting Med1-GFP mESCs with the following vectors: 1.0 μg pTETRIS-cargo-STOP containing individual RNAs, 1.0 μg rTTA-cargo, gift from J. M. Calabrese (Kirk et al., 2018), and 1 μg piggyBAC transposase (Systems Biosciences). Cells were selected on puromycin (2 μg/ml) and G418 (200 μg/ml) for 1 week for successful integrations. For luciferase assays, 1×10⁵ cells of each genotype were plated in triplicate on 0.2%-gelatin-coated 24-well plates and allowed to settle overnight. Cells were treated with doxycycline (Sigma) and harvested after 24 h to measure either luciferase activity or to purify RNA. Luciferase activity was measured using the Luciferase Assay System (Promega) according to manufacturer instructions. Luciferase signal was normalized to total protein content, measured by BCA protein assay kit (Invitrogen, #23227), and then normalized to a control not treated with doxycycline. To measure RNA expression, RNA was purified using the Qiagen RNeasy Mini kit (Qiagen) according to manufacturer instructions, cDNA was generated by Superscript III (Invitrogen) according to manufacturer instructions, and 10 ng of cDNA was used in a qRT-PCR SYBR-green reaction (Life Technologies) with primers specific to a common sequence shared across the vectors (qPCR_Tetris, Table S1). Ct values were normalized to a housekeeping gene (qPCR_mActb, Table S1) and a control condition with no doxycycline treatment.

For the washout experiments in FIG. 14I, reporter cells were plated as described above. After 24 hours of dox treatment, media was replaced with fresh media, whereas control cell media was replaced with dox-containing media. After an additional 24 hours, luciferase levels were measured as described above. For the antisense oligo experiments of FIG. 14J, antisense oligos (LNA gapmers, Qiagen) were designed using the Qiagen GeneGlobe tool against the feedback RNA. A negative scrambled control was also included. Reporter cells were plated as described above in triplicate, and cells were transfected with 25 nM ASO with Lipofectamine-3000 (and no P3000 enhancer agent). After overnight transfection, cell media was replaced with dox-containing or fresh 2i media as a control. After 24 hour dox treatment, RNA and luciferase levels were quantified by qRT-PCR and luminescence, respectively, as described above. For the analysis of luciferase rescue, luminescence values of the dox conditions were first normalized to the no dox condition for that ASO, and then normalized to the dox condition of the negative scrambled control.

For imaging experiments in FIGS. 6C-6D, the reporter construct was modified using Gibson cloning to include a 24×-MS2 hairpin (Cho et al., 2018) at the 5′ end of the RNA sequence (2,456 nt total). Cell lines with this construct and double MS2 capsid protein fused to an mCherry tag (2×MCP-mCherry) were generated as detailed above in a mESC background with endogenously-tagged Med1-GFP (Sabari et al., 2018). 1×10⁶ reporter cells were plated on glass-bottom dishes (Mattek) coated with poly-L-ornithine (Sigma) and Laminin (ThermoFisher) After overnight plating, cells were treated with 10, 100, or 1000 ng/mL doxycycline for 24 hours. Cells were imaged on an RPI Spinning Disk Confocal with the following laser powers and exposure times: 488 70% 500 ms, 561 40% 300 ms. Images were maximum projected, median subtracted (px=10), and Gaussian filtered (sigma=1) to correct for uneven illumination and background subtraction. For analysis of these images in FIG. 6D, nuclei were segmented using the Cellpose algorithm (Stringer et al., 2020) on images from the 561 channel that had been subjected to a maximum and median filter (px=10). For average image analysis, both the RNA and MED1-GFP channel images were maximally projected, subtracted by median filter (px=10), and Gaussian smoothed (sigma=1). The centers of RNA spots in a maximum projection of the 561 channel were manually marked using ImageJ. All spots were confirmed to be in nuclei. A 1 μm by 1 μm box was centered on the RNA spot, and the box subimage was collected for that region in both the processed RNA and MED1-GFP channel images. These subimages from >10 imaged fields were stacked and averaged, which was the input for the contour plots in FIG. 6D. Radial intensity plots in FIG. 14D show the distribution of these averaged signals as a function of the distance from the center of the spot. To control for global Dox effects, the size, number, and partition ratio of MED1-GFP condensates in all conditions were quantified by using a threshold of 3 standard deviations above the mean intensity of the image to segment condensates. Partition ratio for each condensate was calculated as the average intensity inside the condensate divided by the average intensity of the nucleoplasm.

### Supplemental Information

TABLE S1
Oligonucleotides used in this study
Primers used to amplify templates for in vitro
transcription assay

The following sequences were added to the forward
and reverse primers to add the bacterial polymerase
promoters:
T7 (add to 5′ of sense or forward primer):
5′-TAATACGACTCACTATAGGG-3′ (SEQ ID NO: 1)
SP6 (add to 5′ of antisense or reverse primer):
5′-ATTTAGGTGACACTATAGAA-3′ (SEQ ID NO: 2)

| Primer ID | Sequence (5′ to 3′) |
|---|---|
| txn_eRNA_Oct4_002_F | GGCCTAGACAGCACTCTCCA (SEQ ID NO: 3) |
| txn_eRNA_Oct4_002_R | TGGATCTCTGTGAGTTCAAG (SEQ ID NO: 4) |
| txn_eRNA_Trim28_002_F | AAATCTTGGAGAGAGTAGGA (SEQ ID NO: 5) |
| txn_eRNA_Trim28_002_R | GGGAAAAAGTTACAGTGACC (SEQ ID NO: 6) |
| txn_eRNA_Oct4_003_F | CTTCCAGAACATCTGGATTT (SEQ ID NO: 7) |
| txn_eRNA_Oct4_003_R | AAAACAAACAAAAAAGAGTC (SEQ ID NO: 8) |
| txn_eRNA_Nanog_002_F | AGCCTGCCTTTTGGCTACCA (SEQ ID NO: 9) |
| txn_eRNA_Nanog_002_R | AGAGTGCCAGGTCCCCTGGA (SEQ ID NO: 10) |
| trx_pre-mRNA_Oct4_500_F | TAGGTGAGCCGTCTTTCCACC (SEQ ID NO: 11) |
| trx_pre-mRNA_Oct4_500_R | CCCAATTCCCTTCACTGCTGC (SEQ ID NO: 12) |
| trx_pre-mRNA_Trim28_500_F | CGGGCGGTGAGAAGCGT (SEQ ID NO: 13) |
| trx_pre-mRNA_Trim28_500_R | AATGCATGCACACCCTCTGATT (SEQ ID NO: 14) |

Primers used to calculate number of RNA molecules in cells

| | |
|---|---|
| qPCR_15_eRNA_Trim28_002_F | AGAGGCTCTTCTGGGGTTGT (SEQ ID NO: 15) |
| qPCR_16_eRNA_Trim28_002_R | GCGAACAAGTAGGGCCAGTT (SEQ ID NO: 16) |
| qPCR_19_eRNA_Trim28_002_F | GCCCTGGATTGTACCTGTCC (SEQ ID NO: 17) |
| qPCR_20_eRNA_Trim28_002_R | ACCTTCAAAGTGGGTAACGCT (SEQ ID NO: 18) |
| qPCR_45_eRNA_Oct4_002_F | CAGGTTAGCCCTAAGCGTGC (SEQ ID NO: 19) |
| qPCR_46_eRNA_Oct4_002_R | AGGCTAGGGCACATCTGTTT (SEQ ID NO: 20) |
| qPCR_59_eRNA_Oct4_002_F | CCCTAAGCGTGCCTAGAGTAT (SEQ ID NO: 21) |
| qPCR_60_eRNA_Oct4_002_R | ACCAGGCTAGGGCACATCT (SEQ ID NO: 22) |
| qPCR_Trim28_intron_F3 | GCTGCTGCCCTGTCTACATT (SEQ ID NO: 23) |
| qPCR_Trim28_intron_R3 | CTGGCCACCCAGTACTCACT (SEQ ID NO: 24) |
| qPCR_Trim28_intron_F4 | GAGTACTGGGTGGCCAGGT (SEQ ID NO: 25) |
| qPCR_Trim28_intron_R4 | CCCCCTCTTAAACCAGCAG (SEQ ID NO: 26) |
| qPCR_Oct4_intron_F3 | GTTGGAGAAGGTGGAACCAA (SEQ ID NO: 27) |
| qPCR_Oct4_intron_R3 | CCCAATTCCCTTCACTGCT (SEQ ID NO: 28) |
| qPCR_Oct4_intron_F4 | AGAGGGAACCTCCTCTGAGC (SEQ ID NO: 29) |
| qPCR_Oct4_intron_R4 | CAGCCAAGTCCCTTTCACTT (SEQ ID NO: 30) |
| qPCR_mActb_F | GATCTGGCACCACACCTTCT (SEQ ID NO: 31) |
| qPCR_mActb_R | TGGGGTGTTGAAGGTCTC (SEQ ID NO: 32)A |
| qPCR_Tetris_5′F1 | AGAATTCGAGCTCGGTAC (SEQ ID NO: 33) |
| qPCR_Tetris_5′R1 | GCgaattcCTAGTTAGCTAG (SEQ ID NO: 34) |
| qPCR_Tetris_Luc_early_5′F | TTGCTCACGAATACGACGGT (SEQ ID NO: 35) |
| qPCR_Tetris_Luc_early_5′R | CTGTACATCGGTGTGGCTGT (SEQ ID NO: 36) |
| qPCR_Tetris_Luc_late_5′F | AAGAAGTGCTCGTCCTCGTC (SEQ ID NO: 37) |
| qPCR_Tetris_Luc_late_5′R | TACGTTAACAACCCCGAGGC (SEQ ID NO: 38) |
| qPCR_Tetris_Puro_5′F | GCTCGTAGAAGGGGAGGTTG (SEQ ID NO: 39) |
| qPCR_Tetris_Puro_5′R | CACCAGGGCAAGGGTCTG (SEQ ID NO: 40) |

Primers used to quantify transcriptional output from
reconstituted in vitro transcription assay

| | |
|---|---|
| qPCR_19_eRNA_Trim28_002_F | GCCCTGGATTGTACCTGTCC (SEQ ID NO: 41) |
| (Spike-in control) | |
| qPCR_20_eRNA_Trim28_002_R | ACCTTCAAAGTGGGTAACGC (SEQ ID NO: 42)T |
| (Spike-in control) | |
| GFP_qPCR_Fw (GFP primer) | cctgaagttcatctgcacca (SEQ ID NO: 43) |
| GFP_qPCR_Rv (GFP primer) | gtcttgtaggtgccgtcgtc (SEQ ID NO: 44) |

TABLE S2
Lengths and charges of nucleic acids and proteins used in this
study

| Nucleic | | Length | | |
|---|---|---|---|---|
| acids | Strand sense | (bp) | Charge | Sequence |
| Nanog | plus | 2268 | −2268 | AGCCTGCCTTTTGGCTACCAGCCA |
| enhancer | | | | CCTCTTCGCTCGGATCTTTCACCA |
| RNA | | | | GAGACTCTCAAAGACACTAAAGAG |
| | | | | GCAGGACAGGAATGGGGGTTGGGG |
| | | | | AGGGATCCATCGCCGTCTCCTAAG |
| | | | | CAGACTCCTTTGACCCGGAGCTGT |
| | | | | GCGCCCTGTACCAAACCTTTGTAG |
| | | | | AACTTGGGGTAAACTTAAGGCTAT |
| | | | | GGTGGCCTTGACTCCGTGGACCCA |
| | | | | GAGGCAAGTTTCCTCCTTTAGAGG |
| | | | | ACTCGCATGCATTTTGTTTCTAAT |
| | | | | TTGAAATGAGAACCGGCTTAGAGC |
| | | | | TTGAACCAGCCAGTTCTCTGGACT |
| | | | | CCTCCCAGCTCTTACAATTCCTCT |
| | | | | CCCGGACGGTTCCTAGAAGACAAA |
| | | | | GGCAAGCTTACCAAAATTACGTCG |
| | | | | CCCTTGGGACACACCTAGGGTTCC |
| | | | | CTGGTGGCATCTTTTTTTTTTCAT |
| | | | | TATAAACAGGAGTAAATTTTTGTA |
| | | | | AGGGCAGAGCTGGTAGCTGAGGGA |
| | | | | GAGGAACCCTTTGGCCTAGTGAAG |
| | | | | GTAGTTTGCTGGGCTTTGTATCCC |
| | | | | CGCCCCCACCTCCCCCGAGAGAGA |
| | | | | GAGAGAGAGAGAGAGAGAGAGAGA |
| | | | | GAGAGAGAGAGAGAGAGAGAGAGA |
| | | | | GAGAGAGAGAGAGAGAGACTACGT |
| | | | | GGTTATTTCAAAAACTTGAGTGTG |
| | | | | GCAAAAGTATGTAACTGGGATTAG |
| | | | | TAAGCATTTCTTTCCTTAGTGAGA |
| | | | | TTGGAGTAGAGGGTGGGAAAGGAC |
| | | | | CTTAGAATCCTCGAATGTTGGGCT |
| | | | | TAGGAATGGGGAGACAAGAGCCAT |
| | | | | CACAGAATGCCTATTGTCCTTCAA |
| | | | | TATGTTAGCGATGGGCCCCGTGCT |
| | | | | TTAGATTTTAGGCTTGTATTTTCT |
| | | | | TTGTGTGTGTGTGTGTTTGTTTTG |
| | | | | TTTCTGTTTCTTTAGGCAGTCTGG |
| | | | | AGATCAGGCTGGCTTTCAACTCCC |
| | | | | TGTGATGCCCCTACCTCTCCTGAG |
| | | | | GTGTGAGTGGCAGAATGCTCAGCG |
| | | | | GGTAAAACACACTTGTATAAGTGC |
| | | | | AAGAGAACCAAGTTCCAAACATTG |
| | | | | TCCTCTGACTTCCTCAGGCATGCC |
| | | | | TTAGCTCTAACAAATAAAATTGAA |
| | | | | GAAAAGGATTCCAAACCACGGTGA |
| | | | | AGGTGCCACATCTTTGATCCCCTT |
| | | | | AAGGCAGACAGAAGTAGAGGGATC |
| | | | | CCTGGGAGTTCAAAGCCAGGTTGG |
| | | | | TCTGCATACTGAGTTGGCTAGCCA |
| | | | | GGAATACACACTGAGACTCTGTCT |
| | | | | TTAAAAAAAAAAAAGTGTGAAGAC |
| | | | | TGCTTTCTCTGTCCCAGCACTTGG |
| | | | | GAGAGAGGGAAAGAAACACAGCAG |
| | | | | CCAGCCTTGTCTACACAGTGAGTT |
| | | | | CCAGGCTAACCAGATCTGACATAG |
| | | | | TGAGGTCCTGACTCAAAATTAAAA |
| | | | | ATTGGCTACAGATAATACTGTAGC |
| | | | | CCTTGGTTAGTCCGAGTACTTAAC |
| | | | | TCAACATACCATTCTTCGTTTAAG |
| | | | | CAAACCACGTGAAAGACTTTTCAC |
| | | | | TGAAGGCTGCAAGTCTTAAAATGA |
| | | | | CTTTGGTGATGCCTGCCTGGACTG |
| | | | | TCTACCCTCTGGAGCAGACTTACA |
| | | | | AAGAATATTTTTTACTAAGCGCTG |
| | | | | CATAAACCTTGATATTTTGAACGG |
| | | | | CCTATTCATTCTTTGCCTAATGAC |
| | | | | AAGAATCACATCAGGGACATATTT |
| | | | | GTATTAGTCCAGCGAATAAGCAGA |
| | | | | AGGTAGAACAGTTATTCTTTTGTT |
| | | | | CTATGATTTCTACTTAGGGGCTTT |
| | | | | AGTCCATCTGTTTATTGTTTAAAA |
| | | | | CCTTCATATCTCACCAAGTAGTGG |
| | | | | TGGCCATCCCTTTAACCCCAGCAT |
| | | | | TCTGAGAGACAGAAGCAGGTGGGT |
| | | | | CTCTGTGAGTTCGAGGCCAGCCTG |
| | | | | GCCTACAGATCTAATTCCAGGATA |
| | | | | GCCGGGGCTACATAGAGAAACTCT |
| | | | | GTCTCAAAATAAATAAGTAAATAA |
| | | | | ATAAATAAGTAAATGATATATTTA |
| | | | | CATTATTTATTATATCACAAATTA |
| | | | | TATACGTGTTCTATAGAATATGTT |
| | | | | ACTATATACATATTTTTTTTGGTT |
| | | | | TTTCGAGACAGGGTTTCATATAGC |
| | | | | ATTAGCTTCATATATATAATTTAT |
| | | | | ATGCAAAAATATCTCTGAAAATGG |
| | | | | AATCACTGGAACCCAATTCTAAAA |
| | | | | GTATTGTTTTTTTTTGTTTTTAAA |
| | | | | GTTTCCTCATACCTCAAAGTTGTC |
| | | | | AGAGGAGGGCTTAAGAGATGGGCT |
| | | | | AGAGGGGCTGGAGAGATAGCTCAG |
| | | | | CAGTTAAGAGCTCTGGTTGCTCTT |
| | | | | CCAGAGGACCAGGGTTCAATTTCC |
| | | | | AGTACCTACATGACAGCTCATAAC |
| | | | | TATCTGTAATTTCAGTTCCAGGGG |
| | | | | ACCTGGCACTCT |
| | | | | (SEQ ID NO: 45) |
| Trim28 | minus | 594 | −594 | AAATCTTGGAGAGAGTAGGACCTG |
| enhancer | | | | AGCTGTTTCCTATTTCTACTACTT |
| RNA | | | | CCATAGACATCCATGGGTCTCCTT |
| | | | | TGGTTCTTACCTTAAACTGCAGTT |
| | | | | CTAGAGGCTCTTCTGGGGTTGTAA |
| | | | | ACTGCCCTTCCCCAACTCAGGAGC |
| | | | | TCCCATTCCCTCCCCTTCCTAAAC |
| | | | | TGGCCCTACTTGTTCGCTCTACTT |
| | | | | TATCTTGCCAGGGGTTGTAACAGC |
| | | | | CTCCAATCTGTCTTGGATCAGGAA |
| | | | | GCAGTTTCCATGGGGTATTTTAAG |
| | | | | TGTAAGTTCCCCGGTGAAATGGAA |
| | | | | ACAAAAAGAATAAGGTATTCTGGG |
| | | | | GACAGTGTAGGCCCACTAGGGTGT |
| | | | | AGCTTGTTTTGTAAACACAGGACC |
| | | | | TGAGTTTTGGACTTTGCCCTGGAT |
| | | | | TGTACCTGTCCCAGTGGGTTACCT |
| | | | | TAGATGACACTAGCTTCATAATGC |
| | | | | AAGAGGAGGTTAAGATGCTGGCTA |
| | | | | GCGTTACCCACTTTGAAGGTACTG |
| | | | | GAAAAGGAAGACCTGATTTACCCA |
| | | | | AAAGAGGGAGATCTCTGTGCTTCT |
| | | | | ACCGCTTTAATTTTTTTTTTCTTT |
| | | | | TTTTAAATATTTTTGCAGGACTGG |
| | | | | TCACTGTAACTTTTTCCC |
| | | | | (SEQ ID NO: 46) |
| Pou5f1 | minus | 477 | −477 | GGCCTAGACAGCACTCTCCACCAC |
| enhancer | | | | AGTTCAAGTATGCCTGCAGCCCAG |
| RNA-1 | | | | CAGTCCTGTCTGTATTCAATACCA |
| | | | | ACCTTGTCTTATGGATTGTGATTT |
| | | | | CTCTTTTGGTGACTCACTGGCCAG |
| | | | | GACAAGAGACATATTCTGAGTCCT |
| | | | | TTAACTGCCTAGTGACTGGCTTTG |
| | | | | CAACCAGGTTAGCCCTAAGCGTGC |
| | | | | CTAGAGTATAATACAGTCCTTAAC |
| | | | | AGCAACTTTGTCTGAAGTCCCAAG |
| | | | | TCTTCTTAGAACTAGCTTGAAACA |
| | | | | GATGTGCCCTAGCCTGGTCCCGAG |
| | | | | CTGTGAGCCTGGTGGCCCTGGAGA |
| | | | | TGGGACAGCAGACCTGTTGGCTCA |
| | | | | TCTGATCCAGTTTCTTGCCTCCTG |
| | | | | GGTCTTAGAAAATCATCAGACTAA |
| | | | | CTTTTGGGTTTGATTCAAGGGTTC |
| | | | | CCTACATAGCCTTAGATGGCTTGG |
| | | | | AACTCACTCTGTAGACCAGGCTGG |
| | | | | CCTTGAACTCACAGAGATCCA |
| | | | | (SEQ ID NO: 47) |
| Pou5f1 | plus | 855 | −855 | CTTCCAGAACATCTGGATTTGGGA |
| enhancer | | | | AGAGACGTTGCTGGTCCCAGGGCG |
| RNA-2 | | | | GCTGGGGGTTGGGGTTGGGGGAGG |
| | | | | GGGATGCTAACCAGCAAGGAAGCT |
| | | | | GTTCCTGGCTGGGGCAGGCCTGAC |
| | | | | TGAGCTCATGTCGCTGAAACTCCT |
| | | | | CATTTCTCCCTATGGCTTCATAGG |
| | | | | GAGACCCAGCCTGGATGCTAACAC |
| | | | | GAGTGATTTCCCTGCTCTAGTCTA |
| | | | | GTGTCCTCCGTGAGTCCATTTAAC |
| | | | | TGATCACCCAGTCTGTGAGGAGGT |
| | | | | GGCTGAACTCACAGTAAGAAAGCT |
| | | | | GTGGGGGTCAACGCCTATTGTTTG |
| | | | | TTTGTTTTGTTTTAGACAAGGTCT |
| | | | | CCTGCTGAGGCTGGCTCAAGCTGG |
| | | | | CCTGGAGGACTCTTGTGTTTAAGG |
| | | | | CTGGCCTTAAATTCTCTTTAAAAG |
| | | | | AAAATCATGTGTATATATGTGTGT |
| | | | | CCATGTAACTGCAGATGACCACAG |
| | | | | CAGCCAAGAGATTTCTGTTCTCCT |
| | | | | AGCTGTAAACCACCCAATATGGGT |
| | | | | GCTAGGAACAGAATTTTAAAAGGG |
| | | | | TCCTCTGAAAGAGCAATGTACACT |
| | | | | CAAATGCTGAGTTCTTTCCCAGCC |
| | | | | CCTAGCCTTGGACCTTTGTTCTTA |
| | | | | TCACTTCCAACGCCCAAGGGCAGG |
| | | | | CATTATAGGTGTGGCATTCCGCAT |
| | | | | CTGGCTTCCCAGGATACCTTTTCA |
| | | | | TGCTGGTGGACCATCTCTGGCTGG |
| | | | | GGACGTGTGGGCTTCTCTGCTGTC |
| | | | | TTTGGTTCTCCAGACAGAACTCCG |
| | | | | AGACAGATCTTGACTTGGTTCTAA |
| | | | | AATACAGGTGGTTTGTGGCAAGTT |
| | | | | AACGAATTTTAGCTCAAATTTGGG |
| | | | | GTATTTAAGATACCATGGTGACTC |
| | | | | TTTTTTGTTTGTTTT |
| | | | | (SEQ ID NO: 48) |
| Pou5f1 | plus | 490 | −490 | TAGGTGAGCCGTCTTTCCACCAGG |
| promoter | | | | CCCCCGGCTCGGGGTGCCCACCTT |
| RNA | | | | CCCCATGGCTGGACACCTGGCTTC |
| | | | | AGACTTCGCCTTCTCACCCCCACC |
| | | | | AGGTGGGGGTGATGGGTCAGCAGG |
| | | | | GCTGGAGCCGGGCTGGGTGGATCC |
| | | | | TCGAACCTGGCTAAGCTTCCAAGG |
| | | | | GCCTCCAGGTGGGCCTGGAATCGG |
| | | | | ACCAGGCTCAGAGGTATTGGGGAT |
| | | | | CTCCCCATGTCCGCCCGCATACGA |
| | | | | GTTCTGCGGAGGGATGGCATACTG |
| | | | | TGGACCTCAGGTTGGACTGGGCCT |
| | | | | AGTCCCCCAAGTTGGCGTGGAGAC |
| | | | | TTTGCAGCCTGAGGGCCAGGCAGG |
| | | | | AGCACGAGTGGAAAGCAACTCAGA |
| | | | | GGGAACCTCCTCTGAGCCCTGTGC |
| | | | | CGACCGCCCCAATGCCGTGAAGTT |
| | | | | GGAGAAGGTGGAACCAACTCCCGA |
| | | | | GGAGGTAAGTGAAAGGGACTTGGC |
| | | | | TGGGCTGGCAGAGGCAGCAGTGAA |
| | | | | GGGAATTGGG |
| | | | | (SEQ ID NO: 49) |
| Trim28 | plus | 475 | −475 | CGGGCGGTGAGAAGCGTCCGGCTG |
| promoter | | | | CTTCCTcagccgcggcggcctctg |
| RNA | | | | cagccgcgtcgtcccctgcGGGGG |
| | | | | GCGGTGGCGAGGCGCAGGAGCTTC |
| | | | | TGGAGCACTGCGGCGTGTGTCGCG |
| | | | | AGCGCCTGCGGCCCGAGCGGGATC |
| | | | | CTCGGCTGCTGCCCTGTCTACATT |
| | | | | CGGCCTGCAGTGCCTGCCTGGGCC |
| | | | | CCGCTACACCCGCCGCAGCGAATA |
| | | | | ATTCGGGGGATGGCGGCTCGGCGG |
| | | | | GCGACGGCGCTAGTGAGTACTGGG |
| | | | | TGGCCAGGTGCCCCTCCCCCTCCT |
| | | | | CGCAGCCCGTGCTCGGGACTGCGC |
| | | | | CTGTGCGAGAGTATGGGGGCCCGG |
| | | | | GTAGGGTTAAGTAGGCCTGCTGGT |
| | | | | TTAAGAGGGGGCGGGGAACGGGTC |
| | | | | CTGGCCTCTGCCAATGCCCGTTAC |
| | | | | CAGGTCTGGACACCGAGGTGCAGA |
| | | | | ATGTGATGGGAGATGTCAAGAAAT |
| | | | | CAGAGGGTGTGCATGCATT |
| | | | | (SEQ ID NO: 50) |
| ssDNA | plus | 117 | −117 | GGTTCTGCCGCAGGTGGATCCGGT |
| | | | | ATGTCCACCGCCACGACAGTCGCC |
| | | | | CCCGCGGGGATCCCGGCGACCCCG |
| | | | | GGCCCTGTGAACCCACCCCCCCCG |
| | | | | GAGGTCTCCAACCCCAGCAAG |
| | | | | (SEQ ID NO: 51) |
| Template | NA | 1000 | −2000 | ATGACCCTGCTGATTGGTTCGCTG |
| DNA for | | | | ACCATTTCCGGGTGCGGGACGGCG |
| IVT | | | | TTACCAGAAACTCAGAAGGTTCGT |
| | | | | CCAACCAAACCGACTCTGACGGCA |
| | | | | GTTTACGAGAGAGATGATAGGGTC |
| | | | | TGCTTCAGTAAGCCAGATGCTACA |
| | | | | CAATTAGGCTTGTACATATTGTCG |
| | | | | TTAGAACGCGGCTACAATTAATAC |
| | | | | ATAACCTTATGTATCATACACATA |
| | | | | CGATTTAGGTGACACTATAGAATA |
| | | | | CAAGCTTGCATGCCTGCAGGTCCT |
| | | | | CGGAGGACAGTACTCCGCTCGGAG |
| | | | | GACAGTACTCCGCTCGGAGGACAG |
| | | | | TACTCCGCTCGGAGGACAGTACTC |
| | | | | CGCTCGGAGGACAGTACTCCGACT |
| | | | | CTAGAGGATCCCCGGTGTTCCTGA |
| | | | | AGGGGGGCTATAAAAGGGGGTGGG |
| | | | | GGCGCGTTCGTCCTCACTCTCTTC |
| | | | | CCCTCCAAGCAAGGGCGAGGAGCT |
| | | | | GTTCACCGGGGTGGTGCCCATCCT |
| | | | | GGTCGAGCTGGACGGCGACGTAAA |
| | | | | CGGCCACAAGTTCAGCGTGCGCGG |
| | | | | CGAGGGCGAGGGCGATGCCACCAA |
| | | | | CGGCAAGCTGACCCTGAAGTTCAT |
| | | | | CTGCACCACCGGCAAGCTGCCCGT |
| | | | | GCCCTGGCCCACCCTCGTGACCAC |
| | | | | CCTGACCTACGGCGTGCAGTGCTT |
| | | | | CAGCCGCTACCCCGACCACATGAA |
| | | | | GCAGCACGACTTCTTCAAGTCCGC |
| | | | | CATGCCCGAAGGCTACGTCCAGGA |
| | | | | GCGCACCATCTCCTTCAAGGACGA |
| | | | | CGGCACCTACAAGACCCGCGCCGA |
| | | | | GGTGAAGTTCGAGGGCGACACCCT |
| | | | | GGTGAACCGCATCGAGCTGAAGGG |
| | | | | CATCGACTTCAAGGAGGACGGCAA |
| | | | | CATCCTGGGGCACAAGCTGGAGTA |
| | | | | CAACTTCAACAGCCACAACGTCTA |
| | | | | TATCACGGCCGACAAGCAGAAGAA |
| | | | | CGGCATCAAGGCGAACTTCAAGAT |
| | | | | CCGCCACAACGTCGAGGACGGCAG |
| | | | | CGTGCAGCTCGCCGACCACTACCA |
| | | | | GCAGAACACCCCCATC |
| | | | | (SEQ ID NO: 52) |
| Recombinant | | Length | | |
| proteins | Amino acids included | (aa) | Charge | Notes |
| mEGFP | SKGEELFTGVVPILVELDGDVNGH | 237 | −6 | Note: |
| | KFSVRGEGEGDATNGKLTLKFICT | | | Charge |
| | TGKLPVPWPTLVTTLTYGVQCFSR | | | computed |
| | YPDHMKQHDFFKSAMPEGYVQERT | | | as |
| | ISFKDDGTYKTRAEVKFEGDTLVN | | | #R/K- |
| | RIELKGIDFKEDGNILGHKLEYNF | | | #D/E |
| | NSHNVYITADKQKNGIKANFKIRH | | | |
| | NVEDGSVQLADHYQQNTPIGDGPV | | | |
| | LLPDNHYLSTQSKLSKDPNEKRDH | | | |
| | MVLLEFVTAAGITLGMDELYK | | | |
| | (SEQ ID NO: 53) | | | |
| MED1-IDR | EHHSGSQGPLLTTGDLGKEKTQKR | 626 | 43 | |
| | VKEGNGTSNSTLSGPGLDSKPGKR | | | |
| | SRTPSNDGKSKDKPPKRKKADTEG | | | |
| | KSPSHSSSNRPFTPPTSTGGSKSP | | | |
| | GSAGRSQTPPGVATPPIPKITIQI | | | |
| | PKGTVMVGKPSSHSQYTSSGSVSS | | | |
| | SGSKSHHSHSSSSSSSASTSGKMK | | | |
| | SSKSEGSSSSKLSSSMYSSQGSSG | | | |
| | SSQSKNSSQSGGKPGSSPITKHGL | | | |
| | SSGSSSTKMKPQGKPSSLMNPSLS | | | |
| | KPNISPSHSRPPGGSDKLASPMKP | | | |
| | VPGTPPSSKAKSPISSGSGGSHMS | | | |
| | GTSSSSGMKSSSGLGSSGSLSQKT | | | |
| | PPSSNSCTASSSSFSSSGSSMSSS | | | |
| | QNQHGSSKGKSPSRNKKPSLTAVI | | | |
| | DKLKHGVVTSGPGGEDPLDGQMGV | | | |
| | STNSSSHPMSSKHNMSGGEFQGKR | | | |
| | EKSDKDKSKVSTSGSSVDSSKKTS | | | |
| | ESKNVGSTGVAKIIISKHDGGSPS | | | |
| | IKAKVTLQKPGESSGEGLRPQMAS | | | |
| | SKNYGSPLISGSTPKHERGSPSHS | | | |
| | KSPAYTPQNLDSESESGSSIAEKS | | | |
| | YQNSPSSDDGIRPLPEYSTEKHKK | | | |
| | HKKEKKKVKDKDRDRDRDKDRDKK | | | |
| | KSHSIKPESWSKSPISSDQSLSMT | | | |
| | SNTILSADRPSRLSPDFMIGEEDD | | | |
| | DL | | | |
| | (SEQ ID NO: 54) | | | |
| MED1-IDR | EAASGSQGPLLTTGDLGAEATQAA | 627 | −50 | |
| RHK-A | VAEGNGTSNSTLSGPGLDSAPGAA | | | |
| | SATPSNDGASADAPPAAAAADTEG | | | |
| | ASPSASSSNAPFTPPTSTGGSASP | | | |
| | GSAGASQTPPGVATPPIPAITIQI | | | |
| | PAGTVMVGAPSSASQYTSSGSVSS | | | |
| | SGSASAASASSSSSSSASTSGAMA | | | |
| | SSASEGSSSSALSSSMYSSQGSSG | | | |
| | SSQSANSSQSGGAPGSSPITAAGL | | | |
| | SSGSSSTAMAPQGAPSSLMNPSLS | | | |
| | APNISPSASAPPGGSDALASPMAP | | | |
| | VPGTPPSSAAASPISSGSGGSAMS | | | |
| | GTSSSSGMASSSGLGSSGSLSQAT | | | |
| | PPSSNSCTASSSSFSSSGSSMSSS | | | |
| | QNQAGSSAGASPSANAAPSLTAVI | | | |
| | DALAAGVVTSGPGGEDPLDGQMGV | | | |
| | STNSSSAPMSSAANMSGGEFQGAA | | | |
| | EASDADASAVSTSGSSVDSSAATS | | | |
| | ESANVGSTGVAAIIISAADGGSPS | | | |
| | IAAAVTLQAPGESSGEGLAPQMAS | | | |
| | SANYGSPLISGSTPAAEAGSPSAS | | | |
| | ASPAYTPQNLDSESESGSSIAEAS | | | |
| | YQNSPSSDDGIAPLPEYSTEAAAA | | | |
| | AAAEAAAVADADADADADADADAA | | | |
| | ASASIAPESWSASPISSDQSLSMT | | | |
| | SNTILSADAPSALSPDFMIGEEDD | | | |
| | DLM | | | |
| | (SEQ ID NO: 55) | | | |
| BRD4-IDR | CLRKKRKPQAEKVDVIAGSSKMKG | 678 | 18 | |
| | FSSSESESSSESSSSDSEDSETEM | | | |
| | APKSKKKGHPGREQKKHHHHHHQQ | | | |
| | MQQAPAPVPQQPPPPPQQPPPPPP | | | |
| | PQQQQQPPPPPPPPSMPQQAAPAM | | | |
| | KSSPPPFIATQVPVLEPQLPGSVF | | | |
| | DPIGHFTQPILHLPQPELPPHLPQ | | | |
| | PPEHSTPPHLNQHAVVSPPALHNA | | | |
| | LPQQPSRPSNRAAALPPKPARPPA | | | |
| | VSPALTQTPLLPQPPMAQPPQVLL | | | |
| | EDEEPPAPPLTSMQMQLYLQQLQK | | | |
| | VQPPTPLLPSVKVQSQPPPPLPPP | | | |
| | PHPSVQQQLQQQPPPPPPPQPQPP | | | |
| | PQQQHQPPPRPVHLQPMQFSTHIQ | | | |
| | QPPPPQGQQPPHPPPGQQPPPPQP | | | |
| | AKPQQVIQHHHSPRHHKSDPYSTG | | | |
| | HLREAPSPLMIHSPQMSQFQSLTH | | | |
| | QSPPQQNVQPKKQELRAASVVQPQ | | | |
| | PLVVVKEEKIHSPIIRSEPFSPSL | | | |
| | RPEPPKHPESIKAPVHLPQRPEMK | | | |
| | PVDVGRPVIRPPEQNAPPPGAPDK | | | |
| | DKQKQEPKTPVAPKKDLKIKNMGS | | | |
| | WASLVQKHPTTPSSTAKSSSDSFE | | | |
| | QFRRAAREKEEREKALKAQAEHAE | | | |
| | KEKERLRQERMRSREDEDALEQAR | | | |
| | RAHEEARRRQEQQQQQRQEQQQQQ | | | |
| | QQQAAAVAAAATPQAQSSQPQSML | | | |
| | DQQRELARKREQERRRREAMAATI | | | |
| | DMNFQS | | | |
| | (SEQ ID NO: 56) | | | |
| POU5F1 | MAGHLASDFAFSPPPGGGGDGPGG | 360 | −5 | |
| | PEPGWVDPRTWLSFQGPPGGPGIG | | | |
| | PGVGPGSEVWGIPPCPPPYEFCGG | | | |
| | MAYCGPQVGVGLVPQGGLETSQPE | | | |
| | GEAGVGVESNSDGASPEPCTVTPG | | | |
| | AVKLEKEKLEQNPEESQDIKALQK | | | |
| | ELEQFAKLLKQKRITLGYTQADVG | | | |
| | LTLGVLFGKVFSQTTICRFEALQL | | | |
| | SFKNMCKLRPLLQKWVEEADNNEN | | | |
| | LQEICKAETLVQARKRKRTSIENR | | | |
| | VRGNLENLFLQCPKPTLQQISHIA | | | |
| | QQLGLEKDVVRVWFCNRRQKGKRS | | | |
| | SSDYAQREDFEAAGSPFSGGPVSF | | | |
| | PLAPGPHFGTPGYGSPHFTALYSS | | | |
| | VPFPEGEAFPPVSVTTLGSPMHSN | | | |
| | (SEQ ID NO: 57) | | | |
| GAL4- | MKLLSSIEQACDICRLKKLKCSKE | 232 | −16 | Fusion |
| VP16 | KPKCAKCLKNNWECRYSPKTKRSP | | | of |
| | LTRAHLTEVESRLERLEQLFLLIF | | | (yeast) |
| | PREDLDMILKMDSLQDIKALLTGL | | | Gal4 |
| | FVQDNVNKDAVTDRLASVETDMPL | | | DNA |
| | TLRQHRISATSSSEESSNKGQRQL | | | binding |
| | TVSPEFPGIWAPPTDVSLGDELHL | | | domain |
| | DGEDVAMAHADALDDFDLDMLGDG | | | with |
| | DSPGPGFTPHDSAPYGALDMADFE | | | (Herpes |
| | FEQMFTDALGIDEYGG | | | Virus) |
| | (SEQ ID NO: 58) | | | VP16 |
| | | | | activation |
| | | | | domain. |
| Purified | | | | |
| protein | | Length | | |
| complexes | UniProt ID | (aa) | Charge | |
| TFIIA p55 | P52655 | 376 | −34 | TFIIA |
| TFIIA p15 | P52657 | 109 | 0 | TFIIA |
| TFIIB | Q00403 | 316 | 5 | TFIIB |
| TBP | P20226 | 339 | 13 | TFIID |
| TFIIE p56 | P29083 | 439 | −35 | TFIIE |
| TFIIE p34 | P29084 | 291 | 16 | TFIIE |
| TFIIF | P35269 | 517 | 0 | TFIIF |
| Rap74 | | | | |
| TFIIF | P13984 | 249 | 7 | TFIIF |
| Rap30 | | | | |
| ERCC3 | P19447 | 782 | −2 | TFIIH |
| ERCC2 | P18074 | 760 | −3 | TFIIH |
| p62 | P32780 | 548 | 7 | TFIIH |
| p52 | Q92759 | 462 | 6 | TFIIH |
| p44 | Q13888 | 395 | −7 | TFIIH |
| CDK7 | P50613 | 346 | 4 | TFIIH |
| Cyclin | P51946 | 323 | −1 | TFIIH |
| H | | | | |
| p34 | Q13889 | 308 | −1 | TFIIH |
| MAT1 | P51948 | 309 | −6 | TFIIH |
| Rpb1 | P24928 | 1970 | −3 | RNA Pol II |
| Rpb2 | P30876 | 1174 | −8 | RNA Pol II |
| Rpb3 | P19387 | 275 | −17 | RNA Pol II |
| Rpb5 | P19388 | 210 | −4 | RNA Pol II |
| Rpb7 | P62487 | 172 | −5 | RNA Pol II |
| Rpb6 | P61218 | 127 | −20 | RNA Pol II |
| Rpb4 | O15514 | 142 | −11 | RNA Pol II |
| Rpb8 | P52434 | 150 | −12 | RNA Pol II |
| Rpb9 | P36954 | 125 | −9 | RNA Pol II |
| Rpb11 | P52435 | 117 | −3 | RNA Pol II |
| Rpb10 | P62875 | 67 | 1 | RNA Pol II |
| Rpb12 | P53803 | 58 | 5 | RNA Pol II |
| MED1 | Q15648 | 1581 | 19 | Mediator |
| MED4 | Q9NPJ6 | 270 | −14 | Mediator |
| MED6 | O75586 | 246 | 3 | Mediator |
| MED7 | O43513 | 233 | -10 | Mediator |
| MED8 | Q96G25 | 268 | 0 | Mediator |
| MED9 | Q9NWA0 | 146 | 0 | Mediator |
| MED10 | Q9BTT4 | 135 | -3 | Mediator |
| MED11 | Q9P086 | 117 | -2 | Mediator |
| MED14 | O60244 | 1454 | 22 | Mediator |
| MED23 | Q9ULK4 | 1368 | -3 | Mediator |
| MED15 | Q96RN5 | 788 | 16 | Mediator |
| MED24 | O75448 | 989 | -6 | Mediator |
| MED16 | Q9Y2X0 | 877 | -1 | Mediator |
| MED25 | Q71SY5 | 747 | 5 | Mediator |
| MED17 | Q9NVC6 | 651 | -1 | Mediator |
| MED26 | O95402 | 600 | 13 | Mediator |
| MED18 | Q9BUE0 | 208 | -4 | Mediator |
| MED19 | A0JLT2 | 244 | 17 | Mediator |
| MED20 | Q9H944 | 212 | -1 | Mediator |
| MED21 | Q13503 | 144 | -12 | Mediator |
| MED22 | Q15528 | 200 | -16 | Mediator |
| MED27 | Q6P2C8 | 311 | 9 | Mediator |
| MED28 | Q9H204 | 178 | -4 | Mediator |
| MED29 | Q9NX70 | 200 | -2 | Mediator |
| MED30 | Q96HR3 | 178 | 2 | Mediator |
| MED31 | Q9Y3C7 | 131 | 3 | Mediator |

### REFERENCES

Adelman, K., and Lis, J. T. (2012). Promoter-proximal pausing of RNA polymerase II: emerging roles in metazoans. Nat Rev Genet 13, 720-731.
Adhikari, S., Leaf, M. A., and Muthukumar, M. (2018). Polyelectrolyte complex coacervation by electrostatic dipolar interactions. J Chem Phys 149, 163308.
Andersson, R., Gebhard, C., Miguel-Escalada, I., Hoof, I., Bornholdt, J., Boyd, M., Chen, Y., Zhao, X., Schmidl, C., Suzuki, T., et al. (2014). An atlas of active enhancers across human cell types and tissues. Nature 507, 455-461.
Andrews, J. O., Conway, W., Cho, W.-K., Narayanan, A., Spille, J.-H., Jayanth, N., Inoue, T., Mullen, S., Thaler, J., and Cissé, I. I. (2018). qSR: a quantitative super-resolution analysis tool reveals the cell-cycle dependent organization of RNA Polymerase I in live human cells. Scientific Reports 8, 1-10.
Aumiller, W. M., Pir Cakmak, F., Davis, B. W., and Keating, C. D. (2016). RNA-Based Coacervates as a Model for Membraneless Organelles: Formation, Properties, and Interfacial Liposome Assembly. Langmuir 32, 10042-10053.
Azofeifa, J. G., Allen, M. A., Hendrix, J. R., Read, T., Rubin, J. D., and Dowell, R. D. (2018). Enhancer RNA profiling predicts transcription factor activity. Genome Res 28, 334-344.
Banani, S. F., Lee, H. O., Hyman, A. A., and Rosen, M. K. (2017). Biomolecular condensates: Organizers of cellular biochemistry. Nature Reviews Molecular Cell Biology 18, 285-298.
Banerjee, P. R., Milin, A. N., Moosa, M. M., Onuchic, P. L., and Deniz, A. A. (2017). Reentrant Phase Transition Drives Dynamic Substructure Formation in Ribonucleoprotein Droplets. Angewandte Chemie International Edition 56, 11354-11359.
Bergot, M. O., Diaz-Guerra, M. J., Puzenat, N., Raymondjean, M., and Kahn, A. (1992). Cis-regulation of the L-type pyruvate kinase gene promoter by glucose, insulin and cyclic AMP. Nucleic Acids Res 20, 1871-1877.
Blair, D. G. R. (1985). Activation of mammalian RNA polymerases by polyamines. International Journal of Biochemistry 17, 23-30.
Boeynaems, S., Holehouse, A. S., Weinhardt, V., Kovacs, D., Lindt, J. V., Larabell, C., Bosch, L. V. D., Das, R., Tompa, P. S., Pappu, R. V., et al. (2019). Spontaneous driving forces give rise to protein-RNA condensates with coexisting phases and complex material properties. PNAS 116, 7889-7898.
Boija, A., Klein, I. A., Sabari, B. R., Dall′Agnese, A., Coffey, E. L., Zamudio, A. V., Li, C. H., Shrinivas, K., Manteiga, J. C., Hannett, N. M., et al. (2018). Transcription Factors Activate Genes through the Phase-Separation Capacity of Their Activation Domains. Cell 175, 1842-1855 e16.
Brandman, O., and Meyer, T. (2008). Feedback Loops Shape Cellular Signals in Space and Time. Science 322, 390-395.
Bruhat, A., Jousse, C., Carraro, V., Reimold, A. M., Ferrara, M., and Fafournoux, P. (2000). Amino Acids Control Mammalian Gene Transcription: Activating Transcription Factor 2 Is Essential for the Amino Acid Responsiveness of the CHOP Promoter. Mol Cell Biol 20, 7192-7204.
Cambridge, S. B., Gnad, F., Nguyen, C., Bermejo, J. L., Krüger, M., and Mann, M. (2011). Systems-wide Proteomic Analysis in Mammalian Cells Reveals Conserved, Functional Protein Turnover. J. Proteome Res. 10, 5275-5284.
Carey, M. F., Peterson, C. L., and Smale, S. T. (2009). Transcriptional Regulation in Eukaryotes: Concepts, Strategies, and Techniques, Second Edition.
Catarino, R. R., and Stark, A. (2018). Assessing sufficiency and necessity of enhancer activities for gene expression and the mechanisms of transcription activation. Genes Dev. 32, 202-223.
Chen, H., and Larson, D. R. (2016). What have single-molecule studies taught us about gene expression? Genes Dev. 30, 1796-1810.
Chen, W., Smeekens, J. M., and Wu, R. (2016). Systematic study of the dynamics and half-lives of newly synthesized proteins in human cells. Chem. Sci. 7, 1393-1400.
Chiu, A. C., Suzuki, H. I., Wu, X., Mahat, D. B., Kriz, A. J., and Sharp, P. A. (2018). Transcriptional Pause Sites Delineate Stable Nucleosome-Associated Premature Polyadenylation Suppressed by U1 snRNP. Molecular Cell 69, 648-663.e7.
Cho, W.-K., Jayanth, N., English, B. P., Inoue, T., Andrews, J. O., Conway, W., Grimm, J. B., Spille, J.-H., Lavis, L. D., Lionnet, T., et al. (2016). RNA Polymerase II cluster dynamics predict mRNA output in living cells. ELife 5, e13617.
Cho, W.-K. K., Spille, J.-H. H., Hecht, M., Lee, C., Li, C., Grube, V., Cisse, I. I., Lee, C., Hecht, M., Cho, W.-K. K., et al. (2018). Mediator and RNA polymerase II clusters associate in transcription-dependent condensates. Science 361, 412-415.
Chubb, J. R., Trcek, T., Shenoy, S. M., and Singer, R. H. (2006). Transcriptional Pulsing of a Developmental Gene. Current Biology 16, 1018-1025.
Cisse, I. I., Izeddin, I., Causse, S. Z., Boudarene, L., Senecal, A., Muresan, L., Dugast-Darzacq, C., Hajj, B., Dahan, M., and Darzacq, X. (2013). Real-Time Dynamics of RNA Polymerase II Clustering in Live Human Cells. Science 341, 664-667.
Core, L., and Adelman, K. (2019). Promoter-proximal pausing of RNA polymerase II: a nexus of gene regulation. Genes Dev. 33, 960-982.
Core, L. J., Martins, A. L., Danko, C. G., Waters, C. T., Siepel, A., and Lis, J. T. (2014). Analysis of nascent RNA identifies a unified architecture of initiation regions at mammalian promoters and enhancers. Nat Genet 46, 1311-1320.
Cramer, P. (2019). Organization and regulation of gene transcription. Nature 573, 45-54.
Delaney, K. T., and Fredrickson, G. H. (2017). Theory of polyelectrolyte complexation-Complex coacervates are self-coacervates. J Chem Phys 146, 224902.
Di Tommaso, P., Chatzou, M., Floden, E. W., Barja, P. P., Palumbo, E., and Notredame, C. (2017). Nextflow enables reproducible computational workflows. Nature Biotechnology 35, 316-319.
Dignam, J. D., Lebovitz, R. M., and Roeder, R. G. (1983). Accurate transcription initiation by RNA polymerase II in a soluble extract from isolated mammalian nuclei. Nucleic Acids Res 11, 1475-1489.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Drobot, B., Iglesias-Artola, J. M., Le Vay, K., Mayr, V., Kar, M., Kreysing, M., Mutschler, H., and Tang, T.-Y. D. (2018). Compartmentalised RNA catalysis in membrane-free coacervate protocells. Nat Commun 9, 3643.
Dunlap, J. C. (1999). Molecular Bases for Circadian Clocks. Cell 96, 271-290.
Ebert, B. L., and Bunn, H. F. (1999). Regulation of the erythropoietin gene. Blood 94, 1864-1877.
Elowitz, M. B., and Leibler, S. (2000). A synthetic oscillatory network of transcriptional regulators. Nature 403, 335-338.
Ewels, P. A., Peltzer, A., Fillinger, S., Patel, H., Alneberg, J., Wilm, A., Garcia, M. U., Di Tommaso, P., and Nahnsen, S. (2020). The nf-core framework for community-curated bioinformatics pipelines. Nature Biotechnology 38, 276-278.
Fay, M. M., and Anderson, P. J. (2018). The role of RNA in biological phase separations. J Mol Biol 430, 4685-4701.
Flores, O., Lu, H., and Reinberg, D. (1992). Factors involved in specific transcription by mammalian RNA polymerase II. Identification and characterization of factor IIH. J. Biol. Chem. 267, 2786-2793.
Flory, P. J. (1942). Thermodynamics of High Polymer Solutions. J. Chem. Phys. 10, 51-61.
Frankel, E. A., Bevilacqua, P. C., and Keating, C. D. (2016). Polyamine/Nucleotide Coacervates Provide Strong Compartmentalization of Mg2+, Nucleotides, and RNA. Langmuir 32, 2041-2049.
Fukaya, T., Lim, B., and Levine, M. (2016). Enhancer Control of Transcriptional Bursting. Cell 166, 358-368.
Gardini, A., and Shiekhattar, R. (2015). The many faces of long noncoding RNAs. The FEBS Journal 282, 1647-1657.
Gardner, T. S., Cantor, C. R., and Collins, J. J. (2000). Construction of a genetic toggle switch in Escherichia coli. Nature 403, 339-342.
Gu, B., Swigut, T., Spencley, A., Bauer, M. R., Chung, M., Meyer, T., and Wysocka, J. (2018). Transcription-coupled changes in nuclear mobility of mammalian cis-regulatory elements. Science 359, 1050-1055.
Guo, Y. E., Manteiga, J. C., Henninger, J. E., Sabari, B. R., Dall′Agnese, A., Hannett, N. M., Spille, J.-H., Afeyan, L. K., Zamudio, A. V., Shrinivas, K., et al. (2019). Pol II phosphorylation regulates a switch between transcriptional and splicing condensates. Nature 572, 543-548.
Guyer, J. E., Wheeler, D., and Warren, J. A. (2009). FiPy: Partial Differential Equations with Python. Comput. Sci. Eng. 11, 6-15.
Henriques, T., Scruggs, B. S., Inouye, M. O., Muse, G. W., Williams, L. H., Burkholder, A. B., Lavender, C. A., Fargo, D. C., and Adelman, K. (2018). Widespread transcriptional pausing and elongation control at enhancers. Genes Dev. 32, 26-41.
Hnisz, D., Shrinivas, K., Young, R. A., Chakraborty, A. K., and Sharp, P. A. (2017). A Phase Separation Model for Transcriptional Control. Cell 169, 13-23.
Hohenberg, P. C., and Halperin, B. I. (1977). Theory of dynamic critical phenomena. Rev. Mod. Phys. 49, 435-479.
http://crispr.mit.edu Guide Design Resources.
Jangi, M., and Sharp, P. A. (2014). Building Robust Transcriptomes with Master Splicing Factors. Cell 159, 487-498.
Jin, Y., Eser, U., Struhl, K., and Churchman, L. S. (2017). The Ground State and Evolution of Promoter Region Directionality. Cell 170, 889-898.e10.
Kardar, M. (2007). Statistical physics of fields (Cambridge: Cambridge Univ. Press).
Karolchik, D., Hinrichs, A. S., Furey, T. S., Roskin, K. M., Sugnet, C. W., Haussler, D., and Kent, W. J. (2004). The UCSC Table Browser data retrieval tool. Nucleic Acids Res. 32, D493-496.
Kim, T.-K., Hemberg, M., Gray, J. M., Costa, A. M., Bear, D. M., Wu, J., Harmin, D. A., Laptewicz, M., Barbara-Haley, K., Kuersten, S., et al. (2010). Widespread transcription at neuronal activity-regulated enhancers. Nature 465, 182-187.
Kirk, J. M., Kim, S. O., Inoue, K., Smola, M. J., Lee, D. M., Schertzer, M. D., Wooten, J. S., Baker, A. R., Sprague, D., Collins, D. W., et al. (2018). Functional classification of long non-coding RNAs by k-mer content. Nature Genetics 50, 1474-1482.
Klein, I. A., Boija, A., Afeyan, L. K., Hawken, S. W., Fan, M., Dall′Agnese, A., Oksuz, O., Henninger, J. E., Shrinivas, K., Sabari, B. R., et al. (2020). Partitioning of cancer therapeutics in nuclear condensates. Science 368, 1386-1392.
Lahav, G., Rosenfeld, N., Sigal, A., Geva-Zatorsky, N., Levine, A. J., Elowitz, M. B., and Alon, U. (2004). Dynamics of the p53-Mdm2 feedback loop in individual cells. Nat Genet 36, 147-150.
Lai, F., Orom, U. A., Cesaroni, M., Beringer, M., Taatjes, D. J., Blobel, G. A., and Shiekhattar, R. (2013). Activating RNAs associate with Mediator to enhance chromatin architecture and transcription. Nature 494, 497-501.
Landau, L. D. (1937). ON THE THEORY OF PHASE TRANSITIONS. Zh. Eksp. Teor. Fiz 11, 19.
Langmead, B., Trapnell, C., Pop, M., and Salzberg, S. L. (2009). Ultrafast and memory-efficient alignment of short DNA sequences to the human genome. Genome Biology 10, R25.
Larsson, A. J. M., Johnsson, P., Hagemann-Jensen, M., Hartmanis, L., Faridani, O. R., Reinius, B., Segerstolpe, Å., Rivera, C. M., Ren, B., and Sandberg, R. (2019). Genomic encoding of transcriptional burst kinetics. Nature 565, 251-254.
Leinonen, R., Sugawara, H., and Shumway, M. (2011). The Sequence Read Archive. Nucleic Acids Res 39, D19-D21.
LeRoy, G., Rickards, B., and Flint, S. J. (2008). The Double Bromodomain Proteins Brd2 and Brd3 Couple Histone Acetylation to Transcription. Molecular Cell.
LeRoy, G., Oksuz, O., Descostes, N., Aoi, Y., Ganai, R. A., Kara, H. O., Yu, J.-R., Lee, C.-H., Stafford, J., Shilatifard, A., et al. (2019). LEDGF and HDGF2 relieve the nucleosome-induced barrier to transcription in differentiated cells. Sci Adv 5, eaay3068.
Li, C. H., Coffey, E. L., Dall′Agnese, A., Hannett, N. M., Tang, X., Henninger, J. E., Platt, J. M., Oksuz, O., Zamudio, A. V., Afeyan, L. K., et al. (2020). MeCP2 links heterochromatin condensates and neurodevelopmental disease. Nature.
Li, W., Notani, D., and Rosenfeld, M. G. (2016). Enhancers as non-coding RNA transcription units: recent insights and future perspectives. Nat Rev Genet 17, 207-223.
Liao, Y., Smyth, G. K., and Shi, W. (2014). featureCounts: an efficient general purpose program for assigning sequence reads to genomic features. Bioinformatics 30, 923-930.
Lin, Y., McCarty, J., Rauch, J. N., Delaney, K. T., Kosik, K. S., Fredrickson, G. H., Shea, J.-E., and Han, S. (2019). Narrow equilibrium window for complex coacervation of tau and RNA under cellular conditions. ELife 8, e42571.
Lin, Y., Fichou, Y., Zeng, Z., Hu, N. Y., and Han, S. (2020). Electrostatically Driven Complex Coacervation and Amyloid Aggregation of Tau Are Independent Processes with Overlapping Conditions. ACS Chem Neurosci 11, 615-627.
Maharana, S., Wang, J., Papadopoulos, D. K., Richter, D., Pozniakovsky, A., Poser, I., Bickle, M., Rizk, S., Guillen-Boixet, J., Franzmann, T. M., et al. (2018). RNA buffers the phase separation behavior of prion-like RNA binding proteins. Science 360, 918-921.
Maiuri, P., Knezevich, A., De Marco, A., Mazza, D., Kula, A., McNally, J. G., and Marcello, A. (2011). Fast transcription rates of RNA polymerase II in human cells. EMBO Rep 12, 1280-1285.
Mikhaylichenko, O., Bondarenko, V., Harnett, D., Schor, I. E., Males, M., Viales, R. R., and Furlong, E. E. M. (2018). The degree of enhancer or promoter activity is reflected by the levels and directionality of eRNA transcription. Genes Dev 32, 42-57.
Milin, A. N., and Deniz, A. A. (2018). Reentrant Phase Transitions and Non-Equilibrium Dynamics in Membraneless Organelles. Biochemistry 57, 2470-2477.
Monod, J., and Jacob, F. (1961). General Conclusions: Teleonomic Mechanisms in Cellular Metabolism, Growth, and Differentiation. Cold Spring Harb Symp Quant Biol 26, 389-401.
Moruzzi, G., Barbiroli, B., Moruzzi, M. S., and Tadolini, B. (1975). The effect of spermine on transcription of mammalian chromatin by mammalian deoxyribonucleic acid-dependent ribonucleic acid polymerase. Biochem J 146, 697-703.
Mountain, G. A., and Keating, C. D. (2020). Formation of Multiphase Complex Coacervates and Partitioning of Biomolecules within them. Biomacromolecules 21, 630-640.
Muthukumar, M. (2016). Electrostatic correlations in polyelectrolyte solutions. Polym. Sci. Ser. A 58, 852-863.
Nair, S. J., Yang, L., Meluzzi, D., Oh, S., Yang, F., Friedman, M. J., Wang, S., Suter, T., Alshareedah, I., Gamliel, A., et al. (2019). Phase separation of ligand-activated enhancers licenses cooperative chromosomal enhancer assembly. Nature Structural & Molecular Biology 26, 193-203.
Niewidok, B., Igaev, M., Pereira da Graca, A., Strassner, A., Lenzen, C., Richter, C. P., Piehler, J., Kurre, R., and Brandt, R. (2018). Single-molecule imaging reveals dynamic biphasic partition of RNA-binding proteins in stress granules. J. Cell Biol. 217, 1303-1318.
Nott, T. J., Petsalaki, E., Farber, P., Jervis, D., Fussner, E., Plochowietz, A., Craggs, T. D., Bazett-Jones, D. P., Pawson, T., Forman-Kay, J. D., et al. (2015). Phase Transition of a Disordered Nuage Protein Generates Environmentally Responsive Membraneless Organelles. Molecular Cell 57, 936-947.
Orphanides, G., LeRoy, G., Chang, C.-H., Luse, D. S., and Reinberg, D. (1998). FACT, a Factor that Facilitates Transcript Elongation through Nucleosomes. Cell 92, 105-116.
Overbeek, J. T. G., and Voorn, M. J. (1957). Phase separation in polyelectrolyte solutions. Theory of complex coacervation. Journal of Cellular and Comparative Physiology 49, 7-26.
Pai, D. A., Kaplan, C. D., Kweon, H. K., Murakami, K., Andrews, P. C., and Engelke, D. R. (2014). RNAs nonspecifically inhibit RNA polymerase II by preventing binding to the DNA template. RNA 20, 644-655.
Pefanis, E., Wang, J., Rothschild, G., Lim, J., Kazadi, D., Sun, J., Federation, A., Chao, J., Elliott, O., Liu, Z.-P., et al. (2015). RNA Exosome-Regulated Long Non-Coding RNA Transcription Controls Super-Enhancer Activity. Cell 161, 774-789.
Peran, I., and Mittag, T. (2020). Molecular structure in biomolecular condensates. Current Opinion in Structural Biology 60, 17-26.
Quinlan, A. R., and Hall, I. M. (2010). BEDTools: a flexible suite of utilities for comparing genomic features. Bioinformatics 26, 841-842.
Rahnamoun, H., Lee, J., Sun, Z., Lu, H., Ramsey, K. M., Komives, E. A., and Lauberth, S. M. (2018). RNAs interact with BRD4 to promote enhanced chromatin engagement and transcription activation. Nature Structural & Molecular Biology 25, 687-697.
Raj, A., and van Oudenaarden, A. (2008). Nature, Nurture, or Chance: Stochastic Gene Expression and Its Consequences. Cell 135, 216-226.
Raj, A., Peskin, C. S., Tranchina, D., Vargas, D. Y., and Tyagi, S. (2006). Stochastic mRNA Synthesis in Mammalian Cells. PLOS Biol 4.
Reinberg, D., and Roeder, R. G. (1987). Factors involved in specific transcription by mammalian RNA polymerase II. Transcription factor IIS stimulates elongation of RNA chains. J. Biol. Chem. 262, 3331-3337.
Roden, C., and Gladfelter, A. S. (2020). RNA contributions to the form and function of biomolecular condensates. Nature Reviews Molecular Cell Biology 1-13.
Rodriguez, J., and Larson, D. R. (2020). Transcription in Living Cells: Molecular Mechanisms of Bursting. Annual Review of Biochemistry 89, null.
Roeder, R. G. (2019). 50+ years of eukaryotic transcription: an expanding universe of factors and mechanisms. Nature Structural & Molecular Biology 26, 783-791.
Sabari, B. R., Dall′Agnese, A., Boija, A., Klein, I. A., Coffey, E. L., Shrinivas, K., Abraham, B. J., Hannett, N. M., Zamudio, A. V., Manteiga, J. C., et al. (2018). Coactivator condensation at super-enhancers links phase separation and gene control. Science 361, eaar3958.
Sabari, B. R., Dall′Agnese, A., and Young, R. A. (2020). Biomolecular Condensates in the Nucleus. Trends in Biochemical Sciences 0.
Schaukowitch, K., Joo, J.-Y., Liu, X., Watts, J. K., Martinez, C., and Kim, T.-K. (2014). Enhancer RNA Facilitates NELF Release from Immediate Early Genes. Molecular Cell 56, 29-42.
Scruggs, B. S., Gilchrist, D. A., Nechaev, S., Muse, G. W., Burkholder, A., Fargo, D. C., and Adelman, K. (2015). Bidirectional Transcription Arises from Two Distinct Hubs of Transcription Factor Binding and Active Chromatin. Molecular Cell 58, 1101-1112.
Seila, A. C., Calabrese, J. M., Levine, S. S., Yeo, G. W., Rahl, P. B., Flynn, R. A., Young, R. A., and Sharp, P. A. (2008). Divergent Transcription from Active Promoters. Science 322, 1849-1851.
Sellick, C. A., and Reece, R. J. (2003). Modulation of transcription factor function by an amino acid: activation of Put3p by proline. EMBO J 22, 5147-5153.
Shin, Y., and Brangwynne, C. P. (2017). Liquid phase condensation in cell physiology and disease. Science 357, eaaf4382-eaaf4382.
Shrinivas, K., Sabari, B. R., Coffey, E. L., Klein, I. A., Boija, A., Zamudio, A. V., Schuijers, J., Hannett, N. M., Sharp, P. A., Young, R. A., et al. (2019). Enhancer Features that Drive Formation of Transcriptional Condensates. Molecular Cell 75, 549-561.e7.
Sigova, A. A., Abraham, B. J., Ji, X., Molinie, B., Hannett, N. M., Guo, Y. E., Jangi, M., Giallourakis, C. C., Sharp, P. A., and Young, R. A. (2015). Transcription factor trapping by RNA in gene regulatory elements. Science 350, 978-981.
Sing, C. E. (2017). Development of the modern theory of polymeric complex coacervation. Advances in Colloid and Interface Science 239, 2-16.
Singh, J., and Padgett, R. A. (2009). Rates of in situ transcription and splicing in large human genes. Nat Struct Mol Biol 16, 1128-1133.
Smith, K. N., Miller, S. C., Varani, G., Calabrese, J. M., and Magnuson, T. (2019). Multimodal Long Noncoding RNA Interaction Networks: Control Panels for Cell Fate Specification. Genetics 213, 1093-1110.
Sobell, H. M. (1985). Actinomycin and DNA transcription. Proc Natl Acad Sci USA 82, 5328-5331.
Srivastava, S., and Tirrell, M. V. (2016). Polyelectrolyte complexation. Advances in Chemical Physics 499-544.
Steurer, B., Janssens, R. C., Geverts, B., Geijer, M. E., Wienholz, F., Theil, A. F., Chang, J., Dealy, S., Pothof, J., van Cappellen, W. A., et al. (2018). Live-cell analysis of endogenous GFP-RPB1 uncovers rapid turnover of initiating and promoter-paused RNA Polymerase II. Proc Natl Acad Sci USA 115, E4368-E4376.
Stringer, C., Wang, T., Michaelos, M., and Pachitariu, M. (2020). Cellpose: a generalist algorithm for cellular segmentation. BioRxiv 2020.02.02.931238.
Strom, A. R., and Brangwynne, C. P. (2019). The liquid nucleome-phase transitions in the nucleus at a glance. J Cell Sci 132.
Struhl, K. (2007). Transcriptional noise and the fidelity of initiation by RNA polymerase II. Nat. Struct. Mol. Biol. 14, 103-105.
Suter, D. M., Molina, N., Gatfield, D., Schneider, K., Schibler, U., and Naef, F. (2011). Mammalian Genes Are Transcribed with Widely Different Bursting Kinetics. Science 332, 472-474.
Taylor, N. O., Wei, M.-T., Stone, H. A., and Brangwynne, C. P. (2019). Quantifying Dynamics in Phase-Separated Condensates Using Fluorescence Recovery after Photobleaching. Biophysical Journal 117, 1285-1300.
Tunnacliffe, E., and Chubb, J. R. (2020). What Is a Transcriptional Burst? Trends in Genetics 36, 288-297.
Umbarger, H. E. (1956). Evidence for a Negative-Feedback Mechanism in the Biosynthesis of Isoleucine. Science 123, 848-848.
Weber, C. A., Zwicker, D., Jülicher, F., and Lee, C. F. (2019). Physics of active emulsions. Rep. Prog. Phys. 82, 064601.
Whyte, W. A., Orlando, D. A., Hnisz, D., Abraham, B. J., Lin, C. Y., Kagey, M. H., Rahl, P. B., Lee, T. I., and Young, R. A. (2013). Master Transcription Factors and Mediator Establish Super-Enhancers at Key Cell Identity Genes. Cell 153, 307-319.
Zamudio, A. V., Dall′Agnese, A., Henninger, J. E., Manteiga, J. C., Afeyan, L. K., Hannett, N. M., Coffey, E. L., Li, C. H., Oksuz, O., Sabari, B. R., et al. (2019). Mediator Condensates Localize Signaling Factors to Key Cell Identity Genes. Molecular Cell.
Zhang, P., Shen, K., Alsaifi, N. M., and Wang, Z.-G. (2018). Salt Partitioning in Complex Coacervation of Symmetric Polyelectrolytes. Macromolecules 51, 5586-5593.
Zwicker. D., Seyboldt. R., Weber, C. A., Hyman. A. A., and Jülicher, F. (2017). Growth and division of active droplets provides a model for protocells. Nature Physics/3, 408-413.

1. A method of modulating condensate dependent transcription of a gene in a cell of a subject comprising modulating an amount, effective charge, structure, or behavior of nucleic acid incorporated in the condensate, wherein the modulating of condensate dependent transcription of the gene treats, prevents or reduces the likelihood of a disease or condition in the subject, and wherein the disease or condition is associated with a regulatory RNA (regRNA) variant.

2. The method of claim 1, wherein transcription of the gene is increased by at least 1.1-fold.

3. The method of claim 1, wherein transcription of the gene is reduced by at least 5%.

4. The method of claim 1, wherein the condensate dependent transcription of the gene occurs in the presence of one or more species of regRNA in the condensate.

5. The method of claim 4, wherein the one or more species of regRNA is a tissue specific regRNA.

6. (canceled)

7. The method of claim 4, wherein the nucleic acid comprises one or more species of regRNA.

8. The method of claim 7, wherein the amount of one or more species of regRNA in the condensate is modulated by modulating transcription of the one or more species of regRNA.

9. The method of claim 7, wherein the condensate dependent transcription of the gene is modulated by contacting the one or more species of regRNA with an agent capable of specifically binding to the one or more species of regRNA.

10. The method of claim 9, wherein the agent comprises a nucleic acid having a sequence complementary to a sequence of the one or more species of regRNA.

11. The method of claim 10, wherein the nucleic acid is an antisense oligonucleotide or an antisense RNA.

12. The method of claim 7, wherein the one or more species of regRNA comprises enhancer RNA (eRNA).

13. (canceled)

14. (canceled)

15. (canceled)

16. (canceled)

17. (canceled)

18. (canceled)

19. (canceled)

20. (canceled)

21. The method of claim 1, wherein the regRNA variant is an eRNA variant.

22. (canceled)

23. (canceled)

24. (canceled)

25. (canceled)

26. (canceled)

27. (canceled)

28. (canceled)

29. A method of treating, preventing or reducing the likelihood of a disease or condition in a subject comprising administering to the subject an agent that modulates an amount, effective charge, structure, or behavior of a regulatory RNA (regRNA) in a transcriptional condensate in a cell of the subject, and thereby modulates transcription of a gene in the cell and treats, prevents, or reduces the likelihood of the disease or condition, wherein the disease or condition is associated with an regRNA variant.

30. (canceled)

31. (canceled)

32. (canceled)

33. (canceled)

34. A method of identifying an agent that modulates a condensate, comprising
a. providing a condensate comprising a regulatory RNA (regRNA),
b. contacting the condensate with a test agent, and
c. assessing whether the test agent dissolves or modulates the size of the condensate.

35. (canceled)

36. (canceled)

37. (canceled)

38. (canceled)

39. (canceled)

40. (canceled)

41. A method of identifying an agent that increases condensate formation, comprising
a. providing a composition comprising a regulatory RNA (regRNA) and a condensate component under conditions wherein the concentration of the regRNA or condensate component does not form a condensate,
b. contacting the composition with a test agent, and
c. assessing whether contact with the test agent causes formation of a condensate.

42. (canceled)

43. (canceled)

44. (canceled)

45. (canceled)

46. (canceled)

47. (canceled)

48. A method of identifying an agent that modulates condensate dependent transcription of a gene, comprising
a. providing an in vitro transcription assay with condensate dependent expression of a reporter gene,
b. contacting the in vitro transcription assay with a test agent, and
c. assessing expression of the reporter gene,
wherein condensate dependent expression requires incorporation of a regulatory RNA (regRNA) in the condensate.

49. (canceled)

50. (canceled)

51. (canceled)

52. (canceled)

53. (canceled)

54. (canceled)

55. (canceled)

56. (canceled)

57. (canceled)

58. (canceled)

59. (canceled)


SEQUENCE LISTING (58 sequences)

SEQ ID NO: 1 - DNA, 20 bp, Artificial
  primer
taatacgactcactataggg

SEQ ID NO: 2 - DNA, 20 bp, Artificial
  primer
atttaggtgacactatagaa

SEQ ID NO: 3 - DNA, 20 bp, Artificial
  primer
ggcctagacagcactctcca

SEQ ID NO: 4 - DNA, 20 bp, Artificial
  primer
tggatctctgtgagttcaag

SEQ ID NO: 5 - DNA, 20 bp, Artificial
  primer
aaatcttggagagagtagga

SEQ ID NO: 6 - DNA, 20 bp, Artificial
  primer
gggaaaaagttacagtgacc

SEQ ID NO: 7 - DNA, 20 bp, Artificial
  primer
cttccagaacatctggattt

SEQ ID NO: 8 - DNA, 20 bp, Artificial
  primer
aaaacaaacaaaaaagagtc

SEQ ID NO: 9 - DNA, 20 bp, Artificial
  primer
agcctgccttttggctacca

SEQ ID NO: 10 - DNA, 20 bp, Artificial
  primer
agagtgccaggtcccctgga

SEQ ID NO: 11 - DNA, 21 bp, Artificial
  primer
taggtgagccgtctttccacc

SEQ ID NO: 12 - DNA, 21 bp, Artificial
  primer
cccaattcccttcactgctgc

SEQ ID NO: 13 - DNA, 17 bp, Artificial
  primer
cgggcggtgagaagcgt

SEQ ID NO: 14 - DNA, 22 bp, Artificial
  primer
aatgcatgcacaccctctgatt

SEQ ID NO: 15 - DNA, 20 bp, Artificial
  primer
agaggctcttctggggttgt

SEQ ID NO: 16 - DNA, 20 bp, Artificial
  primer
gcgaacaagtagggccagtt

SEQ ID NO: 17 - DNA, 20 bp, Artificial
  primer
gccctggattgtacctgtcc

SEQ ID NO: 18 - DNA, 21 bp, Artificial
  primer
accttcaaagtgggtaacgct

SEQ ID NO: 19 - DNA, 20 bp, Artificial
  primer
caggttagccctaagcgtgc

SEQ ID NO: 20 - DNA, 20 bp, Artificial
  primer
aggctagggcacatctgttt

SEQ ID NO: 21 - DNA, 21 bp, Artificial
  primer
ccctaagcgtgcctagagtat

SEQ ID NO: 22 - DNA, 19 bp, Artificial
  primer
accaggctagggcacatct

SEQ ID NO: 23 - DNA, 20 bp, Artificial
  primer
gctgctgccctgtctacatt

SEQ ID NO: 24 - DNA, 20 bp, Artificial
  primer
ctggccacccagtactcact

SEQ ID NO: 25 - DNA, 19 bp, Artificial
  primer
gagtactgggtggccaggt

SEQ ID NO: 26 - DNA, 19 bp, Artificial
  primer
ccccctcttaaaccagcag

SEQ ID NO: 27 - DNA, 20 bp, Artificial
  primer
gttggagaaggtggaaccaa

SEQ ID NO: 28 - DNA, 19 bp, Artificial
  primer
cccaattcccttcactgct

SEQ ID NO: 29 - DNA, 20 bp, Artificial
  primer
agagggaacctcctctgagc

SEQ ID NO: 30 - DNA, 20 bp, Artificial
  primer
cagccaagtccctttcactt

SEQ ID NO: 31 - DNA, 20 bp, Artificial
  primer
gatctggcaccacaccttct

SEQ ID NO: 32 - DNA, 18 bp, Artificial
  primer
tggggtgttgaaggtctc

SEQ ID NO: 33 - DNA, 18 bp, Artificial
  primer
agaattcgagctcggtac

SEQ ID NO: 34 - DNA, 20 bp, Artificial
  primer
gcgaattcctagttagctag

SEQ ID NO: 35 - DNA, 20 bp, Artificial
  primer
ttgctcacgaatacgacggt

SEQ ID NO: 36 - DNA, 20 bp, Artificial
  primer
ctgtacatcggtgtggctgt

SEQ ID NO: 37 - DNA, 20 bp, Artificial
  primer
aagaagtgctcgtcctcgtc

SEQ ID NO: 38 - DNA, 20 bp, Artificial
  primer
tacgttaacaaccccgaggc

SEQ ID NO: 39 - DNA, 20 bp, Artificial
  primer
gctcgtagaaggggaggttg

SEQ ID NO: 40 - DNA, 18 bp, Artificial
  primer
caccagggcaagggtctg

SEQ ID NO: 41 - DNA, 20 bp, Artificial
  primer
gccctggattgtacctgtcc

SEQ ID NO: 42 - DNA, 20 bp, Artificial
  primer
accttcaaagtgggtaacgc

SEQ ID NO: 43 - DNA, 20 bp, Artificial
  primer
cctgaagttcatctgcacca

SEQ ID NO: 44 - DNA, 20 bp, Artificial
  primer
gtcttgtaggtgccgtcgtc

SEQ ID NO: 45 - DNA, 2268 bp, Artificial
  primer
agcctgccttttggctaccagccacctcttcgctcggatctttcaccagagactctcaaagacactaaagaggcaggacaggaatgggggttggggagggatccatcgccgtctcctaagcagactcctttgacccggagctgtgcgccctgtaccaaacctttgtagaacttggggtaaacttaaggctatggtggccttgactccgtggacccagaggcaagtttcctcctttagaggactcgcatgcattttgtttctaatttgaaatgagaaccggcttagagcttgaaccagccagttctctggactcctcccagctcttacaattcctctcccggacggttcctagaagacaaaggcaagcttaccaaaattacgtcgcccttgggacacacctagggttccctggtggcatcttttttttttcattataaacaggagtaaatttttgtaagggcagagctggtagctgagggagaggaaccctttggcctagtgaaggtagtttgctgggctttgtatccccgcccccacctcccccgagagagagagagagagagagagagagagagagagagagagagagagagagagagagagagagagagagagagactacgtggttatttcaaaaacttgagtgtggcaaaagtatgtaactgggattagtaagcatttctttccttagtgagattggagtagagggtgggaaaggaccttagaatcctcgaatgttgggcttaggaatggggagacaagagccatcacagaatgcctattgtccttcaatatgttagcgatgggccccgtgctttagattttaggcttgtattttctttgtgtgtgtgtgtgtttgttttgtttctgtttctttaggcagtctggagatcaggctggctttcaactccctgtgatgcccctacctctcctgaggtgtgagtggcagaatgctcagcgggtaaaacacacttgtataagtgcaagagaaccaagttccaaacattgtcctctgacttcctcaggcatgccttagctctaacaaataaaattgaagaaaaggattccaaaccacggtgaaggtgccacatctttgatccccttaaggcagacagaagtagagggatccctgggagttcaaagccaggttggtctgcatactgagttggctagccaggaatacacactgagactctgtctttaaaaaaaaaaaagtgtgaagactgctttctctgtcccagcacttgggagagagggaaagaaacacagcagccagccttgtctacacagtgagttccaggctaaccagatctgacatagtgaggtcctgactcaaaattaaaaattggctacagataatactgtagcccttggttagtccgagtacttaactcaacataccattcttcgtttaagcaaaccacgtgaaagacttttcactgaaggctgcaagtcttaaaatgactttggtgatgcctgcctggactgtctaccctctggagcagacttacaaagaatattttttactaagcgctgcataaaccttgatattttgaacggcctattcattctttgcctaatgacaagaatcacatcagggacatatttgtattagtccagcgaataagcagaaggtagaacagttattcttttgttctatgatttctacttaggggctttagtccatctgtttattgtttaaaaccttcatatctcaccaagtagtggtggccatccctttaaccccagcattctgagagacagaagcaggtgggtctctgtgagttcgaggccagcctggcctacagatctaattccaggatagccggggctacatagagaaactctgtctcaaaataaataagtaaataaataaataagtaaatgatatatttacattatttattatatcacaaattatatacgtgttctatagaatatgttactatatacatattttttttggtttttcgagacagggtttcatatagcattagcttcatatatataatttatatgcaaaaatatctctgaaaatggaatcactggaacccaattctaaaagtattgtttttttttgtttttaaagtttcctcatacctcaaagttgtcagaggagggcttaagagatgggctagaggggctggagagatagctcagcagttaagagctctggttgctcttccagaggaccagggttcaatttccagtacctacatgacagctcataactatctgtaatttcagttccaggggacctggcactct

SEQ ID NO: 46 - DNA, 594 bp, Artificial
  47
aaatcttggagagagtaggacctgagctgtttcctatttctactacttccatagacatccatgggtctcctttggttcttaccttaaactgcagttctagaggctcttctggggttgtaaactgcccttccccaactcaggagctcccattccctccccttcctaaactggccctacttgttcgctctactttatcttgccaggggttgtaacagcctccaatctgtcttggatcaggaagcagtttccatggggtattttaagtgtaagttccccggtgaaatggaaacaaaaagaataaggtattctggggacagtgtaggcccactagggtgtagcttgttttgtaaacacaggacctgagttttggactttgccctggattgtacctgtcccagtgggttaccttagatgacactagcttcataatgcaagaggaggttaagatgctggctagcgttacccactttgaaggtactggaaaaggaagacctgatttacccaaaagagggagatctctgtgcttctaccgctttaattttttttttctttttttaaatatttttgcaggactggtcactgtaactttttccc

SEQ ID NO: 47 - DNA, 477 bp, Artificial
  primer
ggcctagacagcactctccaccacagttcaagtatgcctgcagcccagcagtcctgtctgtattcaataccaaccttgtcttatggattgtgatttctcttttggtgactcactggccaggacaagagacatattctgagtcctttaactgcctagtgactggctttgcaaccaggttagccctaagcgtgcctagagtataatacagtccttaacagcaactttgtctgaagtcccaagtcttcttagaactagcttgaaacagatgtgccctagcctggtcccgagctgtgagcctggtggccctggagatgggacagcagacctgttggctcatctgatccagtttcttgcctcctgggtcttagaaaatcatcagactaacttttgggtttgattcaagggttccctacatagccttagatggcttggaactcactctgtagaccaggctggccttgaactcacagagatcca

SEQ ID NO: 48 - DNA, 855 bp, Artificial
  primer
cttccagaacatctggatttgggaagagacgttgctggtcccagggcggctgggggttggggttgggggagggggatgctaaccagcaaggaagctgttcctggctggggcaggcctgactgagctcatgtcgctgaaactcctcatttctccctatggcttcatagggagacccagcctggatgctaacacgagtgatttccctgctctagtctagtgtcctccgtgagtccatttaactgatcacccagtctgtgaggaggtggctgaactcacagtaagaaagctgtgggggtcaacgcctattgtttgtttgttttgttttagacaaggtctcctgctgaggctggctcaagctggcctggaggactcttgtgtttaaggctggccttaaattctctttaaaagaaaatcatgtgtatatatgtgtgtccatgtaactgcagatgaccacagcagccaagagatttctgttctcctagctgtaaaccacccaatatgggtgctaggaacagaattttaaaagggtcctctgaaagagcaatgtacactcaaatgctgagttctttcccagcccctagccttggacctttgttcttatcacttccaacgcccaagggcaggcattataggtgtggcattccgcatctggcttcccaggataccttttcatgctggtggaccatctctggctggggacgtgtgggcttctctgctgtctttggttctccagacagaactccgagacagatcttgacttggttctaaaatacaggtggtttgtggcaagttaacgaattttagctcaaatttggggtatttaagataccatggtgactcttttttgtttgtttt

SEQ ID NO: 49 - DNA, 490 bp, Artificial
  primer
taggtgagccgtctttccaccaggcccccggctcggggtgcccaccttccccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggaggtaagtgaaagggacttggctgggctggcagaggcagcagtgaagggaattggg

SEQ ID NO: 50 - DNA, 475 bp, Artificial
  primer
cgggcggtgagaagcgtccggctgcttcctcagccgcggcggcctctgcagccgcgtcgtcccctgcggggggcggtggcgaggcgcaggagcttctggagcactgcggcgtgtgtcgcgagcgcctgcggcccgagcgggatcctcggctgctgccctgtctacattcggcctgcagtgcctgcctgggccccgctacacccgccgcagcgaataattcgggggatggcggctcggcgggcgacggcgctagtgagtactgggtggccaggtgcccctccccctcctcgcagcccgtgctcgggactgcgcctgtgcgagagtatgggggcccgggtagggttaagtaggcctgctggtttaagagggggcggggaacgggtcctggcctctgccaatgcccgttaccaggtctggacaccgaggtgcagaatgtgatgggagatgtcaagaaatcagagggtgtgcatgcatt

SEQ ID NO: 51 - DNA, 117 bp, Artificial
  primer
ggttctgccgcaggtggatccggtatgtccaccgccacgacagtcgcccccgcggggatcccggcgaccccgggccctgtgaacccaccccccccggaggtctccaaccccagcaag

SEQ ID NO: 52 - DNA, 1000 bp, Artificial
  primer
atgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacaagcttgcatgcctgcaggtcctcggaggacagtactccgctcggaggacagtactccgctcggaggacagtactccgctcggaggacagtactccgctcggaggacagtactccgactctagaggatccccggtgttcctgaaggggggctataaaagggggtgggggcgcgttcgtcctcactctcttcccctccaagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctccttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggcgaacttcaagatccgccacaacgtcgaggacggcagcgtgcagctcgccgaccactaccagcagaacacccccatc

SEQ ID NO: 53 - protein, 237 aa, Artificial
  primer
SKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYK

SEQ ID NO: 54 - protein, 626 aa, Artificial
  primer
EHHSGSQGPLLTTGDLGKEKTQKRVKEGNGTSNSTLSGPGLDSKPGKRSRTPSNDGKSKDKPPKRKKADTEGKSPSHSSSNRPFTPPTSTGGSKSPGSAGRSQTPPGVATPPIPKITIQIPKGTVMVGKPSSHSQYTSSGSVSSSGSKSHHSHSSSSSSSASTSGKMKSSKSEGSSSSKLSSSMYSSQGSSGSSQSKNSSQSGGKPGSSPITKHGLSSGSSSTKMKPQGKPSSLMNPSLSKPNISPSHSRPPGGSDKLASPMKPVPGTPPSSKAKSPISSGSGGSHMSGTSSSSGMKSSSGLGSSGSLSQKTPPSSNSCTASSSSFSSSGSSMSSSQNQHGSSKGKSPSRNKKPSLTAVIDKLKHGVVTSGPGGEDPLDGQMGVSTNSSSHPMSSKHNMSGGEFQGKREKSDKDKSKVSTSGSSVDSSKKTSESKNVGSTGVAKIIISKHDGGSPSIKAKVTLQKPGESSGEGLRPQMASSKNYGSPLISGSTPKHERGSPSHSKSPAYTPQNLDSESESGSSIAEKSYQNSPSSDDGIRPLPEYSTEKHKKHKKEKKKVKDKDRDRDRDKDRDKKKSHSIKPESWSKSPISSDQSLSMTSNTILSADRPSRLSPDFMIGEEDDDL

SEQ ID NO: 55 - protein, 627 aa, Artificial
  primer
EAASGSQGPLLTTGDLGAEATQAAVAEGNGTSNSTLSGPGLDSAPGAASATPSNDGASADAPPAAAAADTEGASPSASSSNAPFTPPTSTGGSASPGSAGASQTPPGVATPPIPAITIQIPAGTVMVGAPSSASQYTSSGSVSSSGSASAASASSSSSSSASTSGAMASSASEGSSSSALSSSMYSSQGSSGSSQSANSSQSGGAPGSSPITAAGLSSGSSSTAMAPQGAPSSLMNPSLSAPNISPSASAPPGGSDALASPMAPVPGTPPSSAAASPISSGSGGSAMSGTSSSSGMASSSGLGSSGSLSQATPPSSNSCTASSSSFSSSGSSMSSSQNQAGSSAGASPSANAAPSLTAVIDALAAGVVTSGPGGEDPLDGQMGVSTNSSSAPMSSAANMSGGEFQGAAEASDADASAVSTSGSSVDSSAATSESANVGSTGVAAIIISAADGGSPSIAAAVTLQAPGESSGEGLAPQMASSANYGSPLISGSTPAAEAGSPSASASPAYTPQNLDSESESGSSIAEASYQNSPSSDDGIAPLPEYSTEAAAAAAAEAAAVADADADADADADADAAASASIAPESWSASPISSDQSLSMTSNTILSADAPSALSPDFMIGEEDDDLM

SEQ ID NO: 56 - protein, 678 aa, Artificial
  primer
CLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQS

SEQ ID NO: 57 - protein, 360 aa, Artificial
  primer
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 58 - protein, 232 aa, Artificial
  primer
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFPGIWAPPTDVSLGDELHLDGEDVAMAHADALDDFDLDMLGDGDSPGPGFTPHDSAPYGALDMADFEFEQMFTDALGIDEYGG